(19) United States
(12) Patent Application Publication
(10) Pub. No.: US 2026/0259491 A1
(43) Pub. Date: Sep. 3, 2026

(54) RESIST COMPOSITION, RESIST PATTERN FORMATION METHOD, AND SALT

(71) Applicant: TOKYO OHKA KOGYO CO., LTD., Kawasaki-shi (JP)

(72) Inventors: Masatoshi ARAI, Kawasaki-shi (JP); Takatoshi INARI, Kawasaki-shi (JP); Emi UCHIDA, Kawasaki-shi (JP); Makoto SAKATA, Kawasaki-shi (JP)

(21) Appl. No.: 18/877,562

(22) PCT Filed: Jul. 21, 2023
(86) PCT No.: PCT/JP2023/026901

§ 371 Date: Dec. 20, 2024

(30) Foreign Application Priority Data
Jul. 27, 2022 (JP) 2022-119718

(51) Int. Cl. G03F 7/004; G03F 7/038; G03F 7/039

(52) U.S. Cl. CPC G03F 7/0045; G03F 7/038; G03F 7/039

(57) ABSTRACT

A resist composition that generates an acid upon exposure, and whose solubility with respect to a liquid developer changes by an action of the acid. The resist composition includes a base material component whose solubility with respect to a liquid developer changes by an action of an acid, and an acid generating agent component including a compound represented by general formula (b1-1).

### TECHNICAL FIELD

The present invention relates to a resist composition, a method for forming a resist pattern, and a salt.

### BACKGROUND ART

In a lithography technique, for example, a process is performed in which a resist film made of a resist material is formed on a substrate, the resist film is selectively exposed, and a developing treatment is performed to form a resist pattern having a predetermined shape on the resist film. A resist material whose property changes such that an exposed portion of a resist film is dissolved in a liquid developer is referred to as a positive resist material, and a resist material whose property changes such that an exposed portion is not dissolved in a liquid developer is referred to as a negative resist material.

In recent years, in the manufacture of semiconductor elements and liquid crystal display elements, miniaturization of patterns has been more rapidly advanced with the progress of the lithography technique.

A common miniaturization technique is wavelength shortening (energy increase) of a light source for exposure. Specifically, in the related art, ultraviolet rays such as g-line and i-line have been used, but currently mass production of semiconductor elements using KrF excimer lasers and ArF excimer lasers has begun. Studies have been conducted on extreme ultraviolet (EUV), electron beam (EB), X-ray, and the like having a wavelength shorter (an energy higher) than that of the excimer lasers.

The resist material is required to have lithography properties such as a sensitivity to a light source for exposure and resolution capable of reproducing a pattern with a fine dimension.

As a resist material satisfying such requirements, in the related art, a chemically amplified resist composition containing an acid generating agent component that generates an acid upon exposure and a base material component whose solubility with respect to a liquid developer changes by an action of the acid has been used.

A wide variety of acid generating agent components have been proposed so far, and known examples thereof include an onium salt acid generating agent, an oxime sulfonate acid generating agent, a diazomethane acid generating agent, a nitrobenzylsulfonate acid generating agent, an iminosulfonate acid generating agent, and a disulfone acid generating agent.

In order to implement excellent solubility with respect to a liquid developer, good lithography properties, and good resist pattern shape, an acid generating component has been investigated.

### CITATION LIST

### Patent Literature

Patent Literature 1: JP2011-85878A

### SUMMARY OF INVENTION

### Technical Problem

As the lithography technique continues to advance and the miniaturization of resist patterns is further advanced, the resist composition is required to have a high exposure latitude, a high sensitivity to the exposure light source, and stability over time.

However, in the technique in related art, these characteristics have not been sufficiently compatible, and a trade-off between the characteristics has not been resolved.

The present invention has been made in view of the above circumstances, and an object of the present invention is to provide a resist composition capable of forming a resist pattern having excellent stability over time and having both a high exposure latitude and a high sensitivity to a light source for exposure, a method for forming a resist pattern using the resist composition, and a salt useful for the resist composition.

### Solution to Problem

As a result, the present inventors have found that a resist composition capable of forming a resist pattern having excellent stability over time and having both a high exposure latitude and a high sensitivity to a light source for exposure, a method for forming a resist pattern using the resist composition, and a salt useful for the resist composition can be obtained by the following configuration, and have completed the present invention.

That is, the present invention is as follows.

The resist composition according to the embodiment of the present invention is a resist composition that generates an acid upon exposure and whose solubility with respect to a liquid developer changes by an action of the acid, the resist composition including:
a base material component (A) whose solubility with respect to a liquid developer changes by an action of an acid; and
an acid generating agent component (B) that generates an acid upon exposure; in which
the acid generating agent component (B) includes a compound represented by the following general formula (b1-1).

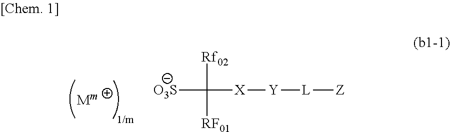

[In the general formula (b1-1), Rf₀₁ and Rf₀₂ each independently represent a hydrogen atom, a fluorine atom, or a fluorinated alkyl group, and at least one of Rf₀₁ and Rf₀₂ contains a fluorine atom. X represents a divalent linking group having a total of 4 or more atoms in a main chain, and the divalent linking group represents an alkylene group which may contain an ester bond or an ether bond, a fluorinated alkylene group which may contain an ester bond or an ether bond, or a combination thereof. Any of ester bonds and ether bonds in X does not form an acetal bond adjacent to each other. Y represents a divalent polycyclic hydrocarbon group that may include a substituent. L represents a divalent linking group containing an ester bond or an oxygen atom, and a plurality of ester bonds and a plurality of oxygen atoms may be each bonded independently via an alkylene group. Z represents a substituent represented by the following formula (bz-1), m represents an integer of 1 or more, and M^{m+} represents an m-valent organic cation.]

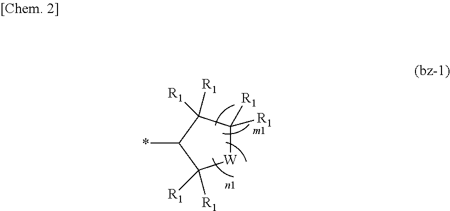

[In the formula (bz-1), R₁'s each independently represent a hydrogen atom, a halogen atom, a linear or branched alkyl group in which a part of carbon atoms may be replaced by a heteroatom, a linear or branched acyl group in which a part of carbon atoms may be replaced by a heteroatom, or a linear or branched alkyloxycarbonyl group in which a part of carbon atoms may be replaced by a heteroatom, W represents an oxygen atom, a sulfur atom, or —NRⁿ—, Rⁿ represents a hydrogen atom, or a linear or branched alkyl group, m1 and n1 each represent an integer of 0 to 4, and m1+n1 represents an integer of 0 to 4, and * represents a bond.]

A method for forming a resist pattern according to another embodiment of the present invention is a method for forming a resist pattern including: a step of forming a resist film on a support using the resist composition according to the embodiment of the present invention; a step of exposing the resist film; and a step of developing the resist film to form a resist pattern.

A salt according to another embodiment of the present invention is represented by the following general formula (b1-1).

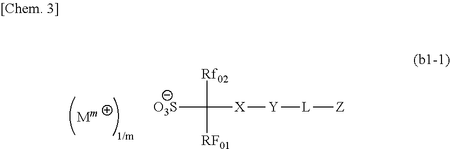

[In the general formula (b1-1), Rf₀₁ and Rf₀₂ each independently represent a hydrogen atom, a fluorine atom, or a fluorinated alkyl group, and at least one of Rf₀₁ and Rf₀₂ contains a fluorine atom. X represents a divalent linking group having a total of 4 or more atoms in a main chain, and the divalent linking group represents an alkylene group which may contain an ester bond or an ether bond, a fluorinated alkylene group which may contain an ester bond or an ether bond, or a combination thereof. Any of ester bonds and ether bonds in X does not form an acetal bond adjacent to each other. Y represents a divalent polycyclic hydrocarbon group that May include a substituent. L represents a divalent linking group containing an ester bond or an oxygen atom, and a plurality of ester bonds and a plurality of oxygen atoms may be each bonded independently via an alkylene group. Z represents a substituent represented by the following formula (bz-1), m represents an integer of 1 or more, and M^{m+} represents an m-valent organic cation.]

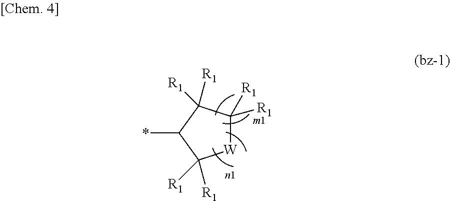

[In the formula (bz-1), R₁'s each independently represent a hydrogen atom, a halogen atom, a linear or branched alkyl group in which a part of carbon atoms may be replaced by a heteroatom, a linear or branched acyl group in which a part of carbon atoms may be replaced by a heteroatom, or a linear or branched alkyloxycarbonyl group in which a part of carbon atoms may be replaced by a heteroatom, W represents an oxygen atom, a sulfur atom, or —NRⁿ—, Rⁿ represents a hydrogen atom, or a linear or branched alkyl group, m1 and n1 each represent an integer of 0 to 4, and m1+n1 represents an integer of 0 to 4, and * represents a bond.]

### Advantageous Effects of Invention

According to the present invention, a resist composition capable of forming a resist pattern having excellent stability over time and having both a high exposure latitude and a high sensitivity to a light source for exposure, a method for forming a resist pattern using the resist composition, and a compound useful for the resist composition can be provided.

### DESCRIPTION OF EMBODIMENTS

Hereinafter, embodiments of the present invention will be described in detail. The present invention is not limited to the embodiments described below.

In the present description and claims, the term “aliphatic” is defined as a relative concept to “aromatic” and means a group, a compound, or the like having no aromaticity.

Unless otherwise specified, the term “alkyl group” includes a linear monovalent saturated hydrocarbon group, a branched monovalent saturated hydrocarbon group, and a cyclic monovalent saturated hydrocarbon group. The same applies to an alkyl group in an alkoxy group.

Unless otherwise specified, the term “alkylene group” includes a linear divalent saturated hydrocarbon group, a branched divalent saturated hydrocarbon group, and a cyclic divalent saturated hydrocarbon group.

The term “halogenated alkyl group” is a group in which a part or all of hydrogen atoms in an alkyl group are replaced by halogen atoms, and examples of the halogen atom include a fluorine atom, a chlorine atom, a bromine atom, and an iodine atom.

The term “fluorinated alkyl group” or “fluorinated alkylene group” refers to a group in which a part or all of hydrogen atoms in an alkyl group or alkylene group are replaced by fluorine atoms.

The term “structural unit” refers to a monomer unit that constitutes a polymer compound (a resin, a polymer, or a copolymer).

The expression “may include a substituent” includes both a case where a hydrogen atom (—H) is replaced by a monovalent group and a case where a methylene group (—CH₂—) is replaced by a divalent group.

The term “exposure” is intended to include a general concept of irradiation with radiation rays.

The expression “structural unit derived from an acrylic ester” means a structural unit formed by cleavage of an ethylenic double bond of an acrylic ester.

The “acrylic ester” is a compound in which a hydrogen atom at a terminal carboxy group of acrylic acid (CH₂═CH—COOH) is replaced by an organic group.

In the acrylic acid ester, a hydrogen atom bonded to a carbon atom at an α-position may be replaced by a substituent. A substituent (R^{α0}) for replacing the hydrogen atom bonded to the carbon atom at the α-position is an atom other than a hydrogen atom or a group, and examples thereof include an alkyl group having 1 to 5 carbon atoms and a halogenated alkyl group having 1 to 5 carbon atoms. The acrylic acid ester also includes an itaconic acid diester in which the substituent (R^{α0}) is replaced by a substituent including an ester bond, and an α-hydroxy acrylic ester in which the substituent (R^{α0}) is replaced by a hydroxyalkyl group or a group modified with a hydroxy group thereof. Unless otherwise specified, the carbon atom at the α-position of the acrylic acid ester refers to a carbon atom to which a carbonyl group of acrylic acid is bonded.

Hereinafter, an acrylic acid ester in which the hydrogen atom bonded to the carbon atom at the α-position is replaced by a substituent may be referred to as an α-replaced acrylic acid ester. In addition, the acrylic acid ester and the α-replaced acrylic acid ester may be collectively referred to as “(α-replaced) acrylic acid ester”.

An alkyl group as the substituent at the α-position is preferably a linear or branched alkyl group, and specific examples thereof include an alkyl group having 1 to 5 carbon atoms (a methyl group, an ethyl group, a propyl group, an isopropyl group, an n-butyl group, an isobutyl group, a tert-butyl group, a pentyl group, an isopentyl group, and a neopentyl group).

Specific examples of a halogenated alkyl group as the substituent at the α-position include a group in which a part or all of hydrogen atoms in the “alkyl group as the substituent at the α-position” are replaced by halogen atoms. Examples of the halogen atom include a fluorine atom, a chlorine atom, a bromine atom, and an iodine atom. A fluorine atom is particularly preferred.

Specific examples of a hydroxyalkyl group as the substituent at the α-position include a group in which a part or all of hydrogen atoms in the “alkyl group as the substituent at the α-position” are replaced by hydroxy groups. The number of hydroxy groups in the hydroxyalkyl group is preferably 1 to 5, and most preferably 1.

In the present disclosure, a numerical range indicated using the symbol “-” or word “to” means a range that includes numerical values written before and after the symbol “-” or word “to” as the lower limit value and the upper limit value, respectively.

Further, in the present disclosure, when a plurality of substances corresponding to each component are present in a composition, an amount of each component in the composition means a total amount of a plurality of corresponding substances present in the composition, unless otherwise specified.

A chemical structural formula in the present disclosure may be described as a simplified structural formula in which hydrogen atoms are omitted.

In the present description and claims, a chiral carbon may exist and hence enantiomers or diastereomers may exist depending on a structure of a chemical formula. In that case, one chemical formula represents all the isomers. The isomers may be used alone or as a mixture.

In the present disclosure, “mass %” and “weight %” have the same meaning, and “parts by mass” and “parts by weight” have the same meaning.

### [Resist Composition]

A resist composition according to the embodiment of the present invention is a resist composition that generates an acid upon exposure and whose solubility with respect to a liquid developer changes by an action of the acid, the resist composition including:
a base material component (A) whose solubility with respect to a liquid developer changes by an action of an acid; and
an acid generating agent component (B) that generates an acid upon exposure; in which
the acid generating agent component (B) includes a compound represented by the following general formula (b1-1).

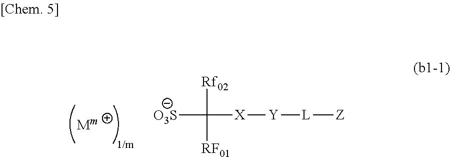

[In the general formula (b1-1), Rf₀₁ and Rf₀₂ each independently represent a hydrogen atom, a fluorine atom, or a fluorinated alkyl group, and at least one of Rf₀₁ and Rf₀₂ contains a fluorine atom. X represents a divalent linking group having a total of 4 or more atoms in a main chain, and the divalent linking group represents an alkylene group which may contain an ester bond or an ether bond, a fluorinated alkylene group which may contain an ester bond or an ether bond, or a combination thereof. Any of ester bonds and ether bonds in X does not form an acetal bond adjacent to each other. Y represents a divalent polycyclic hydrocarbon group that may include a substituent. L represents a divalent linking group containing an ester bond or an oxygen atom, and a plurality of ester bonds and a plurality of oxygen atoms may be each bonded independently via an alkylene group. Z represents a substituent represented by the following formula (bz-1), m represents an integer of 1 or more, and M^{m+} represents an m-valent organic cation.]

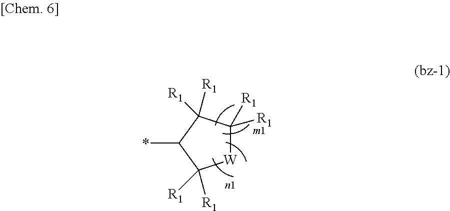

[In the formula (bz-1), R₁'s each independently represent a hydrogen atom, a halogen atom, a linear or branched alkyl group in which a part of carbon atoms may be replaced by a heteroatom, a linear or branched acyl group in which a part of carbon atoms may be replaced by a heteroatom, or a linear or branched alkyloxycarbonyl group in which a part of carbon atoms may be replaced by a heteroatom, W represents an oxygen atom, a sulfur atom, or —NRⁿ—, Rⁿ represents a hydrogen atom, or a linear or branched alkyl group, m1 and n1 each represent an integer of 0 to 4, and m1+n1 represents an integer of 0 to 4, and * represents a bond.]

When a resist film is formed using the resist composition according to the embodiment of the present invention and the resist film is selectively exposed, an acid is generated in an exposed portion of the resist film, and a solubility of the component (A) with respect to a liquid developer changes by an action of the acid, whereas the solubility of the component (A) with respect to the liquid developer does not change in an unexposed portion of the resist film, resulting in a difference in solubility with respect to the liquid developer between the exposed portion and the unexposed portion of the resist film. Therefore, when the resist film is developed, the exposed portion of the resist film is dissolved and removed to form a positive resist pattern in the case where the resist composition is a positive resist composition, and the unexposed portion of the resist film is dissolved and removed to form a negative resist pattern in the case where the resist composition is a negative resist composition.

In the present description, a resist composition that forms a positive resist pattern by dissolving and removing an exposed portion of a resist film is referred to as a positive resist composition, and a resist composition that forms a negative resist pattern by dissolving and removing an unexposed portion of a resist film is referred to as a negative resist composition.

The resist composition according to the embodiment of the present invention may be a positive resist composition or a negative resist composition.

In addition, the resist composition according to the embodiment of the present invention may be for an alkali developing process in which an alkali liquid developer is used in a developing treatment at the time of resist pattern formation, or may be for a solvent developing process in which a liquid developer (an organic liquid developer) containing an organic solvent is used in the developing treatment.

The resist composition according to the embodiment of the present invention is a resist composition that generates an acid upon exposure and whose solubility with respect to a liquid developer changes by an action of the acid. The resist composition contains the base material component (A) whose solubility with respect to the liquid developer changes by the action of the acid, and the acid generating agent component (B) that generates the acid upon exposure.

Examples of the component (A) whose solubility with respect to a liquid developer changes by the action of the acid include known components.

The component (A) may generate an acid upon exposure, and in this case, the component (A) is a “base material component that generates an acid upon exposure and whose solubility with respect to a liquid developer changes by an action of the acid”. When the component (A) is a base material component that generates an acid upon exposure and whose solubility with respect to a liquid developer changes by an action of the acid, a component (A1) to be described later is preferably a macromolecular compound that generates an acid upon exposure and whose solubility with respect to a liquid developer changes by an action of the acid. As such a polymer compound, a copolymer including a structural unit that generates an acid upon exposure can be used.

In the resist composition according to the embodiment of the present invention, the solubility of the base material component (A) with respect to the liquid developer changes by the action of the acid, and therefore in an alkaline developing process, a solubility of the resist film with respect to an alkaline liquid developer increases during development.

The resist composition according to the embodiment of the present invention contains the acid generating agent component (B) that generates an acid upon exposure. The acid generating agent component (B) includes a compound (b1-1) represented by the following general formula (b1-1).

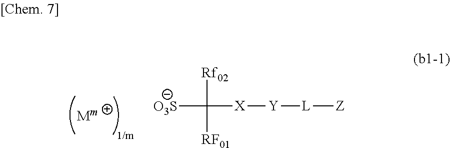

[In the general formula (b1-1), Rf₀₁ and Rf₀₂ each independently represent a hydrogen atom, a fluorine atom, or a fluorinated alkyl group, and at least one of Rf₀₁ and Rf₀₂ contains a fluorine atom. X represents a divalent linking group having a total of 4 or more atoms in a main chain, and the divalent linking group represents an alkylene group which may contain an ester bond or an ether bond, a fluorinated alkylene group which may contain an ester bond or an ether bond, or a combination thereof. Any of ester bonds and ether bonds in X does not form an acetal bond adjacent to each other. Y represents a divalent polycyclic hydrocarbon group that may include a substituent. L represents a divalent linking group containing an ester bond or an oxygen atom, and a plurality of ester bonds and a plurality of oxygen atoms may be each bonded independently via an alkylene group. Z represents a substituent represented by the following formula (bz-1), m represents an integer of 1 or more, and M^{m+} represents an m-valent organic cation.]

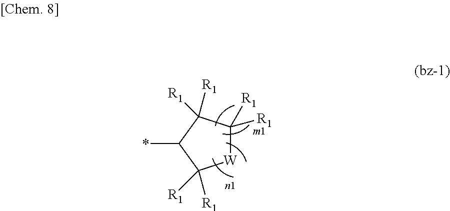

[In the formula (bz-1), R₁'s each independently represent a hydrogen atom, a halogen atom, a linear or branched alkyl group in which a part of carbon atoms may be replaced by a heteroatom, a linear or branched acyl group in which a part of carbon atoms may be replaced by a heteroatom, or a linear or branched alkyloxycarbonyl group in which a part of carbon atoms may be replaced by a heteroatom, W represents an oxygen atom, a sulfur atom, or —NRⁿ—, Rⁿ represents a hydrogen atom, or a linear or branched alkyl group, m1 and n1 each represent an integer of 0 to 4, and m1+n1 represents an integer of 0 to 4, and * represents a bond.]

The compound (b1-1) has an excellent sensitivity when the total number of atoms in the main chain of X is 4 or more. This is presumably because the protecting group and the sulfonic acid moiety can be brought into sufficient proximity to each other to improve reactivity. Since X is a divalent linking group having a specific structure, the acid generating agent component (B) has excellent stability over time. This is presumably because the chain structure represented by X does not contain an acetal bond or a carbonate bond, which prevents decomposition over time. Further, in the compound (b1-1), a polycyclic hydrocarbon group represented by Y and a monocyclic group represented by Z in one molecule are bonded via an ester bond represented by L or a divalent linking group containing an oxygen atom, and therefore, acid diffusion can be appropriately prevented. As a result, the resist composition according to the embodiment of the present invention has excellent stability over time and can form a resist pattern having both a high exposure latitude and a high sensitivity to the exposure light source.

Here, the acetal bond in the present application refers to a bond represented by —O—C—O— in R1-O—C—O—R2 (R1, R2: alkyl group, fluoroalkyl group, or carbonyl group).

### <<Component (A)>>

In the resist composition according to the embodiment of the present invention, the component (A) is a base material component whose solubility with respect to the liquid developer changes by the action of the acid.

The component (A) may be one whose solubility with respect to the liquid developer increases by the action of the acid, or one whose solubility with respect to the liquid developer decreases by the action of the acid. By using the component (A), a polarity of the base material component changes before and after the exposure, so that a good development contrast can be obtained not only in the alkaline developing process but also in the solvent developing process.

As the component (A), a component containing the polymer compound (A1) (hereinafter, also referred to as “component (A1)”) having a structural unit (a1) containing an acid-decomposable group whose polarity increases by the action of the acid is more preferred.

As the component (A1), a polymer compound having the structural unit (a1) and a structural unit (a2) containing a lactone-containing cyclic group, a cyclic group containing —SO₂—, or a carbonate-containing cyclic group is preferably used.

When the alkaline developing process is applied, the base material component containing the component (A1) is poorly soluble with respect to the alkaline liquid developer before exposure, and when an acid is generated from the component (B) upon exposure, the polarity thereof increases by the action of the acid, thereby increasing the solubility with respect to the alkaline liquid developer. Therefore, in forming a resist pattern, when a resist film obtained by coating a support with the resist composition is selectively exposed, an exposed portion of the resist film changes from poorly soluble to soluble with respect to the alkaline liquid developer, whereas an unexposed portion of the resist film remains poorly soluble with respect to alkaline and does not change. Therefore, a positive resist pattern can be formed by alkaline development.

On the other hand, when the solvent developing process is applied, the base material component (A) containing the component (A1) has a high solubility with respect to the organic liquid developer before exposure, and when an acid is generated from the component (B) upon exposure, the polarity thereof increases by the action of the acid, and the solubility with respect to the organic liquid developer decreases. Therefore, in forming a resist pattern, when a resist film obtained by coating a support with the resist composition is selectively exposed, an exposed portion of the resist film changes from soluble to poorly soluble with respect to the organic liquid developer, whereas an unexposed portion of the resist film remains soluble and does not change. Therefore, by developing with the organic liquid developer, a contrast can be created between the exposed portion and the unexposed portion, and a negative resist pattern can be formed.

In the resist composition according to the embodiment of the present invention, the component (A) may be used alone or in combination of two or more kinds thereof.

<Structural Unit (a1)>

The structural unit (a1) is a structural unit containing an acid-decomposable group whose polarity increases by the action of the acid.

The “acid-decomposable group” is a group having an acid-decomposable property in which at least a part of bonds in the structure of the acid-decomposable group can be cleaved by the action of the acid.

Examples of the acid-decomposable group whose polarity increases by the action of the acid include a group that is decomposed by the action of the acid to generate a polar group.

Examples of the polar group include a carboxy group, a hydroxy group, an amino group, and a sulfo group (—SO₃H). Among them, a polar group containing —OH in the structure (hereinafter, may be referred to as an “OH-containing polar group”) is preferred, a carboxy group or a hydroxy group is more preferred, and a carboxy group is particularly preferred.

Specific examples of the acid-decomposable group include a group in which the polar group is protected with an acid-dissociable group (for example, a group in which a hydrogen atom of an OH-containing polar group is protected with an acid-dissociable group).

Here, the term “acid-dissociable group” refers to both (i) a group having an acid dissociable property in which a bond between the acid-dissociable group and an atom adjacent to the acid-dissociable group can be cleaved by an action of an acid, and (ii) a group in which a bond between the acid-dissociable group and an atom adjacent to the acid dissociable-group can be cleaved by further causing a decarboxylation reaction after a part of bonds is cleaved by the action of the acid.

The acid-dissociable group constituting the acid-decomposable group needs to be a group having a polarity lower than that of a polar group generated by dissociation of the acid-dissociable group. Accordingly, when the acid-dissociable group is dissociated by the action of the acid, a polar group having a polarity higher than that of the acid-dissociable group is generated, thereby increasing the polarity. As a result, the polarity of the entire component (A1) increases. When the polarity increases, the solubility with respect to the liquid developer relatively changes, the solubility increases when the liquid developer is an alkaline liquid developer, and the solubility decreases when the liquid developer is an organic liquid developer.

The structural unit (a1) preferably contains an acid-decomposable group having an alicyclic hydrocarbon group, and more preferably contains an acid-decomposable group having a monocyclic alicyclic hydrocarbon group.

Since the acid-decomposable group (the acid-dissociable group) in the structural unit (a1) has an appropriate bulkiness, the acid diffusion control and the solubility with respect to the liquid developer can be appropriately adjusted, and the roughness in forming a resist pattern can be reduced.

Examples of the acid-dissociable group in the structural unit (a1) include those proposed as acid-dissociable groups for base resins for chemically amplified resists in the related art.

Specific examples of those proposed as acid-dissociable groups for base resins for chemically amplified resist compositions include an “acetal acid-dissociable group”, a “tertiary alkyl ester acid-dissociable group” and a “tertiary alkyloxycarbonyl acid-dissociable group”.

Tertiary alkyl ester acid-dissociable group:

Among the polar groups, examples of the acid-dissociable group that protects a carboxy group include an acid-dissociable group represented by the following general formula (a1-r-2).

Among the acid-dissociable group represented by the following formula (a1-r-2), those constituted by an alkyl group may be referred to as a “tertiary alkyl ester acid-dissociable group” hereinafter for convenience.

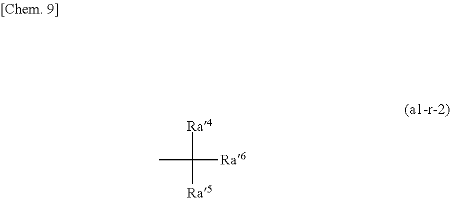

[In the formula, Ra′⁴ to Ra′⁶ each represent a hydrocarbon group, and Ra′⁵ and Ra′⁶ may be bonded to each other to form a ring.]

Examples of the hydrocarbon group represented by Ra′⁴ include a linear or branched alkyl group, a chain or cyclic alkenyl group, or a cyclic hydrocarbon group.

The linear alkyl group preferably has 1 to 5 carbon atoms, more preferably has 1 to 4 carbon atoms, and still more preferably has 1 or 2 carbon atoms. Specific examples thereof include a methyl group, an ethyl group, an n-propyl group, an n-butyl group, and an n-pentyl group. Among them, a methyl group, an ethyl group, or an n-butyl group is preferred, and a methyl group or an ethyl group is more preferred.

The branched alkyl group preferably has 3 to 10 carbon atoms, and more preferably has 3 to 5 carbon atoms. Specific examples thereof include an isopropyl group, an isobutyl group, a tert-butyl group, an isopentyl group, a neopentyl group, a 1,1-diethylpropyl group, and a 2,2-dimethylbutyl group. An isopropyl group is preferred.

When Ra′⁴ represents a cyclic hydrocarbon group (an aliphatic hydrocarbon group which is a monocyclic group, an aliphatic hydrocarbon group which is a polycyclic group, or an aromatic hydrocarbon group), the hydrocarbon group may be an aliphatic hydrocarbon group or an aromatic hydrocarbon group, and may be a polycyclic group or a monocyclic group.

The aliphatic hydrocarbon group which is a monocyclic group is preferably a group in which one hydrogen atom is removed from a monocycloalkane. The monocycloalkane preferably has 3 to 6 carbon atoms, and specific examples thereof include cyclopentane and cyclohexane.

The aliphatic hydrocarbon group which is a polycyclic group is preferably a group in which one hydrogen atom is removed from a polycycloalkane. The polycycloalkane is preferably a group having 7 to 12 carbon atoms, and specific examples thereof include adamantane, norbomane, isobomnane, tricyclodecane, and tetracyclododecane.

When the cyclic hydrocarbon group for Ra′⁴ is an aromatic hydrocarbon group, the aromatic hydrocarbon group is a hydrocarbon group having at least one aromatic ring.

The aromatic ring is not particularly limited as long as it is of a cyclic conjugated system having 4n+2π electrons, and may be monocyclic or polycyclic. The number of carbon atoms of the aromatic ring is preferably 5 to 30, more preferably 5 to 20, still more preferably 6 to 15, and particularly preferably 6 to 12.

Specific examples of the aromatic ring include an aromatic hydrocarbon ring such as benzene, naphthalene, anthracene, and phenanthrene; and an aromatic heterocyclic ring in which a part of carbon atoms constituting the aromatic hydrocarbon ring is replaced by a heteroatom. Examples of the heteroatom in the aromatic heterocyclic ring include an oxygen atom, a sulfur atom, and a nitrogen atom. Specific examples of the aromatic heterocyclic ring include a pyridine ring and a thiophene ring.

Specific examples of the aromatic hydrocarbon group in Ra′⁴ include a group in which one hydrogen atom is removed from the aromatic hydrocarbon ring or aromatic heterocyclic ring (an aryl group or a heteroaryl group); a group in which one hydrogen atom is removed from an aromatic compound containing two or more aromatic rings (for example, biphenyl or fluorene); and a group in which one hydrogen atom in the aromatic hydrocarbon ring or aromatic heterocyclic ring is replaced by an alkylene group (for example, an arylalkyl group such as a benzyl group, a phenethyl group, a 1-naphthyl methyl group, a 2-naphthyl methyl group, a 1-naphthyl ethyl group, or a 2-naphthyl ethyl group). The number of carbon atoms of the alkylene group bonded to the aromatic hydrocarbon ring or aromatic heterocyclic ring is preferably 1 to 4, more preferably 1 to 2, and particularly preferably 1.

The cyclic hydrocarbon group in Ra′⁴ may have a substituent. Examples of the substituent include —R^{P1}, —R^{P2}—O—R^{P1}, —R^{P2}—CO—R^{P1}—R^{P2}—CO—OR^{P1}, —R^{P2}—O—CO—R^{P1}, —R^{P2}—OH, —R^{P2}—CN, and —R^{P2}—COOH) (hereinafter, these substituents are collectively referred to as “Ra^{x5}”).

Here, R^{P1} is a monovalent chained saturated hydrocarbon group having 1 to 10 carbon atoms, a monovalent aliphatic cyclic saturated hydrocarbon group having 3 to 20 carbon atoms, or a monovalent aromatic hydrocarbon group having 6 to 30 carbon atoms. R^{P2} is a single bond, a divalent chain saturated hydrocarbon group having 1 to 10 carbon atoms, a divalent aliphatic cyclic saturated hydrocarbon group having 3 to 20 carbon atoms, or a divalent aromatic hydrocarbon group having 6 to 30 carbon atoms. A part or all of hydrogen atoms of the chain saturated hydrocarbon group, the aliphatic cyclic saturated hydrocarbon group, and the aromatic hydrocarbon group for R^{P1} and R^{P2} may be replaced by fluorine atoms. The aliphatic cyclic hydrocarbon group may have one or more substituents of one kind mentioned above, or may have one or more substituents of each of two or more kinds mentioned above.

Examples of the monovalent chain saturated hydrocarbon group having 1 to 10 carbon atoms include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms include monocyclic aliphatic saturated hydrocarbon groups such as a cyclopropyl group, a cyclobutyl group, a cyclopentyl group, a cyclohexyl group, a cycloheptyl group, a cyclooctyl group, a cyclodecyl group, and a cyclododecyl group; and polycyclic aliphatic saturated hydrocarbon groups such as a bicyclo[2.2.2]octanyl group, a tricyclo[5.2.1.02,6]decanyl group, tricyclo[3.3.1.13,7]decanyl group, a tetracyclo[6.2.1.13,6.02,7]dodecanyl group, and an adamantyl group.

Examples of the monovalent aromatic hydrocarbon group having 6 to 30 carbon atoms include a group in which one hydrogen atom is removed from an aromatic hydrocarbon ring such as benzene, biphenyl, fluorene, naphthalene, anthracene, and phenanthrene.

The chain or cyclic alkenyl group in Ra′⁴ is preferably an alkenyl group having 2 to 10 carbon atoms.

Examples of the hydrocarbon group for Ra′⁵ and Ra′⁶ include groups same as those described above for Ra′⁴.

When Ra′⁵ and Ra′⁶ are bonded to each other to form a ring, suitable examples of the acid-dissociable group represented by the above formula (a1-r-2) include a group represented by the following formula (a1-r2-1), a group represented by the following formula (a1-r2-2), and a group represented by the following formula (a1-r2-3).

On the other hand, when Ra′⁴ to Ra′⁶ are not bonded to each other and are independent a hydrocarbon group, suitable examples of the acid-dissociable group represented by the above formula (a1-r-2) include a group represented by the following formula (a1-r2-4).

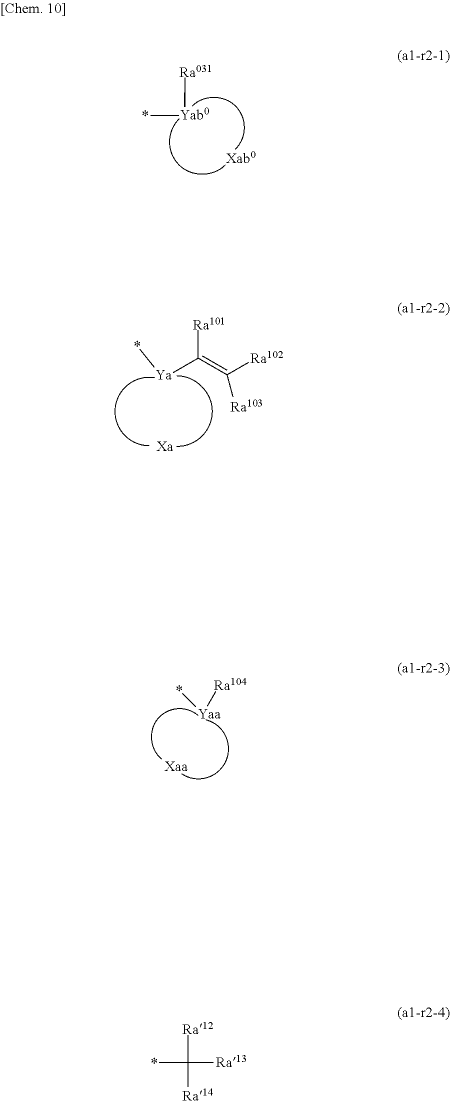

[In the formula (a1-r2-1), Ra⁰³¹ represents an alkyl group, and Yab⁰ represents a carbon atom. Xab⁰ represents a group that forms an alicyclic hydrocarbon group together with Yab⁰, and a part or all of hydrogen atoms of the alicyclic hydrocarbon group may be replaced. In the formula (a1-12-2), Ya represents a carbon atom. Xa is a group that forms a cyclic hydrocarbon group together with Ya. A part or all of hydrogen atoms of the cyclic hydrocarbon group may be replaced. Ra¹⁰¹ to Ra¹⁰³ each independently represent a hydrogen atom, a monovalent chain saturated hydrocarbon group having 1 to 10 carbon atoms, or a monovalent aliphatic cyclic saturated hydrocarbon group having 3 to 20 carbon atoms. A part or all of hydrogen atoms of the chain saturated hydrocarbon group and the aliphatic cyclic saturated hydrocarbon group may be replaced. Two or more of Ra¹⁰¹ to Ra¹⁰³ may be bonded to each other to form a cyclic structure. In the formula (a1-r2-3), Yaa represents a carbon atom. Xaa is a group that forms an aliphatic cyclic group together with Yaa. Ra¹⁰⁴ represents an aromatic hydrocarbon group that may include a substituent. In the formula (a1-r2-4), Ra′¹² and Ra′¹³ each independently represent a monovalent chain saturated hydrocarbon group having 1 to 10 carbon atoms. A part or all of hydrogen atoms in the chain saturated hydrocarbon group may be replaced. Ra′¹⁴ is a hydrocarbon group that may include a substituent. * indicates a bond.]

In the formula (a1-r2-1), Ra⁰³¹ is preferably a chain alkyl group, and is preferably a linear or branched alkyl group having 1 to 12 carbon atoms which may be partially replaced by a halogen atom or a heteroatom-containing group.

The linear alkyl group in Ra⁰³¹ has 1 to 12 carbon atoms, preferably has 1 to 10 carbon atoms, and particularly preferably has 1 to 5 carbon atoms.

Examples of the branched alkyl group in Ra⁰³¹ include groups same as those described above for Ra′⁴.

The alkyl group in Ra⁰³¹ may be partially replaced by a halogen atom or a heteroatom-containing group. For example, a part of hydrogen atoms constituting the alkyl group may be replaced by a halogen atom or a heteroatom-containing group. A part of carbon atoms (such as methylene groups) constituting the alkyl group may be replaced by a heteroatom-containing group.

Examples of the heteroatom used here include an oxygen atom, a sulfur atom, and a nitrogen atom. Examples of the heteroatom-containing group include (—O—), —C(═O)—O—O. C(═O)—, —C(═O)—, —O—C(═O)—O—, —C(═O)—NH—, —NH—, —S—, —S(═O)₂—, and —S(═O)₂—O—.

In the formula (a1-r2-1), Xab⁰ (a group which forms an alicyclic hydrocarbon group together with Yab⁰) is preferably a group described as the aliphatic hydrocarbon group (an alicyclic hydrocarbon group) which is a monocyclic group or polycyclic group for Ra′⁴ in the formula (a1-r-2). Among them, an alicyclic hydrocarbon group is preferred, a monocyclic alicyclic hydrocarbon group is more preferred, and a group in which two or more hydrogen atoms are removed from a monocycloalkane is still more preferred. The monocycloalkane preferably has 3 to 8 carbon atoms, and specific examples thereof include cyclopentane, cyclohexane, cycloheptane, and cyclooctane.

In the formula (a1-r2-2), examples of the cyclic hydrocarbon group formed by Xa together with Ya include a group in which one or more hydrogen atoms are further removed from the cyclic monovalent hydrocarbon group (the aliphatic hydrocarbon group) in Ra′⁴ in the above formula (a1-r-2).

The cyclic hydrocarbon group formed by Xa together with Ya may have a substituent. Examples of the substituent include those same as the substituent that the cyclic hydrocarbon group in Ra′⁴ may have.

Examples of the monovalent chain saturated hydrocarbon group having 1 to 10 carbon atoms in Ra¹⁰¹ to Ra¹⁰³ in the formula (a1-r2-2) include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms in Ra¹⁰¹ to Ra¹⁰³ include monocyclic aliphatic saturated hydrocarbon groups such as a cyclopropyl group, a cyclobutyl group, a cyclopentyl group, a cyclohexyl group, a cycloheptyl group, a cyclooctyl group, a cyclodecyl group, and a cyclododecyl group; and polycyclic aliphatic saturated hydrocarbon groups such as a bicyclo[2.2.2]octanyl group, a tricyclo[5.2.1.02,6]decanyl group, a tricyclo[3.3.1.13,7]decanyl group, a tetracyclo[6.2.1.13,6.02,7]dodecanyl group, and an adamantyl group.

Among them, from the viewpoint of ease of synthesis, Ra¹⁰¹ to Ra¹⁰³ are preferably a hydrogen atom or a monovalent chain saturated hydrocarbon group having 1 to 10 carbon atoms, more preferably a hydrogen atom, a methyl group, or an ethyl group, and particularly preferably a hydrogen atom.

Examples of the substituent that the chain saturated hydrocarbon group or the aliphatic cyclic saturated hydrocarbon group represented by any one of Ra¹⁰¹ to Ra¹⁰³ may include groups same as those represented by Ra^{x5} above.

Examples of the group containing a carbon-carbon double bond generated by two or more of Ra¹⁰¹ to Ra¹⁰³ bonding to each other to form a cyclic structure include a cyclopentenyl group, a cyclohexenyl group, a methylcyclopentenyl group, a methylcyclohexenyl group, a cyclopentylideneethenyl group, and a cyclohexylideneethenyl group. Among them, from the viewpoint of ease of synthesis, a cyclopentenyl group, a cyclohexenyl group, and a cyclopentylideneethenyl group are preferred.

In the formula (a1-r2-3), the aliphatic cyclic group formed by Xaa together with Yaa is preferably a group described as the aliphatic hydrocarbon group which is a monocyclic group or a polycyclic group for Ra′⁴ in the formula (a1-r-2).

Examples of the aromatic hydrocarbon group in Ra¹⁰⁴ in the formula (a1-12-3) include a group in which one or more hydrogen atoms are removed from an aromatic hydrocarbon ring having 5 to 30 carbon atoms. Among them, Ra¹⁰⁴ is preferably a group in which one or more hydrogen atoms are removed from an aromatic hydrocarbon ring having 6 to 15 carbon atoms, more preferably a group in which one or more hydrogen atoms are removed from benzene, naphthalene, anthracene, or phenanthrene, still more preferably a group in which one or more hydrogen atoms are removed from benzene, naphthalene, or anthracene, particularly preferably a group in which one or more hydrogen atoms are removed from benzene or naphthalene, and most preferably a group in which one or more hydrogen atoms are removed from benzene.

Examples of the substituent that Ra¹⁰⁴ in the formula (a1-12-3) may have include a methyl group, an ethyl group, a propyl group, a hydroxy group, a carboxy group, a halogen atom, an alkoxy group (such as a methoxy group, an ethoxy group, a propoxy group, and a butoxy group), and an alkyloxycarbonyl group.

In the formula (a1-r2-4), Ra′¹² and Ra′13 each independently represent a monovalent chain saturated hydrocarbon group having 1 to 10 carbon atoms. Examples of the monovalent chained saturated hydrocarbon group having 1 to 10 carbon atoms in Ra′¹² and Ra′¹³ include those same as the monovalent chained saturated hydrocarbon group having 1 to 10 carbon atoms in Ra¹⁰¹ to Ra¹⁰³. A part or all of hydrogen atoms of the chain saturated hydrocarbon group may be replaced.

Among them, Ra′¹² and Ra′¹³ are more preferably an alkyl group having 1 to 5 carbon atoms, still more preferably a methyl group or an ethyl group, and particularly preferably a methyl group.

When the chain saturated hydrocarbon groups represented by Ra′¹² and Ra′¹³ are replaced, examples of the substituent include groups same as those described above for Ra^{x5}.

In the formula (a1-r2-4), Ra′¹⁴ represents a hydrocarbon group that may include a substituent. Examples of the hydrocarbon group in Ra′¹⁴ include a linear or branched alkyl group and a cyclic hydrocarbon group.

The linear alkyl group in Ra′¹⁴ preferably has 1 to 5 carbon atoms, more preferably has 1 to 4 carbon atoms, and still more preferably has 1 or 2 carbon atoms. Specific examples thereof include a methyl group, an ethyl group, an n-propyl group, an n-butyl group, and an n-pentyl group. Among them, a methyl group, an ethyl group, or an n-butyl group is preferred, and a methyl group or an ethyl group is more preferred.

The branched alkyl group in Ra′¹⁴ preferably has 3 to 10 carbon atoms, and more preferably has 3 to 5 carbon atoms. Specific examples thereof include an isopropyl group, an isobutyl group, a tert-butyl group, an isopentyl group, a neopentyl group, a 1,1-diethylpropyl group, and a 2,2-dimethylbutyl group. An isopropyl group is preferred.

When Ra′¹⁴ is a cyclic hydrocarbon group, the hydrocarbon group may be an aliphatic hydrocarbon group or an aromatic hydrocarbon group, and may be a polycyclic group or a monocyclic group.

The aliphatic hydrocarbon group which is a monocyclic group is preferably a group in which one hydrogen atom is removed from a monocycloalkane. The monocycloalkane preferably has 3 to 6 carbon atoms, and specific examples thereof include cyclopentane and cyclohexane.

The aliphatic hydrocarbon group which is a polycyclic group is preferably a group in which one hydrogen atom is removed from a polycycloalkane. The polycycloalkane is preferably a group having 7 to 12 carbon atoms, and specific examples thereof include adamantane, norbornane, isobornane, tricyclodecane, and tetracyclododecane.

Examples of the aromatic hydrocarbon group in Ra′¹⁴ include those same as the aromatic hydrocarbon group in Ra¹⁰⁴. Among them, Ra′¹⁴ is preferably a group in which one or more hydrogen atoms are removed from an aromatic hydrocarbon ring having 6 to 15 carbon atoms, more preferably a group in which one or more hydrogen atoms are removed from benzene, naphthalene, anthracene, or phenanthrene, still more preferably a group in which one or more hydrogen atoms are removed from benzene, naphthalene, or anthracene, particularly preferably a group in which one or more hydrogen atoms are removed from naphthalene or anthracene, and most preferably a group in which one or more hydrogen atoms are removed from naphthalene.

Examples of the substituent that Ra′¹⁴ may have include those same as the substituents that Ra¹⁰⁴ may have.

When Ra′¹⁴ in the formula (a1-r2-4) is a naphthyl group, a position at which Ra³⁴ bonds to a tertiary carbon atom in the formula (a1-r2-4) may be either 1-position or 2-position of the naphthyl group.

When Ra′¹⁴ in the formula (a1-r2-4) is an anthryl group, a position at which Ra′¹⁴ bonds to a tertiary carbon atom in the formula (a1-12-4) may be any of 1-position, 2-position, and 9-position of the anthryl group.

Specific examples of the group represented by the formula (a1-r2-1) are shown below.

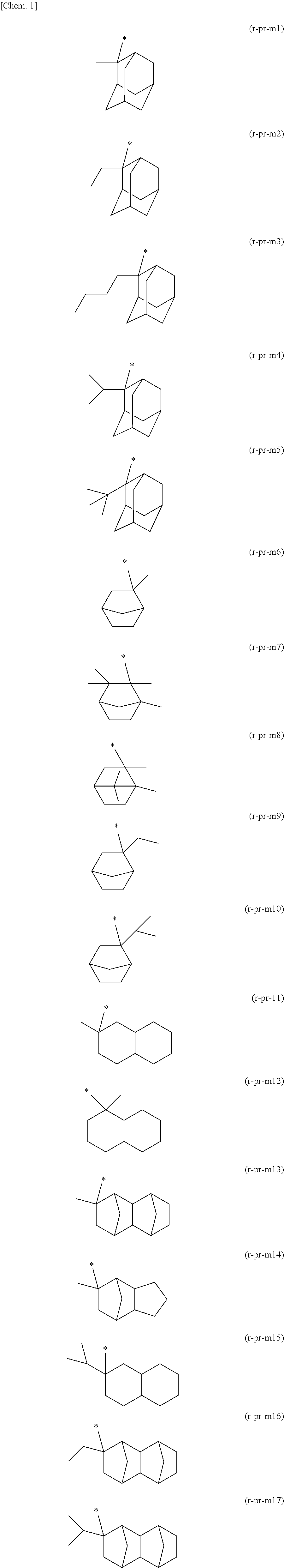

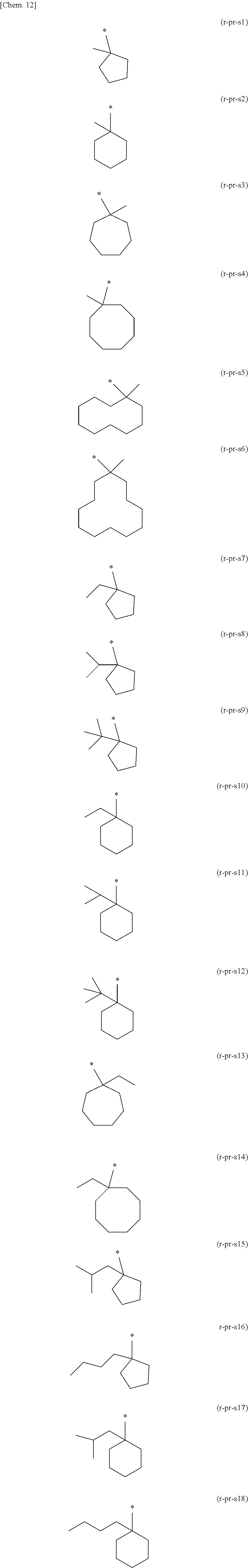

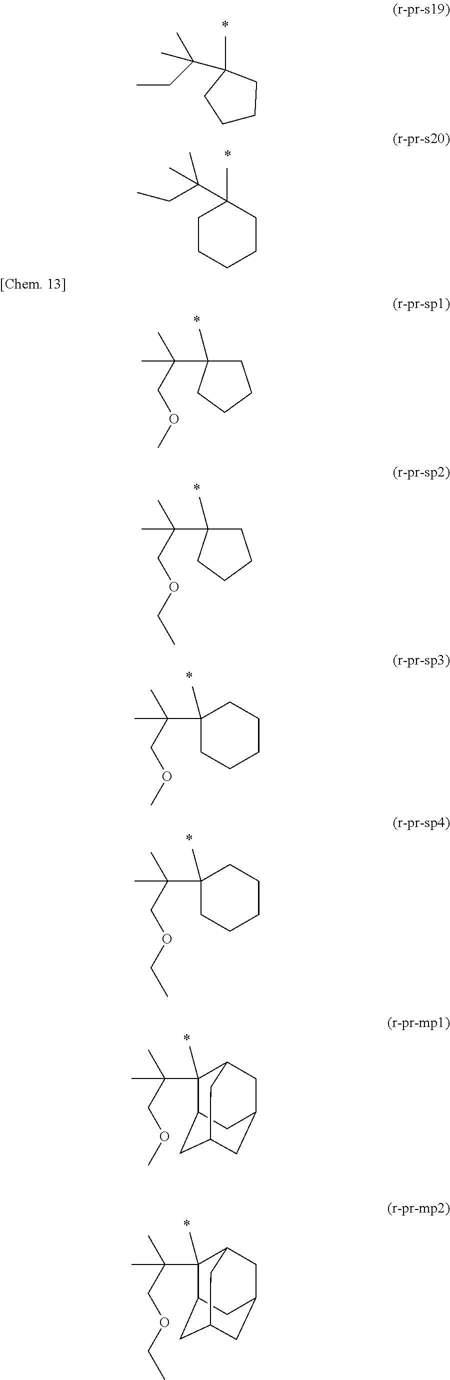

Specific examples of the group represented by the formula (a1-r2-2) are shown below.

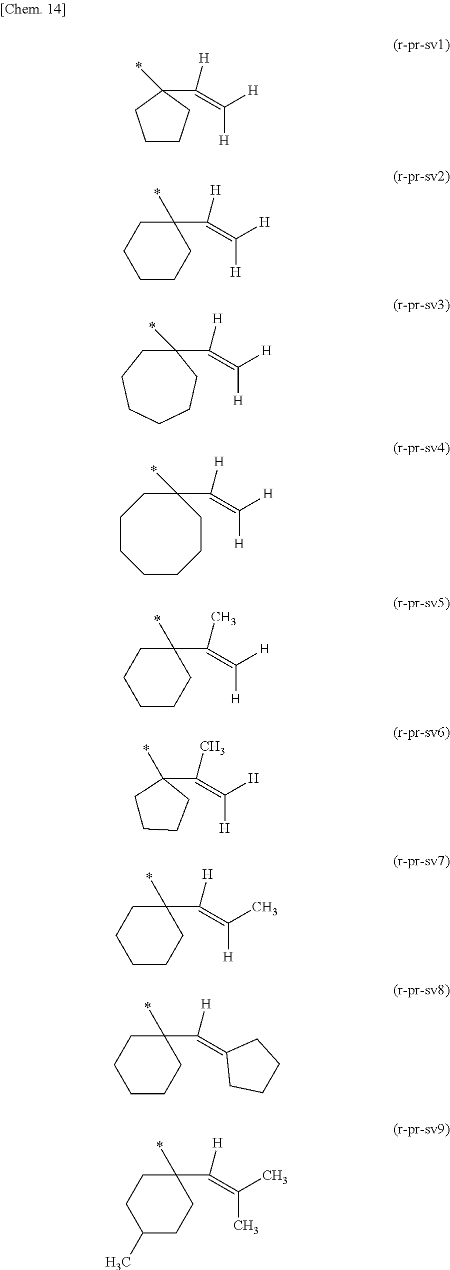

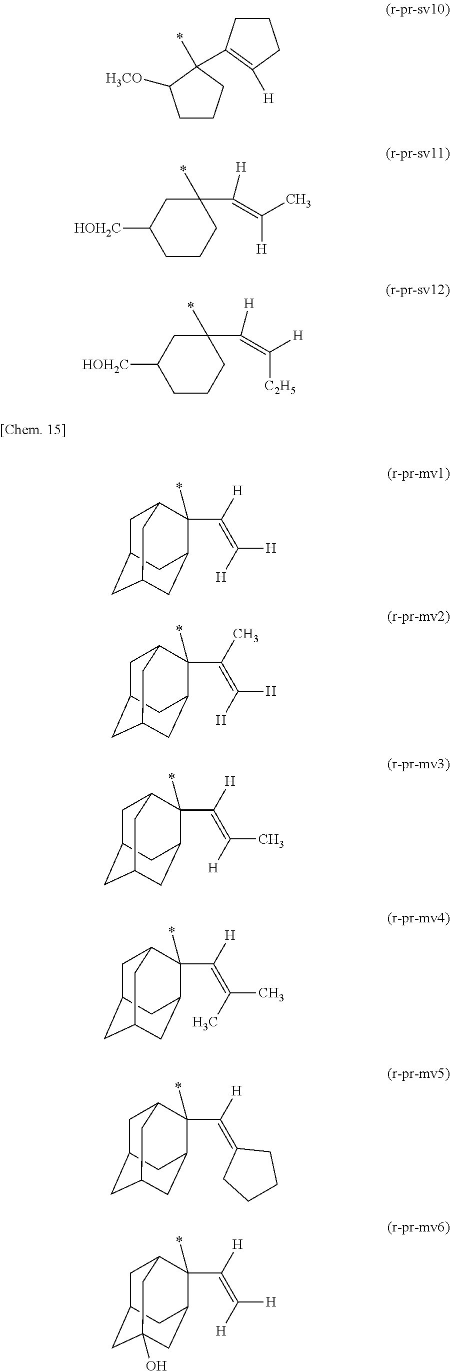

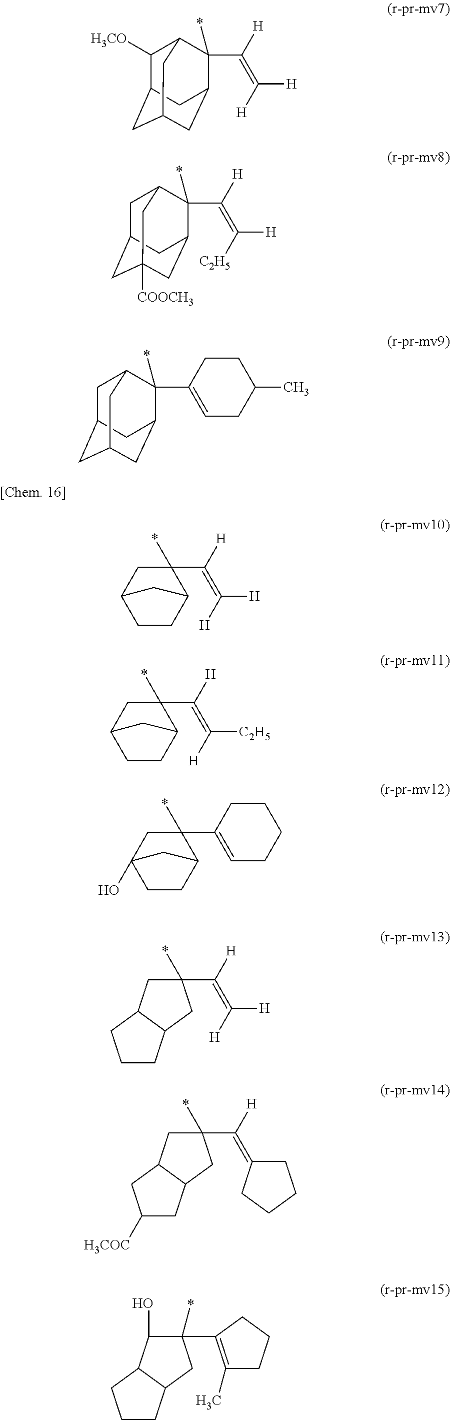

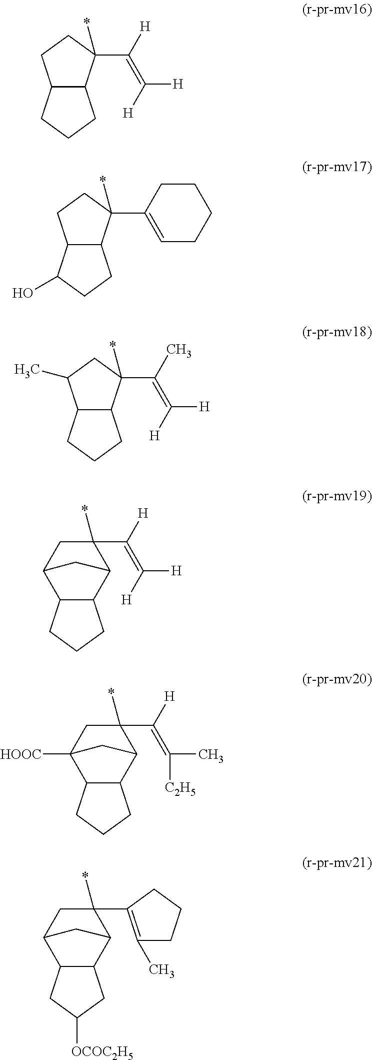

Specific examples of the group represented by the formula (a1-12-3) are shown below.

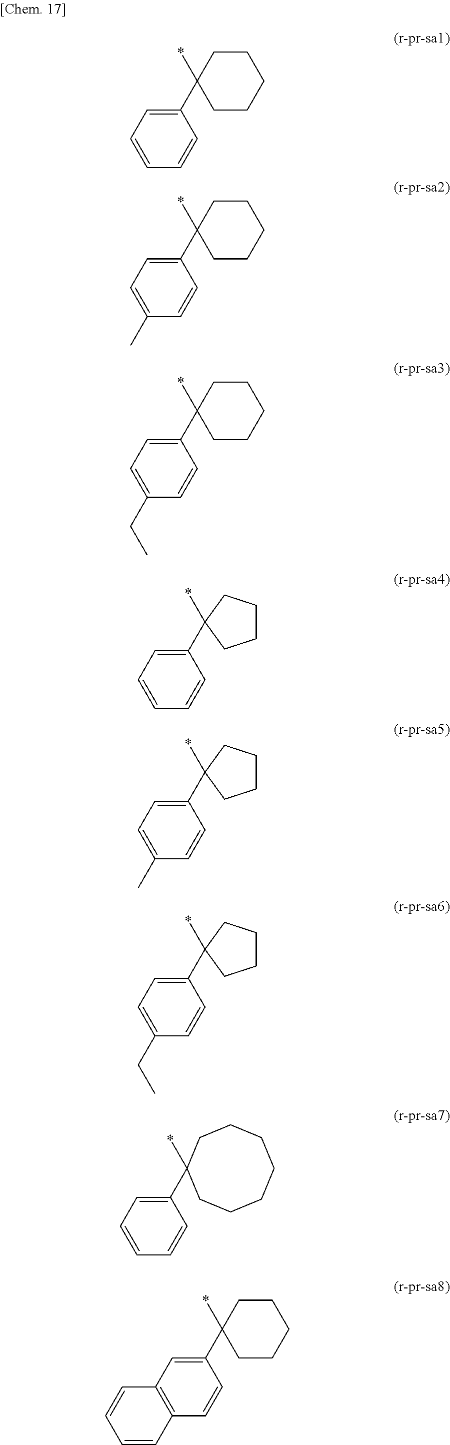

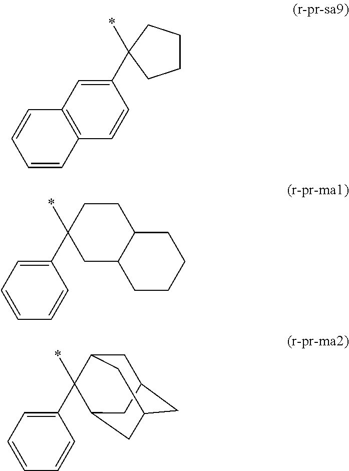

Specific examples of the group represented by the formula (a1-r2-4) are shown below.

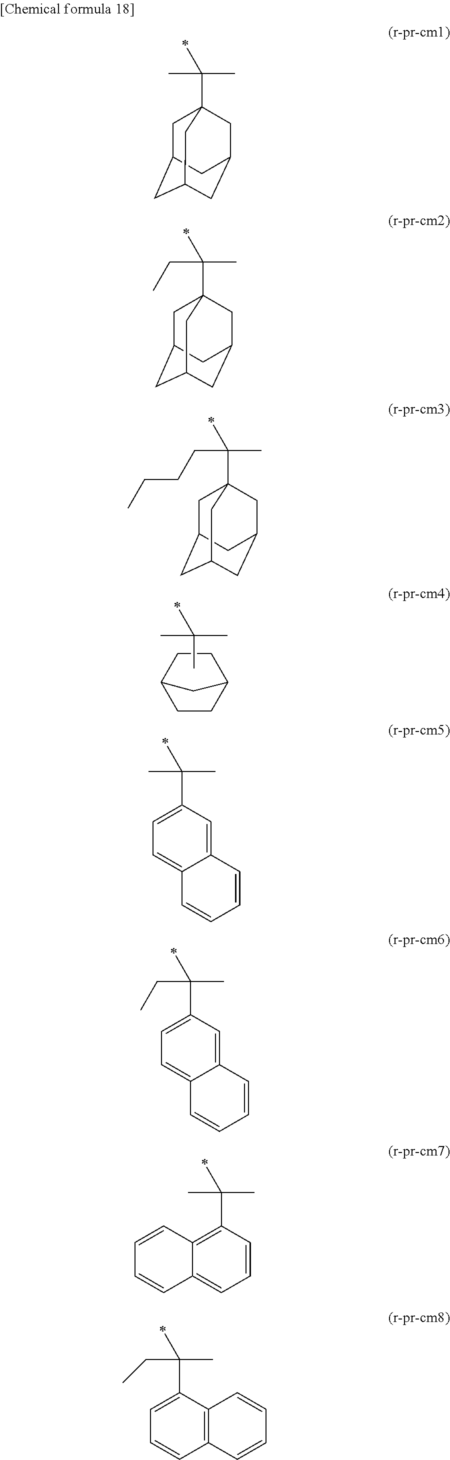

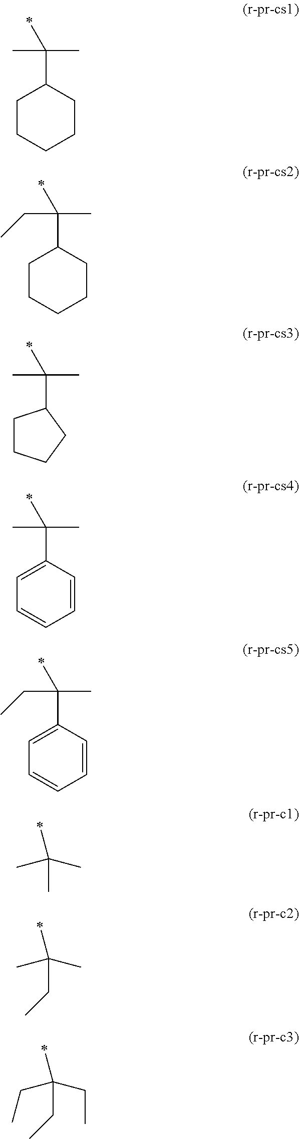

As the acid-dissociable group, a group represented by the general formula (a1-r2-1) is preferred among the group represented by any one of the general formulae (a1-r2-1) to (a1-r2-4).

That is, the base material component (A) according to the embodiment of the present invention preferably contains the polymer compound (A1) including a structural unit containing an acid-dissociable group represented by the formula (a1-r2-1).

Specific examples of the structural unit (a1) include a structural unit represented by the following general formula (a1-1).

(Structural Unit (a1) Represented by General Formula (a1-1))

In the resist composition according to the embodiment of the present invention, the base material component (A) preferably includes the polymer compound (A1) including the structural unit (a1) represented by the following general formula (a1-1).

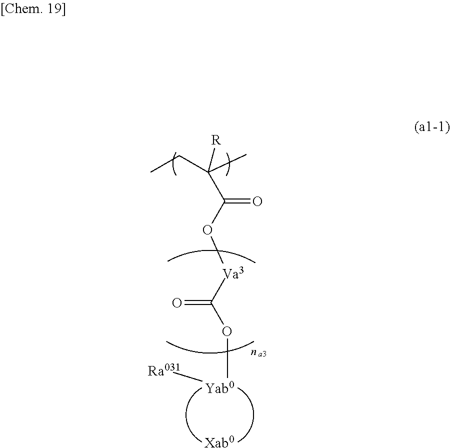

(In the general formula (a1-1), R represents a hydrogen atom, an alkyl group having 1 to 5 carbon atoms, or a halogenated alkyl group having 1 to 5 carbon atoms, Va³ represents a divalent linking group, na₃ represents an integer of 0 to 2, Ra⁰³¹ represents an alkyl group, and Yab⁰ represents a carbon atom, Xab⁰ represents a group which forms an alicyclic hydrocarbon group together with Yab⁰, and a part or all of hydrogen atoms of the alicyclic hydrocarbon group may be replaced.)

In the general formula (a1-1), R represents a hydrogen atom, an alkyl group having 1 to 5 carbon atoms, or a halogenated alkyl group having 1 to 5 carbon atoms.

The alkyl group having 1 to 5 carbon atoms and being represented by R is preferably a linear or branched alkyl group having 1 to 5 carbon atoms, and specific examples thereof include a methyl group, an ethyl group, a propyl group, an iso-propyl group, an n-butyl group, an iso-butyl group, a tert-butyl group, a pentyl group, an iso-pentyl group, and a neopentyl group. The halogenated alkyl group having 1 to 5 carbon atoms is a group in which a part or all of hydrogen atoms of the alkyl group having 1 to 5 carbon atoms are replaced by halogen atoms. The halogen atom is particularly preferably a fluorine atom.

R is preferably a hydrogen atom, an alkyl group having 1 to 5 carbon atoms, or a fluorinated alkyl group having 1 to 5 carbon atoms, and from the viewpoint of industrial availability, a hydrogen atom or a methyl group is most preferred.

In the general formula (a1-1), Va³ represents a divalent linking group.

Examples of the divalent linking group include a divalent hydrocarbon group that may include an ether bond. The divalent hydrocarbon group in Va³ may be an aliphatic hydrocarbon group or an aromatic hydrocarbon group.

The aliphatic hydrocarbon group as the divalent hydrocarbon group in Va³ may be saturated or unsaturated, and is generally preferably saturated.

More specific examples of the aliphatic hydrocarbon group include a linear or branched aliphatic hydrocarbon group, and an aliphatic hydrocarbon group containing a ring in the structure thereof.

The linear aliphatic hydrocarbon group preferably has 1 to 10 carbon atoms, more preferably has 1 to 6 carbon atoms, still more preferably has 1 to 4 carbon atoms, and most preferably has 1 to 3 carbon atoms.

The linear aliphatic hydrocarbon group is preferably a linear alkylene group, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group preferably has 2 to 10 carbon atoms, more preferably has 3 to 6 carbon atoms, still more preferably has 3 or 4 carbon atoms, and most preferably has 3 carbon atoms.

The branched aliphatic hydrocarbon group is preferably a branched alkylene group, and specific examples thereof include alkyl alkylene groups such as alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkyl ethylene groups such as —CH(CH₃)CH₂, —CH(CH₃)CH(CH₃)—, —C(CH₃)₂CH₂—, —CH(CH₂CH₃)CH₂—, and —C(CH₂CH₃)₂—CH₂—; alkyl trimethylene groups such as —CH(CH₃)CH₂CH₂— and —CH₂CH(CH₃)CH₂; and alkyl tetramethylene groups such as —CH(CH₃)CH₂CH₂CH₂— and —CH₂CH(CH₃)CH₂CH₂. The alkyl group in the alkylalkylene group is preferably a linear alkyl group having 1 to 5 carbon atoms.

Examples of the aliphatic hydrocarbon group containing a ring in the structure thereof include an alicyclic hydrocarbon group (a group in which two hydrogen atoms are removed from an aliphatic hydrocarbon ring), a group in which an alicyclic hydrocarbon group is bonded to the end of a linear or branched aliphatic hydrocarbon group, and a group in which an alicyclic hydrocarbon group is interposed in the middle of a linear or branched aliphatic hydrocarbon group. Examples of the linear or branched aliphatic hydrocarbon group include those same as the linear aliphatic hydrocarbon group or the branched aliphatic hydrocarbon group.

The alicyclic hydrocarbon group preferably has 3 to 20 carbon atoms, and more preferably has 3 to 12 carbon atoms.

The alicyclic hydrocarbon group may be polycyclic or monocyclic. The monocyclic alicyclic hydrocarbon group is preferably a group in which two hydrogen atoms are removed from a monocycloalkane. The monocycloalkane preferably has 3 to 6 carbon atoms, and specific examples thereof include cyclopentane and cyclohexane. The polycyclic alicyclic hydrocarbon group is preferably a group in which two hydrogen atoms are removed from a polycycloalkane. The polycycloalkane is preferably a group having 7 to 12 carbon atoms. Specific examples thereof include adamantane, norbornane, isobornane, tricyclodecane, and tetracyclododecane.

The aromatic hydrocarbon group as the divalent hydrocarbon group in Va³ is a hydrocarbon group having an aromatic ring.

The aromatic hydrocarbon group preferably has 3 to 30 carbon atoms, more preferably has 5 to 30 carbon atoms, still more preferably has 5 to 20 carbon atoms, particularly preferably has 6 to 15 carbon atoms, and most preferably has 6 to 12 carbon atoms. The number of the carbon atoms does not include the number of the carbon atoms in the substituent.

Specific examples of the aromatic ring contained in the aromatic hydrocarbon group include an aromatic hydrocarbon ring such as benzene, biphenyl, fluorene, naphthalene, anthracene, and phenanthrene; and an aromatic heterocyclic ring in which a part of carbon atoms constituting the aromatic hydrocarbon ring is replaced by a heteroatom. Examples of the heteroatom in the aromatic heterocyclic ring include an oxygen atom, a sulfur atom, and a nitrogen atom.

Specific examples of the aromatic hydrocarbon group include a group in which two hydrogen atoms are removed from the aromatic hydrocarbon ring (an arylene group); a group in which one hydrogen atom of a group in which one hydrogen atom is removed from the aromatic hydrocarbon ring (an aryl group) is replaced by an alkylene group (for example, a group in which one hydrogen atom is further removed from the aryl group in an arylalkyl group such as a benzyl group, a phenethyl group, a 1-naphthylmethyl group, a 2-naphthylmethyl group, a 1-naphthylethyl group, or a 2-naphthylethyl group). The number of carbon atoms of the alkylene group (an alkyl chain in the arylalkyl group) is preferably 1 to 4, more preferably 1 to 2, and particularly preferably 1.

In the general formula (a1-1), na₃ is an integer of 0 to 2, preferably 0 or 1, and more preferably 0.

In the formulae (a1-12-1) and (a1-1), Ra⁰³¹ is as defined above.

In the formula (a1-12-1) and the general formula (a1-1), among them, Ra⁰³¹ is preferably a chain alkyl group, and is preferably a monovalent chain alkyl group having 1 to 3 carbon atoms. Specifically, a methyl group, an ethyl group, a propyl group, or an iso-propyl group is more preferred.

In the formula (a1-r2-1) and the general formula (a1-1), Yab⁰ represents a carbon atom.

In the formula (a1-r2-1) and the general formula (a1-1), Xab⁰ represents a group which forms an alicyclic hydrocarbon group together with Yab⁰, and a part of all of hydrogen atoms in the alicyclic hydrocarbon group may be replaced.

Specific examples of the structural unit (a1) are shown below.

In the following formulae, R^{α} represents a hydrogen atom, a methyl group, or a trifluoromethyl group.

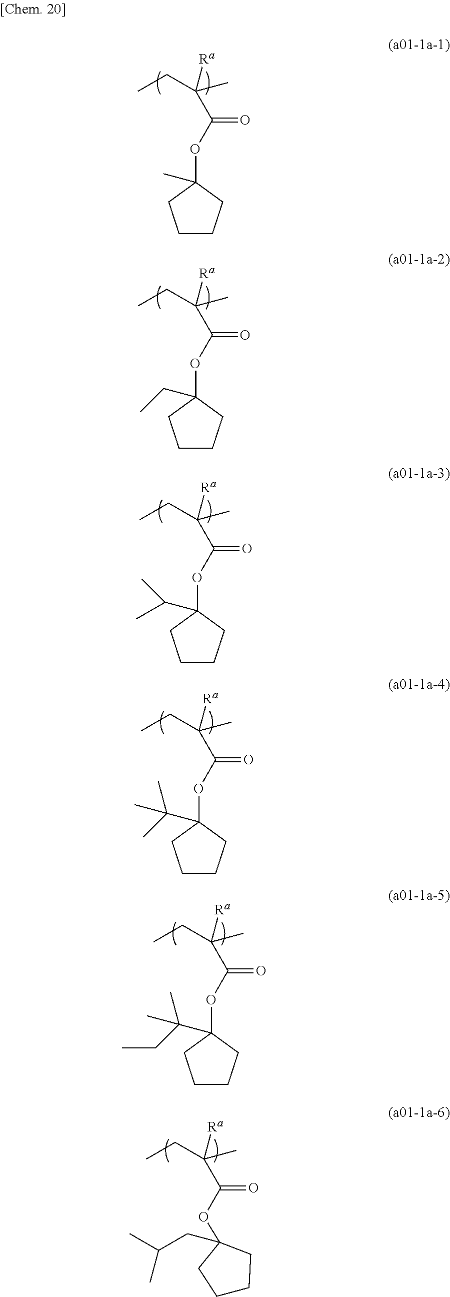

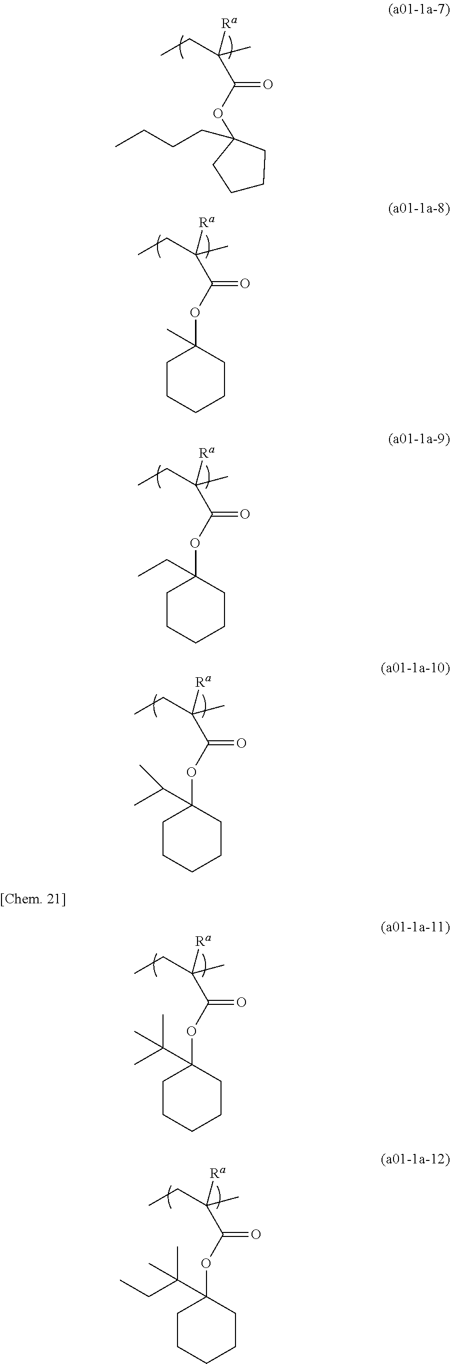

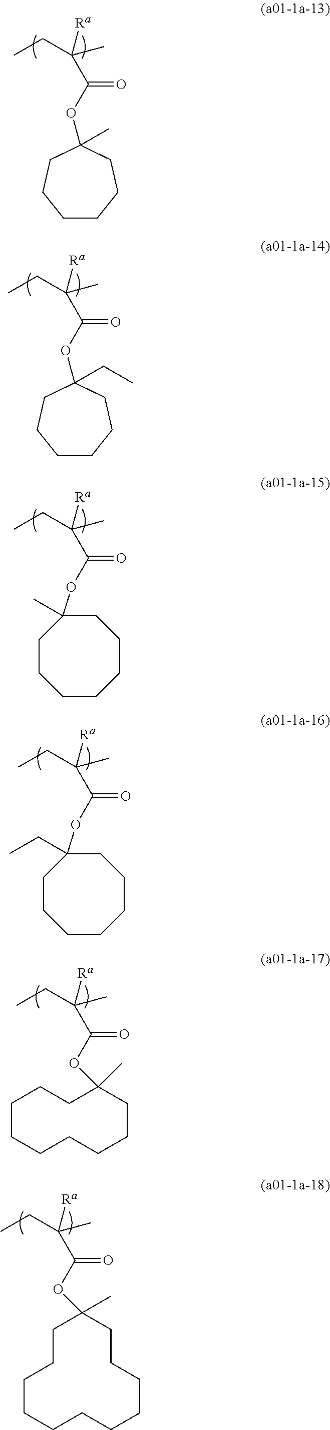

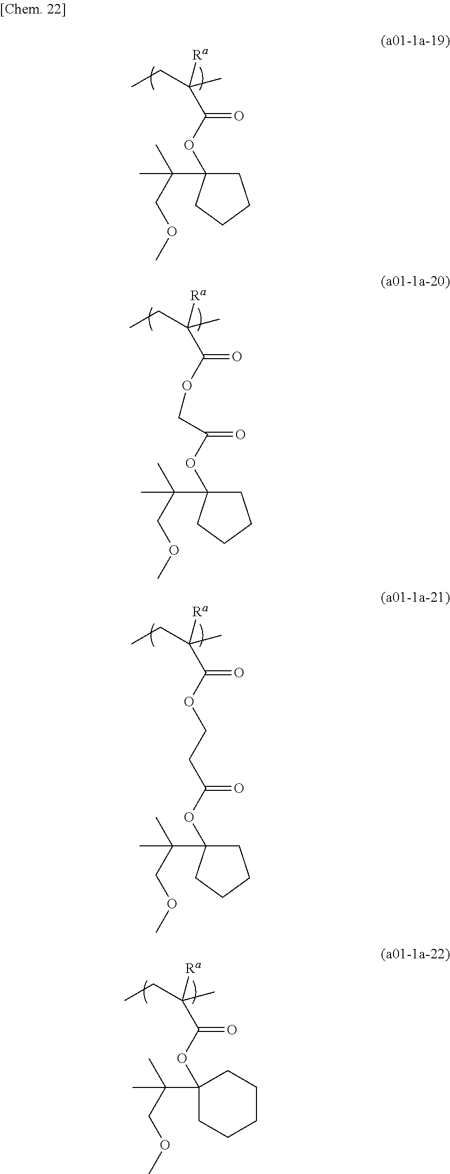

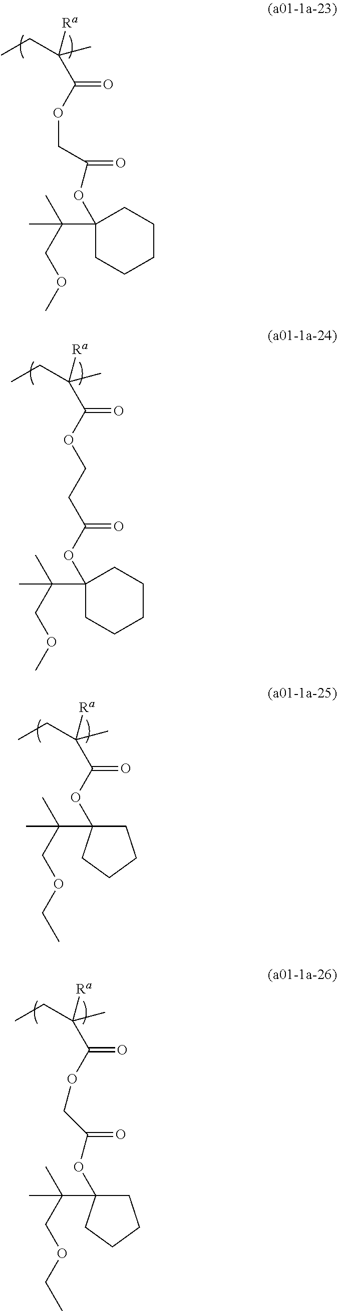

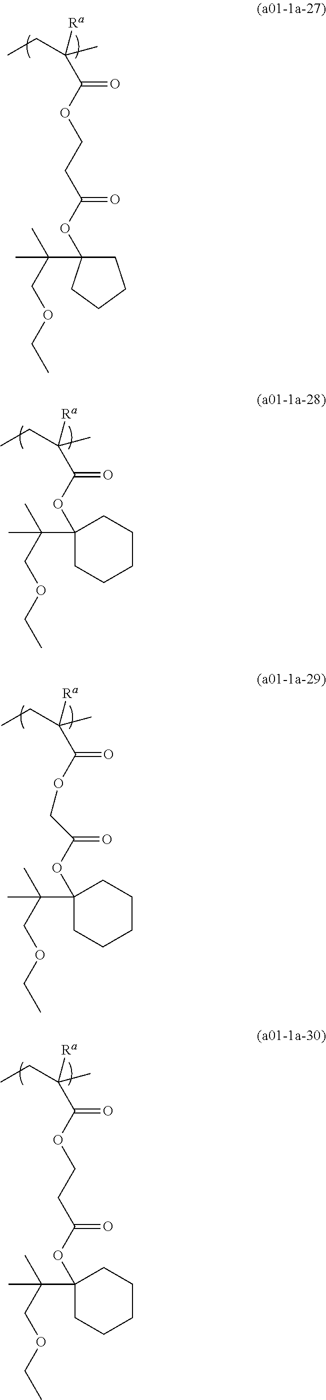

Among the examples, the structural unit (a1) is preferably at least one selected from the group consisting of structural units represented by chemical formulae (a01-1a-01) to (a01-1a-18), and more preferably at least one selected from the group consisting of structural units represented by chemical formulae (a01-1a-01) to (a01-1a-3), (a01-1a-5), (a01-1a-9), and (a01-1a-16).

The structural unit (a1) that the component (A1) may have may be one kind or two or more kinds thereof.

In the component (A1), a proportion of the structural unit (a1) is preferably 20 mol % to 80 mol %, more preferably 30 mol % to 70 mol %, and still more preferably 40 mol % to 60 mol %, with respect to the total (100 mol %) of all structural units constituting the component (A1).

When the proportion of the structural unit (a1) is equal to or larger than a lower limit value of the preferred range, lithography properties such as a high sensitivity, a high resolution, and roughness improvement are improved. When the proportion is equal to or less than an upper limit value of the preferred range, a good balance with other structural units can be achieved, and various lithography properties are improved.

(Structural Unit (a2))

The component (A1) may further include a structural unit (a2) including a lactone-containing cyclic group, a cyclic group containing —SO₂—, or a carbonate-containing cyclic group (excluding those corresponding to the structural unit (a1)).

The lactone-containing cyclic group, the —SO₂— containing cyclic group, or the carbonate-containing cyclic group of the structural unit (a2) is effective in enhancing adhesion of the resist film to the substrate when the component (A1) is used for forming a resist film. When the structural unit (a2) is contained, for example, an acid diffusion length can be appropriately adjusted, the adhesion of the resist film to the substrate can be enhanced, and the solubility during development can be appropriately adjusted, resulting in improved lithography properties.

The term “lactone-containing cyclic group” refers to a cyclic group containing a ring (a lactone ring) containing —O—C(═O)— in the ring skeleton thereof. When a lactone ring is counted as a first ring, a group having only a lactone ring is referred to as a monocyclic group, and a group further having another ring structure is referred to as a polycyclic group regardless of the structure. The lactone-containing cyclic group may be a monocyclic group or a polycyclic group.

The lactone-containing cyclic group in the structural unit (a2) is not particularly limited, and any group can be used. Specific examples thereof include a group represented by any one of the following general formulae (a2-r-1) to (a2-r-7).

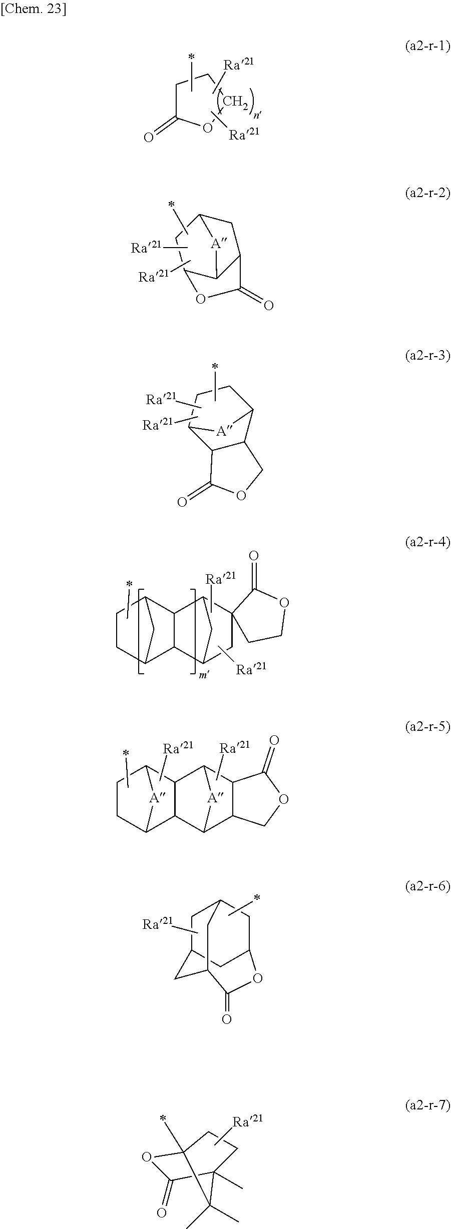

[In the general formulae (a2-r-1) to (a2-r-7), Ra′²¹'s each independently represent a hydrogen atom, an alkyl group, an alkoxy group, a halogen atom, a halogenated alkyl group, a hydroxy group, —COOR″, —OC(═O)R″, a hydroxyalkyl group, or a cyano group. R″ represents a hydrogen atom, an alkyl group, a lactone-containing cyclic group, a carbonate-containing cyclic group, or a —SO₂— containing cyclic group. A″ represents an alkylene group having 1 to 5 carbon atoms that may contain an oxygen atom (—O—) or a sulfur atom (—S—), an oxygen atom, or a sulfur atom; n′ is an integer of 0 to 2, and m′ is 0 or 1. * indicates a bond.]

In the general formulae (a2-r-1) to (a2-r-7), the alkyl group in Ra′²¹ is preferably an alkyl group having 1 to 6 carbon atoms. The alkyl group is preferably linear or branched. Specific examples thereof include a methyl group, an ethyl group, a propyl group, an iso-propyl group, an n-butyl group, an iso-butyl group, a tert-butyl group, a pentyl group, an iso-pentyl group, a neopentyl group, and a hexyl group. Among them, a methyl group or an ethyl group is preferred, and a methyl group is particularly preferred.

The alkoxy group in Ra′²¹ is preferably an alkoxy group having 1 to 6 carbon atoms. The alkoxy group is preferably linear or branched. Specific examples thereof include a group in which an oxygen atom (—O—) is linked to an alkyl group described as the alkyl group in Ra′²¹.

Examples of the halogen atom in Ra′²¹ include a fluorine atom, a chlorine atom, a bromine atom, and an iodine atom, and a fluorine atom is preferred.

Examples of the halogenated alkyl group in Ra′²¹ include a group in which a part or all of hydrogen atoms in the alkyl group in Ra′²¹ are replaced by the halogen atoms. The halogenated alkyl group is preferably a fluorinated alkyl group, and particularly preferably a perfluoroalkyl group.

In —COOR″ and —OC(═O)R″ in Ra′²¹, R″ represents a hydrogen atom, an alkyl group, a lactone-containing cyclic group, a carbonate-containing cyclic group, or a —SO₂— containing cyclic group.

The alkyl group in R″ may be linear, branched, or cyclic, and preferably has 1 to 15 carbon atoms.

When R″ is a linear or branched alkyl group, R″ preferably has 1 to 10 carbon atoms, and more preferably has 1 to 5 carbon atoms, and is particularly preferably a methyl group or an ethyl group.

When R″ is a cyclic alkyl group, R″ preferably has 3 to 15 carbon atoms, more preferably has 4 to 12 carbon atoms, and most preferably has 5 to 10 carbon atoms. Specific examples thereof include a group in which one or more hydrogen atoms are removed from a monocycloalkane that may or may not be replaced by a fluorine atom or a fluorinated alkyl group; and a group in which one or more hydrogen atoms are removed from a polycycloalkane such as a bicycloalkane, a tricycloalkane, or a tetracycloalkane. More specific examples thereof include a group in which one or more hydrogen atoms are removed from a monocycloalkane such as cyclopentane or cyclohexane; and a group in which one or more hydrogen atoms are removed from a polycycloalkane such as adamantane, norbomane, isobornane, tricyclodecane, or tetracyclododecane.

Examples of the lactone-containing cyclic group in R″ include those same as the group represented by any one of the general formulae (a2-r-1) to (a2-r-7).

The carbonate-containing cyclic group in R″ is the same as the carbonate-containing cyclic group described below, and specific examples thereof include a group represented by any one of general formulae (ax3-r-1) to (ax3-1-3).

The —SO₂— containing cyclic group in R″ is the same as the —SO₂— containing cyclic group described below, and specific examples thereof include a group represented by any one of general formulae (a5-r-1) to (a5-r-4).

The hydroxyalkyl group in Ra′²¹ preferably has 1 to 6 carbon atoms, and specific examples thereof include a group in which at least one hydrogen atom in the alkyl group in Ra′²¹ is replaced by a hydroxy group.

In the general formulae (a2-r-2), (a2-r-3), and (a2-r-5), the alkylene group having 1 to 5 carbon atoms in A″ is preferably a linear or branched alkylene group, and examples thereof include a methylene group, an ethylene group, an n-propylene group, and an isopropylene group. When the alkylene group contains an oxygen atom or a sulfur atom, specific examples thereof include a group in which —O— or —S— is interposed at the end of the alkylene group or between carbon atoms of the alkylene group, and examples thereof include —O—CH₂—, —CH₂—O—CH₂, —S—CH₂—, and —CH₂—S—CH₂—. A″ is preferably an alkylene group having 1 to 5 carbon atoms or —O—, more preferably an alkylene group having 1 to 5 carbon atoms, and most preferably a methylene group.

Specific examples of the group represented by any one of the general formulae (a2-r-1) to (a2-r-7) are shown below.

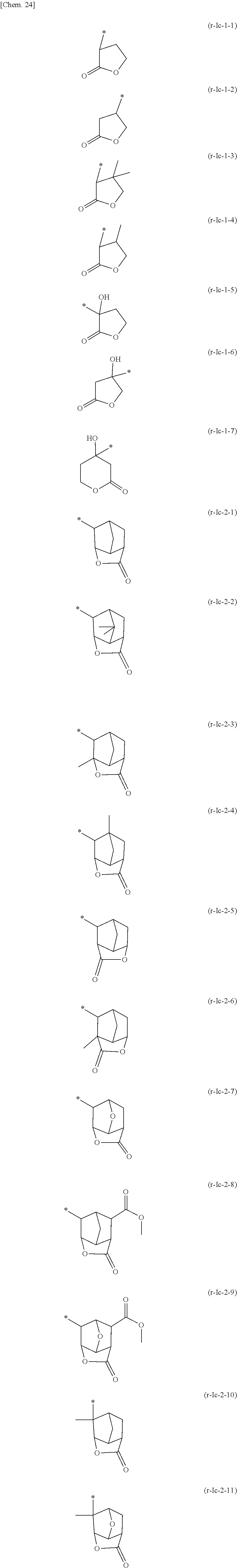

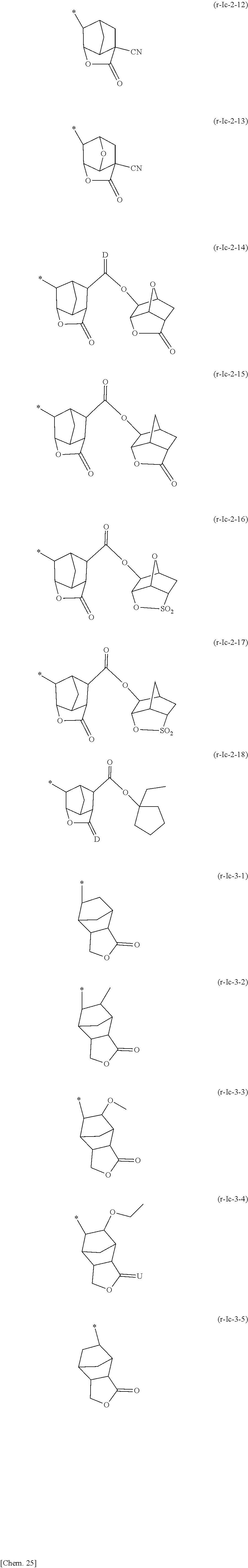

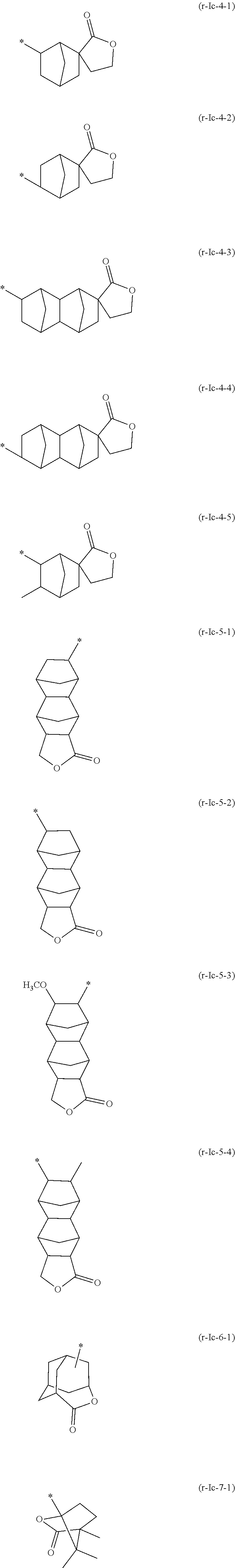

The term “—SO₂— containing cyclic group” refers to a cyclic group containing a ring containing —SO₂— in the ring skeleton thereof, and specifically, the —SO₂— containing cyclic group is a cyclic group in which a sulfur atom(S) in —SO₂— forms a part of the ring skeleton of the cyclic group. When a ring containing —SO₂— in the ring skeleton thereof is counted as a first ring, a group having only the ring is referred to as a monocyclic group, and a group further having another ring structure is referred to as a polycyclic group regardless of the structure. The —SO₂— containing cyclic group may be a monocyclic group or a polycyclic group.

The —SO₂— containing cyclic group is particularly preferably a cyclic group containing —O—SO₂— in the ring skeleton thereof, that is, a cyclic group containing a sultone ring in which —O—S— in —O—SO₂— forms a part of the ring skeleton.

Specific examples of the cyclic group containing —SO₂— include groups each represented by the following general formulae (a5-r-1) to (a5-r-4).

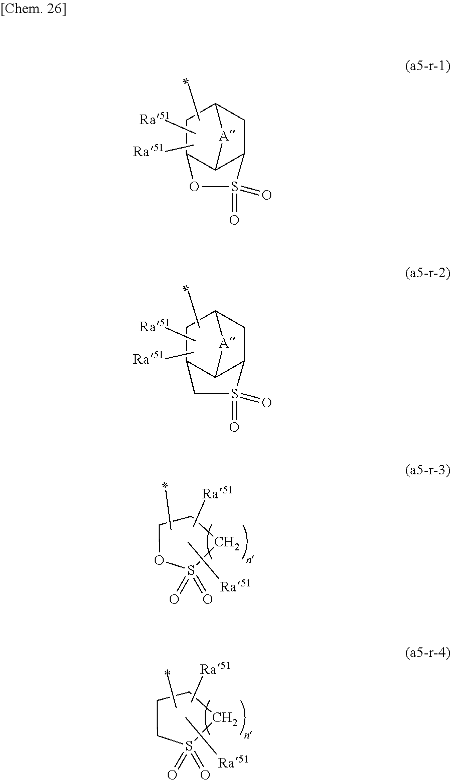

[In the general formulae (a5-r-1) to (a5-1-4), Ra′⁵¹'s each independently represent a hydrogen atom, an alkyl group, an alkoxy group, a halogen atom, a halogenated alkyl group, a hydroxy group, —COOR″, —OC(═O) R″, a hydroxyalkyl group, or a cyano group. R″ represents a hydrogen atom, an alkyl group, a lactone-containing cyclic group, a carbonate-containing cyclic group, or a —SO₂— containing cyclic group. A″ represents an alkylene group having 1 to 5 carbon atoms which may contain an oxygen atom or a sulfur atom, an oxygen atom, or a sulfur atom, and n′ is an integer of 0 to 2.]

In the general formulae (a5-r-1) and (a5-r-2), A″ is the same as A″ in the general formulae (a2-r-2), (a2-r-3) and (a2-r-5).

Examples of the alkyl group, alkoxy group, halogen atom, halogenated alkyl group, —COOR″, —OC(═O) R″, and hydroxyalkyl group in Ra′⁵¹ include those same as those described in the description of Ra′²¹ in the general formulae (a2-r-1) to (a2-r-7).

Specific examples of the group represented by any one of the general formulae (a5-r-1) to (a5-r-4) are shown below. In the formula, “Ac” represents an acetyl group.

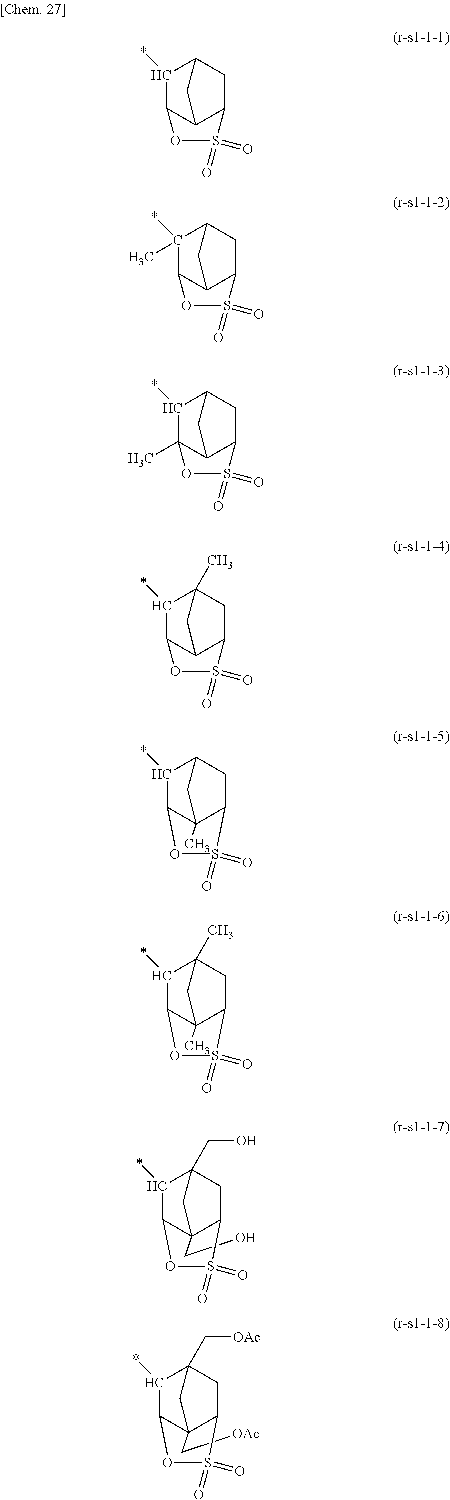

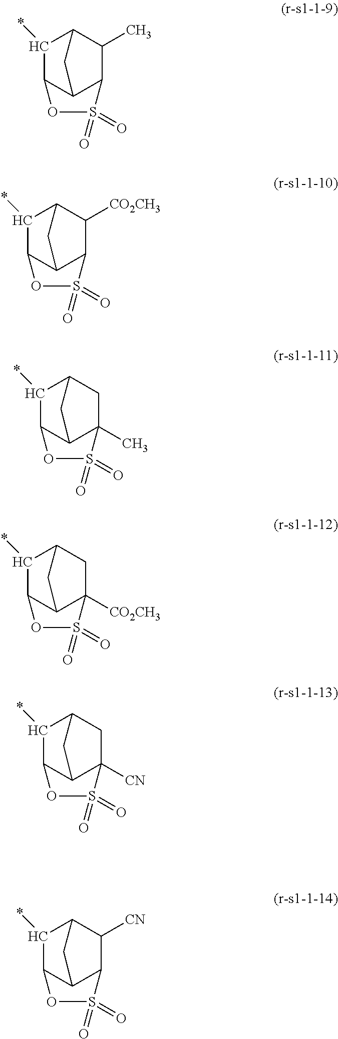

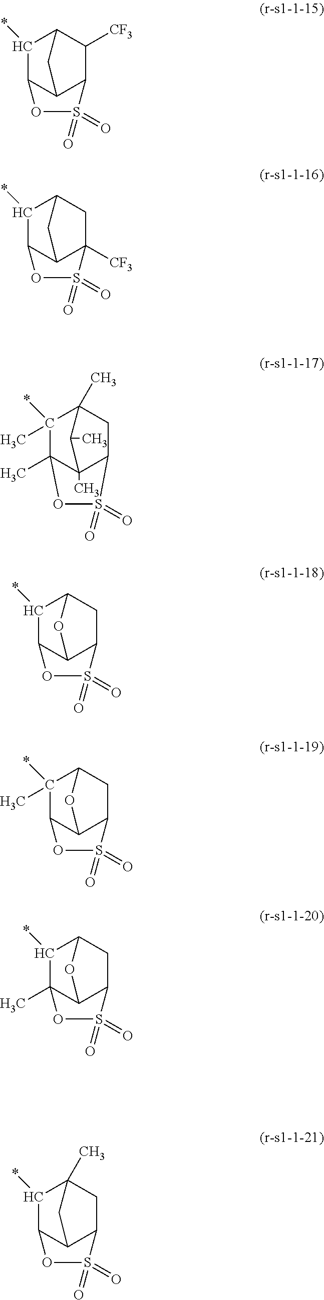

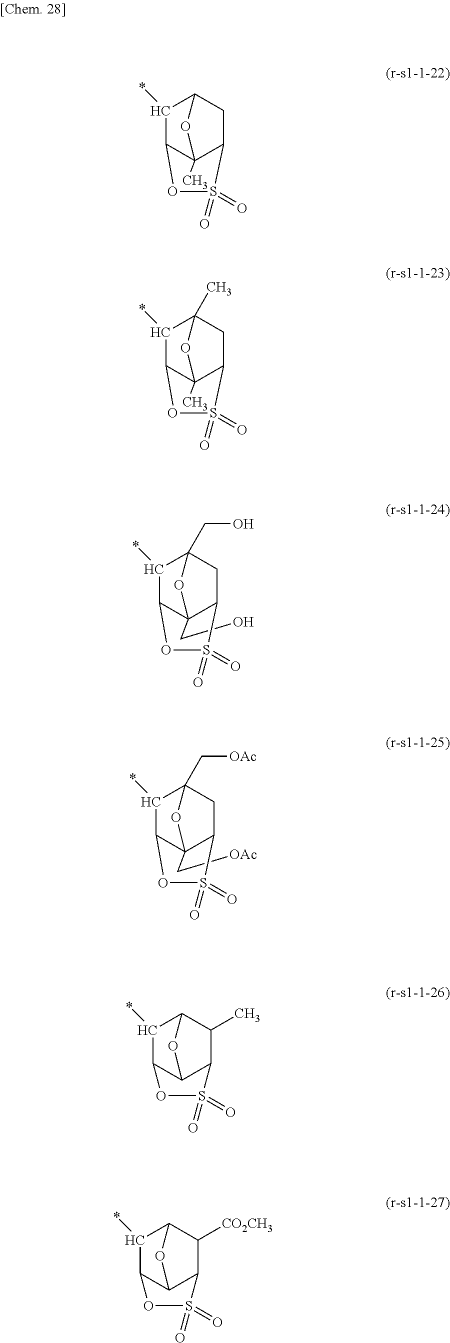

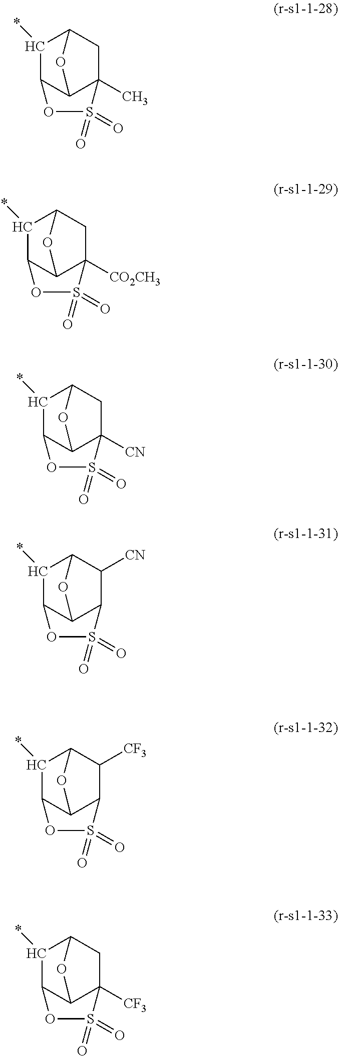

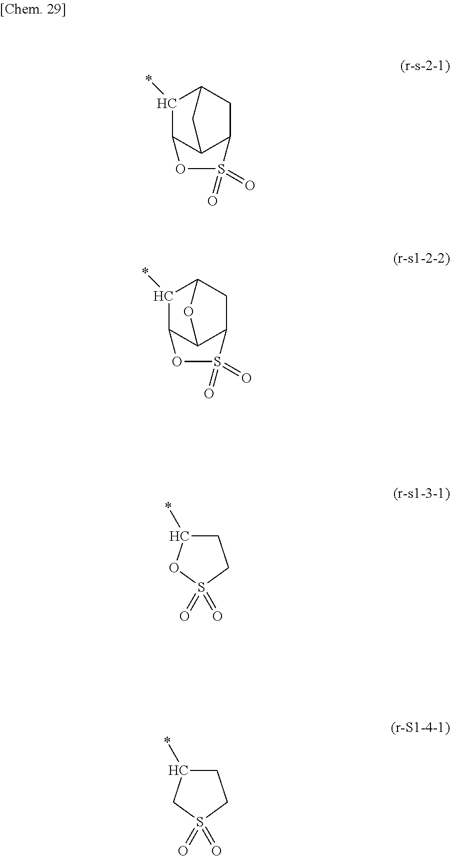

The term “carbonate-containing cyclic group” refers to a cyclic group containing a ring (a carbonate ring) containing —O—C(═O)—O— in the ring skeleton thereof. When a carbonate ring is counted as a first ring, a group having only a carbonate ring is referred to as a monocyclic group, and a group further having another ring structure is referred to as a polycyclic group regardless of the structure. The carbonate-containing cyclic group may be a monocyclic group or a polycyclic group.

The carbonate ring-containing cyclic group is not particularly limited, and any group can be used. Specific examples thereof include a group represented by any one of the following general formulae (ax3-r-1) to (ax3-1-3).

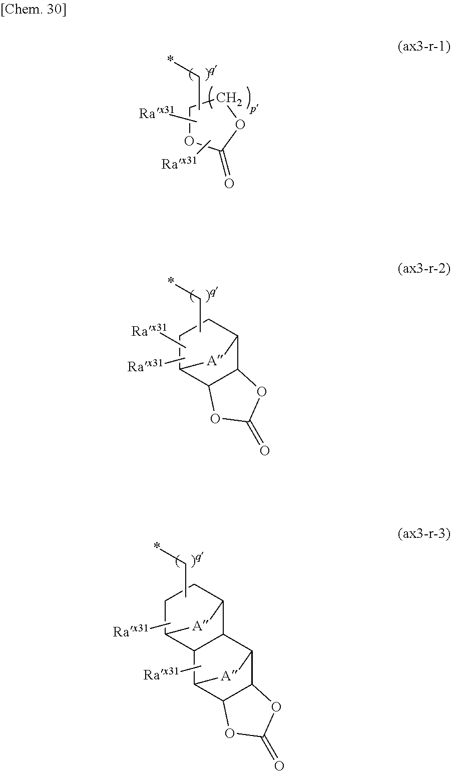

[In the formulae, Ra′³¹'s each independently represent a hydrogen atom, an alkyl group, an alkoxy group, a halogen atom, a halogenated alkyl group, a hydroxy group, —COOR″, —OC(═O)R″, a hydroxyalkyl group, or a cyano group. R″ represents a hydrogen atom, an alkyl group, a lactone-containing cyclic group, a carbonate-containing cyclic group, or a —SO₂— containing cyclic group. A″ represents an alkylene group having 1 to 5 carbon atoms that may contain an oxygen atom or a sulfur atom, an oxygen atom, or a sulfur atom. p′ is an integer of 0 to 3, and q′ is 0 or 1. * indicates a bond.]

In the general formulae (ax3-r-2) to (ax3-1-3), A″ is the same as A″ in the general formulae (a2-r-2), (a2-r-3) and (a2-r-5).

Examples of the alkyl group, alkoxy group, halogen atom, halogenated alkyl group, —COOR″, —OC(═O) R″, and hydroxyalkyl group in Ra′^{x31} include those same as those described in the description of Ra′²¹ in the general formulae (a2-r-1) to (a2-r-7).

Specific examples of the group represented by any one of the general formulae (ax3-r-1) to (ax3-r-3) are shown below.

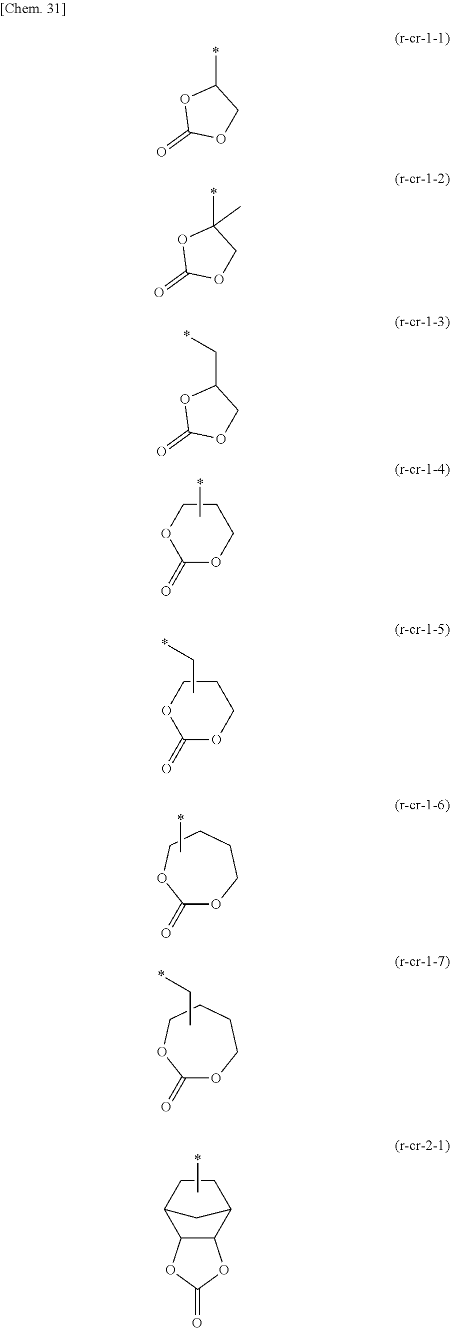

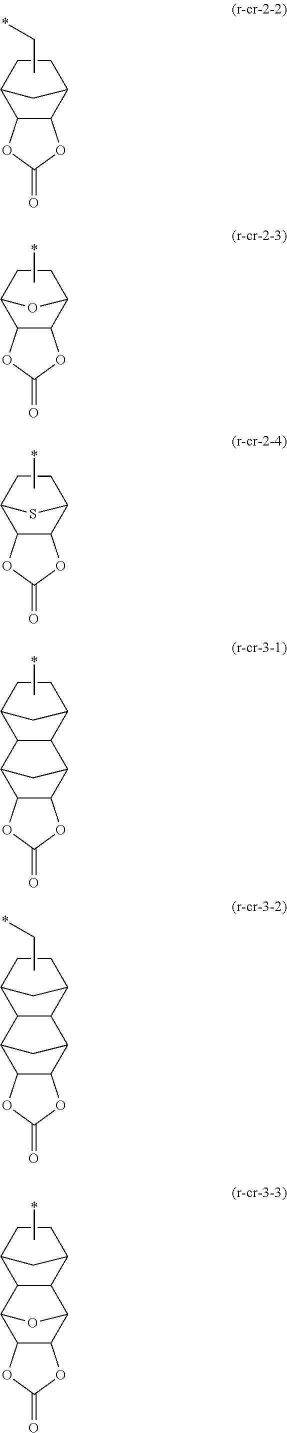

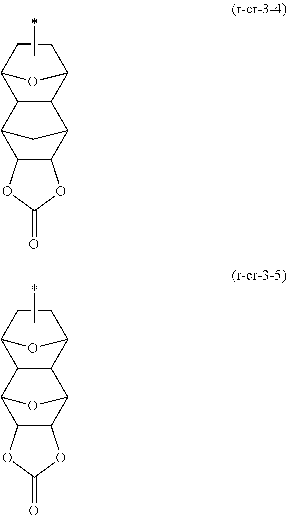

As the structural unit (a2), a structural unit derived from an acrylic ester in which a hydrogen atom bonded to a carbon atom at an q-position may be replaced by a substituent is preferred.

Such a structural unit (a2) is preferably a structural unit represented by the following general formula (a2-1).

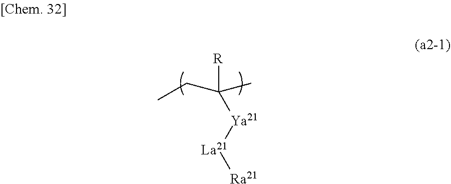

(In the general formula (a2-1), R represents a hydrogen atom, an alkyl group having 1 to 5 carbon atoms, or a halogenated alkyl group having 1 to 5 carbon atoms. Ya²¹ represents a single bond or a divalent linking group. La²¹ is —O—, —COO—, —CON(R′)—, —OCO—, —CONHCO— or —CONHCS—, R′ represents a hydrogen atom or a methyl group. When La²¹ is —O—, Ya²¹ does not represent —CO—. Ra²¹ is a lactone-containing cyclic group, a carbonate-containing cyclic group, or a —SO₂— containing cyclic group.]

In the formula (a2-1), R is the same as R in the formula (a1-1). R is preferably a hydrogen atom, an alkyl group having 1 to 5 carbon atoms, or a fluorinated alkyl group having 1 to 5 carbon atoms, and from the viewpoint of industrial availability, a hydrogen atom or a methyl group is particularly preferred.

In the formula (a2-1), the divalent linking group in Ya²¹ is not particularly limited, and suitable examples thereof include a divalent hydrocarbon group that may include a substituent, and a divalent linking group containing a heteroatom.

Divalent Hydrocarbon Group that May Include Substituent:

When Ya²¹ is a divalent hydrocarbon group that may include a substituent, the hydrocarbon group may be an aliphatic hydrocarbon group or an aromatic hydrocarbon group.

### Aliphatic Hydrocarbon Group in Ya²¹

An aliphatic hydrocarbon group means a hydrocarbon group having no aromaticity. The aliphatic hydrocarbon group may be saturated or unsaturated, and is generally preferably saturated.

Examples of the aliphatic hydrocarbon group include a linear or branched aliphatic hydrocarbon group and an aliphatic hydrocarbon group containing a ring in the structure thereof.

### Linear or Branched Aliphatic Hydrocarbon Group

The linear aliphatic hydrocarbon group preferably has 1 to 10 carbon atoms, more preferably has 1 to 6 carbon atoms, still more preferably has 1 to 4 carbon atoms, and most preferably has 1 to 3 carbon atoms.

The linear aliphatic hydrocarbon group is preferably a linear alkylene group, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group preferably has 2 to 10 carbon atoms, more preferably has 3 to 6 carbon atoms, still more preferably has 3 or 4 carbon atoms, and most preferably has 3 carbon atoms.

The branched aliphatic hydrocarbon group is preferably a branched alkylene group, and specific examples thereof include alkyl alkylene groups such as alkylmethylene groups such as —CH(CH₃)—, —CH(CH₂CH₃)₂—, —C(CH₃)₂—, —C(CH₃)(CH₂CH₃)—, —C(CH₃)(CH₂CH₂CH₃)—, and —C(CH₂CH₃)₂—; alkyl ethylene groups such as —CH(CH₃)CH₂—, —CH(CH₃)CH(CH₃)—, —C(CH₃)₂CH₂—, —CH(CH₂CH₃)CH₂—, and —C(CH₂CH₃)CH₂—, alkyl trimethylene groups such as —CH(CH₃)CH₂CH₂— and —CH₂CH(CH₃)CH₂—; and alkyl tetramethylene groups such as —CH(CH₃)CH₂CH₂CH₂— and —CH₂CH(CH₃)CH₂CH₂—. The alkyl group in the alkylalkylene group is preferably a linear alkyl group having 1 to 5 carbon atoms.

The linear or branched aliphatic hydrocarbon group may or may not include a substituent. Examples of the substituent include a fluorine atom, a fluorinated alkyl group having 1 to 5 carbon atoms and being replaced by a fluorine atom, and a carbonyl group.

### Aliphatic Hydrocarbon Group Containing Ring in Structure Thereof

Examples of the aliphatic hydrocarbon group containing a ring in the structure thereof include a cyclic aliphatic hydrocarbon group that may include a substituent containing a heteroatom in the ring structure thereof (a group in which two hydrogen atoms are removed from an aliphatic hydrocarbon ring), a group in which the cyclic aliphatic hydrocarbon group is bonded to the end of a linear or branched aliphatic hydrocarbon group, and a group in which the cyclic aliphatic hydrocarbon group is interposed in the middle of a linear or branched aliphatic hydrocarbon group. Examples of the linear or branched aliphatic hydrocarbon group include those same as described above.

The cyclic aliphatic hydrocarbon group preferably has 3 to 20 carbon atoms, and more preferably has 3 to 12 carbon atoms.

The cyclic aliphatic hydrocarbon group may be either a polycyclic group or a monocyclic group. The monocyclic alicyclic hydrocarbon group is preferably a group in which two hydrogen atoms are removed from a monocycloalkane. The monocycloalkane preferably has 3 to 6 carbon atoms, and specific examples thereof include cyclopentane and cyclohexane. The polycyclic alicyclic hydrocarbon group is preferably a group in which two hydrogen atoms are removed from a polycycloalkane. The polycycloalkane is preferably a group having 7 to 12 carbon atoms, and specific examples thereof include adamantane, norbomane, isobornane, tricyclodecane, and tetracyclododecane.

The cyclic aliphatic hydrocarbon group may or may not include a substituent. Examples of the substituent include an alkyl group, an alkoxy group, a halogen atom, a halogenated alkyl group, a hydroxy group, and a carbonyl group.

The alkyl group as the substituent is preferably an alkyl group having 1 to 5 carbon atoms, and more preferably a methyl group, an ethyl group, a propyl group, an n-butyl group, or a tert-butyl group.

The alkoxy group as the substituent is preferably an alkoxy group having 1 to 5 carbon atoms, more preferably a methoxy group, an ethoxy group, an n-propoxy group, an iso-propoxy group, an n-butoxy group, or a tert-butoxy group, and still more preferably a methoxy group, or an ethoxy group.

Examples of the halogen atom as the substituent include a fluorine atom, a chlorine atom, a bromine atom, and an iodine atom. A fluorine atom is preferred.

Examples of the halogenated alkyl group as the substituent include a group in which a part or all of hydrogen atoms of the alkyl group are replaced by halogen atoms.

In the cyclic aliphatic hydrocarbon group, a part of carbon atoms constituting the ring structure thereof may be replaced by a substituent containing a heteroatom. The substituent containing a heteroatom is preferably —O—, —C(═O)—O—, —S—, —S(═O)₂, or —S(═O)—O.

### Aromatic Hydrocarbon Group in Ya²¹

The aromatic hydrocarbon group is a hydrocarbon group having at least one aromatic ring.

The aromatic ring is not particularly limited as long as it is of a cyclic conjugated system having 4n+2π electrons, and may be monocyclic or polycyclic. The number of carbon atoms of the aromatic ring is preferably 5 to 30, more preferably 5 to 20, still more preferably 6 to 15, and particularly preferably 6 to 12. The number of the carbon atoms does not include the number of the carbon atoms in the substituent.

Specific examples of the aromatic ring include an aromatic hydrocarbon ring such as benzene, naphthalene, anthracene, and phenanthrene; and an aromatic heterocyclic ring in which a part of carbon atoms constituting the aromatic hydrocarbon ring is replaced by a heteroatom. Examples of the heteroatom in the aromatic heterocyclic ring include an oxygen atom, a sulfur atom, and a nitrogen atom. Specific examples of the aromatic heterocyclic ring include a pyridine ring and a thiophene ring.

Specific examples of the aromatic hydrocarbon group include a group in which two hydrogen atoms are removed from the aromatic hydrocarbon ring or aromatic heterocyclic ring (an arylene group or a heteroarylene group), a group in which two hydrogen atoms are removed from an aromatic compound containing two or more aromatic rings (for example, biphenyl or fluorene); and a group in which one hydrogen atom of a group in which one hydrogen atom is removed from the aromatic hydrocarbon ring or aromatic heterocyclic ring (an aryl group or a heteroaryl group) is replaced by an alkylene group (for example, a group in which one hydrogen atom is further removed from the aryl group in the arylalkyl group, such as a benzyl group, a phenethyl group, a 1-naphthylmethyl group, a 2-naphthylmethyl group, a 1-naphthylethyl group, or a 2-naphthylethyl group). The number of carbon atoms of the alkylene group bonded to the aryl group or the heteroaryl group is preferably 1 to 4, more preferably 1 to 2, and particularly preferably 1.

In the aromatic hydrocarbon group, a hydrogen atom contained in the aromatic hydrocarbon group may be replaced by a substituent. For example, a hydrogen atom bonded to an aromatic ring in the aromatic hydrocarbon group may be replaced by a substituent. Examples of the substituent include an alkyl group, an alkoxy group, a halogen atom, a halogenated alkyl group, and a hydroxy group.

The alkyl group as the substituent is preferably an alkyl group having 1 to 5 carbon atoms, and more preferably a methyl group, an ethyl group, a propyl group, an n-butyl group, or a tert-butyl group.

Examples of the alkoxy group, halogen atom, and halogenated alkyl group as the substituent include those exemplified as the substituent for replacing the hydrogen atom in the cyclic aliphatic hydrocarbon group.

### Divalent Linking Group Containing Heteroatom:

When Ya²¹ is a divalent linking group containing a heteroatom, preferred examples of the linking group include —O—, —C(═O)—O—, —O—C(═O)—, —C(═O)—, —O—C(═O)—O—, —C(═O)—NH—, —NH—, —NH—C(═NH)— (H may be replaced by a substituent such as an alkyl group or an acyl group), —S—, —S(═O)₂—, —S(═O)₂—O—, and a group represented by a formula —Y²¹—O—Y²², —Y²¹—O—, —Y²¹—C(═O)—O—, —C(═O)—O—Y²¹—, —[Y²¹—C(═O)—O]ₘ—Y²¹, —Y²¹—O—C(═O)—Y²²— or —Y²¹—S(═O)₂—O—Y²²— [in the formulae, Y²¹ and Y²² each independently represent a divalent hydrocarbon group that may include a substituent, O is an oxygen atom, and m″ is an integer of 0 to 3].

When the divalent linking group containing a heteroatom is —C(═O)—NH—, —NH—, or —NH—C(═NH)—, H thereof may be replaced by a substituent such as an alkyl group or an acyl group. The substituent (an alkyl group, an acyl group, or the like) preferably has 1 to 10 carbon atoms, more preferably has 1 to 8 carbon atoms, and particularly preferably has 1 to 5 carbon atoms.

In the formulae. Y²¹—O—Y²², Y²¹—O—, —Y²¹)—C(═O)—O, —C(═O)—O—Y²¹—, —[Y²¹—C(═O)—O]ₘ—Y²², —Y²¹—O—C(═O)—Y²²— or —Y²¹—S(═O)₂—O—Y²²—, Y²¹ and Y²² each independently represent a divalent hydrocarbon group that may include a substituent. Examples of the divalent hydrocarbon group include those same as those described above for the divalent linking group in Ya²¹ (the divalent hydrocarbon group that may include a substituent).

Y²¹ is preferably a linear aliphatic hydrocarbon group, more preferably a linear alkylene group, still more preferably a linear alkylene group having 1 to 5 carbon atoms, and particularly preferably a methylene group or an ethylene group.

Y²² is preferably a linear or branched aliphatic hydrocarbon group, and more preferably a methylene group, an ethylene group, or an alkylmethylene group. The alkyl group in the alkylmethylene group is preferably a linear alkyl group having 1 to 5 carbon atoms, more preferably a linear alkyl group having 1 to 3 carbon atoms, and most preferably a methyl group.

In the group represented by the formula —[Y²¹—C(═O)—O]_{m″}—Y²²—, m″ is an integer of 0 to 3, preferably an integer of 0 to 2, more preferably 0 or 1, and particularly preferably 1. That is, the group represented by the formula —[Y²¹—C(═O)—O]_{m″}—Y²²—, a group represented by the formula —Y²¹—C(═O)—O—Y²²— is particularly preferred. Among them, a group represented by the formula —(CH₂)ₐ—C(═O)—O—(CH₂)_{b}— is preferred. In the formula, a′ is an integer of 1 to 10, preferably an integer of 1 to 8, more preferably an integer of 1 to 5, still more preferably 1 or 2, and most preferably 1. The symbol b′ is an integer of 1 to 10, preferably an integer of 1 to 8, more preferably an integer of 1 to 5, still more preferably 1 or 2, and most preferably 1.

Among them, Ya²¹ is preferably a single bond, an ester bond [—C(═O)—O—], an ether bond (—O—), a linear or branched alkylene group, or a combination thereof.

In the formula (a2-1), Ra²¹ represents a lactone-containing cyclic group, a —SO₂— containing cyclic group, or a carbonate-containing cyclic group.

Suitable examples of the lactone-containing cyclic group, the —SO₂— containing cyclic group, and the carbonate-containing cyclic group in Ra²¹ include a group represented by any one of the general formulae (a2-r-1) to (a2-r-7), a group represented by any one of general formulae (a5-r-1) to (a5-r-4), and a group represented by any one of general formulae (ax3-r-1) to (ax3-1-3).

In the formula (a2-1), among the above, Ra²¹ is preferably a lactone-containing cyclic group or a —SO₂— containing cyclic group, more preferably a group represented by the general formula (a2-r-1), (a2-r-2), (a2-r-6), or (a5-r-1), and still more preferably a group represented by any one of the general formulae (a2-r-1) and (a2-r-2).

Specifically, any one of the groups represented by the chemical formulae (r-1c-1-1) to (r-1c-1-7), (r-1c-2-1) to (r-1c-2-18), and (r-1c-6-1) is preferred, and any one of the groups represented by the chemical formula (r-1c-1-1), (r-1c-2-1), and (r-1c-2-12) is more preferred.

The structural unit (a2) contained in the component (A1) may be used alone or in combination of two or more kinds thereof.

When the component (A1) contains the structural unit (a2), a proportion of the structural unit (a2) is preferably 20 mol % to 80 mol %, more preferably 30 mol % to 70 mol %, and particularly preferably 40 mol % to 60 mol %, with respect to the total (100 mol %) of all structural units constituting the component (A1).

When the proportion of the structural unit (a2) is equal to or larger than a preferred lower limit value, the effects of containing the structural unit (a2) can be sufficiently obtained due to the above-described effects. When the proportion of the structural unit (a2) is equal to or less than an upper limit value, a balance with other structural units can be achieved, and various lithography properties become excellent.

### <<Other Structural Units>>

The component (A1) may have other structural units in addition to the structural unit (a1) and structural unit (a2).

Examples of the other structural units include a structural unit (a3) containing a polar group-containing aliphatic hydrocarbon group, a structural unit (a6) containing a hydroxystyrene skeleton, and a structural unit (a8) containing an acid-indissociable aliphatic cyclic group. As the structural units (a3) to (a8), a large number of the related-art known structural units used for a resin component of a resist composition can be used except for structural units corresponding to the structural unit (a1) or the structural unit (a2).

A proportion of the component (A1) in the component (A) is preferably 25 mass % or more, more preferably 50 mass % or more, still more preferably 75 mass % or more, and may be 100 mass %, with respect to the total mass of the component (A). When the proportion is 25 mass % or more, a resist pattern excellent in various lithography properties such as a high sensitivity, a resolution, and roughness is easily formed.

In the resist composition according to the embodiment of the present invention, a content of the component (A) may be adjusted according to a thickness of the resist film to be formed.

### <<Acid Generating Agent Component (Component (B))>>

The resist composition according to the embodiment of the present invention contains, in addition to the component (A), the acid generating agent component (B) (hereinafter, also referred to as “component (B)”) that generates an acid upon exposure, and the acid generating agent component (B) contains an acid generating agent (B1) (hereinafter, also referred to as “component (B1)”) composed of a compound represented by the following general formula (b1-1).

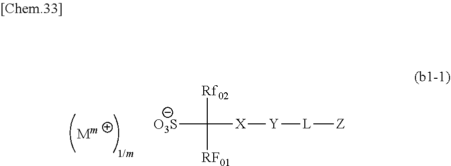

[In the general formula (b1-1), Rf₀₁ and Rf₀₂ each independently represent a hydrogen atom, a fluorine atom, or a fluorinated alkyl group, and at least one of Rf₀₁ and Rf₀₂ contains a fluorine atom. X represents a divalent linking group having a total of 4 or more atoms in the main chain, and the divalent linking group represents an alkylene group which may contain an ester bond or an ether bond, a fluorinated alkylene group which may contain an ester bond or an ether bond, or a combination thereof. Any of ester bonds and ether bonds in X does not form an acetal bond adjacent to each other. Y represents a divalent polycyclic hydrocarbon group that may include a substituent. L represents a divalent linking group containing an ester bond or an oxygen atom, and a plurality of ester bonds and a plurality of oxygen atoms may be each bonded independently via an alkylene group. Z represents a substituent represented by the following formula (bz-1), m represents an integer of 1 or more, and M^{m+} represents an m-valent organic cation.]

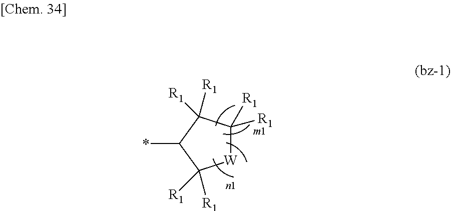

[In the formula (bz-1), R₁'s each independently represent a hydrogen atom, a halogen atom, a linear or branched alkyl group in which a part of carbon atoms may be replaced by a heteroatom, a linear or branched acyl group in which a part of carbon atoms may be replaced by a heteroatom, or a linear or branched alkyloxycarbonyl group in which a part of carbon atoms may be replaced by a heteroatom, W represents an oxygen atom, a sulfur atom, or —NRⁿ—, Rⁿ represents a hydrogen atom, or a linear or branched alkyl group, m1 and n1 each represent an integer of 0 to 4, and m1+n1 represents an integer of 0 to 4, and * represents a bond.]

[Anion Moiety (Z-L-Y—C(Rf₀₁)(Rf₀₂)—SO₃⁻)]

In the general formula (b1-1), Rf₀₁ and Rf₀₂ each independently represent a hydrogen atom, a fluorine atom, or a fluorinated alkyl group, and at least one of Rf₀₁ and Rf₀₂ contains a fluorine atom.

The fluorinated alkyl groups represented by Rf₀₁ and Rf₀₂ may each independently be chained or cyclic, and are preferably linear or branched. The number of carbon atoms of the fluorinated alkyl group is preferably 1 to 11, more preferably 1 to 8, and still more preferably 1 to 4.

Specific examples thereof include a group in which a part or all of hydrogen atoms constituting a linear alkyl group, such as a methyl group, an ethyl group, a propyl group, a butyl group, a pentyl group, a hexyl group, a heptyl group, an octyl group, a nonyl group or a decyl group are replaced by fluorine atoms; and a group in which a part or all of hydrogen atoms constituting a branched alkyl group, such as a 1-methylethyl group, a 1-methylpropyl group, a 2-methylpropyl group, a 1-methylbutyl group, a 2-methylbutyl group or a 3-methylbutyl group are replaced by fluorine atoms.

The fluorinated alkyl groups represented by Rf₀₁ and Rf₀₂ may each independently contain an atom other than a fluorine atom, a carbon atom and a hydrogen atom, for example, an oxygen atom, a sulfur atom, and a nitrogen atom.

Among them, the fluorinated alkyl group represented by Rf₀₁ and Rf₀₂ is preferably a group in which a part or all of hydrogen atoms constituting the linear alkyl group are replaced by fluorine atoms, and is preferably a group in which all of hydrogen atoms constituting the linear alkyl group are replaced by fluorine atoms (a perfluoroalkyl group).

In the general formula (b1-1), Rf₀₁ and Rf₀₂ each preferably represent a fluorine atom.

In the general formula (b1-1), X represents a divalent linking group having a total of 4 or more atoms in the main chain, and the divalent linking group represents an alkylene group which may contain an ester bond or an ether bond, a fluorinated alkylene group which may contain an ester bond or an ether bond, or a combination thereof. Any of ester bonds and ether bonds in X does not form an acetal bond adjacent to each other.

Examples of the ester bond include —O—C(═O)— and —C(═O) O.

As the divalent linking group represented by X and having a total of 4 or more atoms in the main chain, any one of the linking groups represented by the following formulae (y-b1-1) to (y-b1-12) is more preferred.

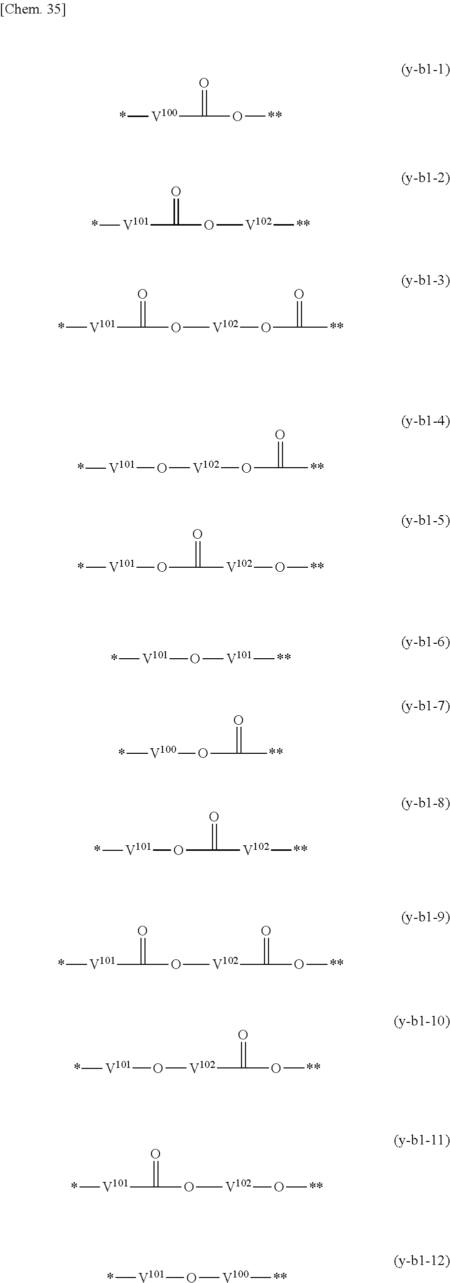

[In formulae (y-b1-1) to (y-b1-12), V¹⁰⁰ represents an alkylene group having 2 to 5 carbon atoms or a fluorinated alkylene group having 2 to 5 carbon atoms, V¹⁰¹ represents a single bond, an alkylene group having 1 to 5 carbon atoms or a fluorinated alkylene group having 1 to 5 carbon atoms, V¹⁰² represents an alkylene group having 2 to 30 carbon atoms or a fluorinated alkylene group having 2 to 30 carbon atoms, and the total number of atoms in the main chain in the formulae (y-b1-1) to (y-b1-12) is 4 or more. The symbol * represents a bond to be bonded to the carbon atom to which Rf₀₁ and Rf₀₂ are bonded, and ** represents a bond to be bonded to Y.]

In the formulae (y-b1-1) to (y-b1-12), the alkylene group in V¹⁰⁰, viol, and V¹⁰² may be linear or branched.

As the alkylene group having 2 to 5 carbon atoms in V¹⁰⁰, an alkylene group having 2 to 4 carbon atoms is preferred, and an alkylene group having 2 to 3 carbon atoms is more preferred.

Specific examples thereof include an ethylene group [—CH₂CH₂—]; an alkyl ethylene group such as —CH(CH₃)CH₂—, —CH(CH₃)CH(CH₃)—, —C(CH₃)₂CH₂—, and —CH(CH₂CH₃)CH₂—; a trimethylene group (an n-propylene group) [—CH₂CH₂CH₂—]; an alkyl trimethylene group such as —CH(CH₃)CH₂CH₂— and —CH₂CH(CH₃)CH₂—; a tetramethylene group [—CH₂CH₂CH₂CH₂—]; an alkyl tetramethylene group such as —CH(CH₃)CH₂CH₂CH₂— and —CH₂CH(CH₃)CH₂CH₂—; and a pentamethylene group [—CH₂CH₂CH₂CH₂CH₂—].

As the fluorinated alkylene group having 2 to 5 carbon atoms in V¹⁰⁰, a fluorinated alkylene group having 2 to 4 carbon atoms is preferred, and a fluorinated alkylene group having 2 to 3 carbon atoms is more preferred.

Examples of the fluorinated alkylene group having 2 to 5 carbon atoms in V¹⁰⁰ include a group in which a part or all of hydrogen atoms in the alkylene group having 2 to 5 carbon atoms in V¹⁰⁰ are replaced by fluorine atoms, and a linear fluorinated alkylene group having 2 to 5 carbon atoms is preferred.

Specific examples thereof include a fluorinated ethylene group [—CHFCH₂—, —CF₂CH₂—]; a fluorinated alkyl ethylene group such as —CH(CF₃)CH₂—, —CH(CF₃)CH(CH₃)—, and —C(CF₃)CH₂—; a fluorinated trimethylene group (a fluorinated n-propylene group) [—CHFCH₂CH₂, —CF₂CH₂CH₂]; a fluorinated alkyl trimethylene group such as —CH(CF₃)CH₂CH₂—, and —CHFCH(CH₃)CH₂—; a fluorinated tetramethylene group [—CHFCH₂CH₂CH₂—, and —CF₂CH₂CH₂CH₂—]; a fluorinated alkyl tetramethylene group such as —CH(CF₃)CH₂CH₂CH₂—, and —CHFCH(CH₃)CH₂CH₂—; and a fluorinated pentamethylene group [—CHFCH₂CH₂CH₂CH₂—, and —CF₂CH₂CH₂CH₂CH₂—].

As the alkylene group having 1 to 5 carbon atoms in V¹⁰¹, an alkylene group having 1 to 4 carbon atoms is preferred, and an alkylene group having 1 to 3 carbon atoms is more preferred.

Specific examples thereof include am methylene group [—CH₂—]; an alkylmethylene group such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; an ethylene group [—CH₂CH₂—], an alkylethylene group such as —CH(CH₃)CH₂—, —CH(CH₃)CH(CH₃)—, —C(CH₃)₂CH₂—, and —CH(CH₂CH₃)CH₂; a trimethylene group (an n-propylene group) [—CH₂CH₂CH₂—]; an alkyltrimethylene group such as —CH(CH₃)CH₂CH₂—, and —CH₂CH(CH₃)CH₂—, a tetramethylene group [—CH₂CH₂CH₂CH₂—]; an alkyl tetramethylene group such as —CH(CH₃)CH₂CH₂CH₂—, and —CH₂CH(CH₃)CH₂CH₂—; and a pentamethylene group [—CH₂CH₂CH₂CH₂CH₂—].

As the fluorinated alkylene group having 1 to 5 carbon atoms in V¹⁰¹, a fluorinated alkylene group having 1 to 4 carbon atoms is preferred, and a fluorinated alkylene group having 1 to 3 carbon atoms is more preferred. A linear fluorinated alkylene group having 1 to 5 carbon atoms is preferred.

Examples of the fluorinated alkylene group having 1 to 5 carbon atoms in V¹⁰¹ include a group in which a part or all of hydrogen atoms in the alkylene group having 1 to 5 carbon atoms in V¹⁰¹ are replaced by fluorine atoms, and in particular, a fluorinated alkylene group in which a carbon atom bonded to the carbon atom to which the adjacent Rf₀₁ and Rf₀₂ are bonded is fluorinated is preferred.

Specific examples of the fluorinated alkylene group having 1 to 5 carbon atoms in V¹⁰¹ include a fluorinated methylene group [—CHF— and —CF₂—]; a fluorinated alkylmethylene group such as —C(CF₃)₂—, —C(CF₃)(CH₂CH₃)—, and —C(CF₃)(CH₂CH₂CH₃)—; a fluorinated ethylene group [—CHFCH₂—, and —CF₂CH₂—]; a fluorinated alkylethylene group such as —CH(CF₃)CH₂—, —CH(CF₃)CH(CH₃)—, and —C(CF₃)₂CH₂—, a fluorinated trimethylene group (a fluorinated n-propylene fluorinated alkyltrimethylene group such as —CH(CF₃)CH₂CH₂— and —CHFCH(CH₃)CH₂; a fluorinated tetramethylene group [—CHFCH₂CH₂CH₂— and —CF₂CH₂CH₂CH₂—]; a fluorinated alkyltetramethylene group such as —CH(CF₃)CH₂CH₂CH₂—, and —CHFCH(CH₃)CH₂CH₂—; and a fluorinated pentamethylene group [—CHFCH₂CH₂CH₂CH₂—, and —CF₂CH₂CH₂CH₂CH₂—].

As the alkylene group having 2 to 30 carbon atoms in V¹⁰², an alkylene group having 2 to 10 carbon atoms is preferred, and an alkylene group having 2 to 5 carbon atoms is more preferred. Specifically, the alkylene group having 2 to 5 carbon atoms as described in V¹⁰¹ is more preferred, and the ethylene group [—CH₂CH₂-] is still more preferred.

As the fluorinated alkylene group having 2 to 30 carbon atoms in V¹⁰², a fluorinated alkylene group having 2 to 10 carbon atoms is preferred, and a fluorinated alkylene group having 2 to 5 carbon atoms is more preferred. Specifically, a fluorinated alkylene group having 2 to 5 carbon atoms as described in V¹⁰¹ are more preferred.

In the general formula (b1-1), X is preferably an alkylene group or a fluorinated alkylene group having a total of 4 or more atoms in the main chain and containing an ester bond. As X, among the linking groups each represented by the above formulae (y-b1-1) to (y-b1-12), any one of the linking groups represented by the above formulae (y-b1-7) and (y-b1-9) is more preferred.

In the general formula (b1-1), Y represents a divalent polycyclic hydrocarbon group that may include a substituent. Examples of the polycyclic hydrocarbon group represented by Y include a group in which two hydrogen atoms are removed from a bicycloalkane, tricycloalkane, or tetracycloalkane having 7 to 30 carbon atoms. Specific examples thereof include a group in which two hydrogen atoms are removed from a polycycloalkane such as adamantane, norbornane, isobornane, tricyclodecane, or tetracyclododecane.

Among the polycyclic group, a group in which two hydrogen atoms are removed from adamantane, a group in which two hydrogen atoms are removed from norbornane, and a group in which two hydrogen atoms are removed from tetracyclododecane are preferred, and a group in which two hydrogen atoms are removed from adamantane and a group in which two hydrogen atoms are removed from norbornane are more preferred. That is, the polycyclic hydrocarbon group represented by Y in the general formula (b1-1) preferably has an adamantane skeleton or a norbomane skeleton.

The polycyclic hydrocarbon group may have a linear or branched alkyl group having 1 to 5 carbon atoms as a substituent.

As the linear or branched alkyl group having 1 to 5 carbon atoms as the substituent, a linear or branched alkyl group having 1 to 4 carbon atoms is preferred, and a methyl group, an ethyl group, a propyl group, an n-butyl group, or a tert-butyl group is more preferred.

In the general formula (b1-1), L represents a divalent linking group containing an ester bond or an oxygen atom, and a plurality of ester bonds and a plurality of oxygen atoms may be each bonded independently via an alkylene group.

Examples of the ester bond include —O—C(═O)— and —C(═O)O.

Preferred examples of the divalent linking group represented by L include —O—, —C(═O)—O—, —C(═O)—, —CH₂—C(═O)—O—, and —CH₂—O—C(═O)—.

Among them, from the viewpoint of raw materials, —O—, —C(═O)—O—, —CH₂—C(═O)—O—, and —CH₂—O—C(═O)— are preferred, and —C(═O)—O—, —CH₂—C(═O)—O—, and —CH₂—O—C(═O)— are particularly preferred.

L is preferably *—O—C(═O)—**, *—CH₂—C(═O)—O—**, or *—CH₂—O—C(═O)—**, and more preferably *—O—C(═O)—** The symbol * represents a bond to be bonded to Y, and ** represents a bond to be bonded to Z.

In the general formula (b1-1), Z represents a substituent represented by the following formula (bz-1).

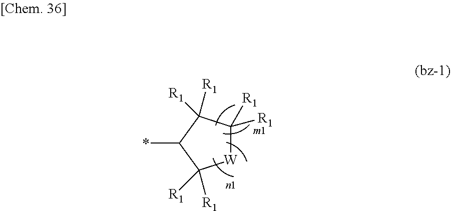

[In the formula (bz-1), R₁'s each independently represent a hydrogen atom, a halogen atom, a linear or branched alkyl group in which a part of carbon atoms may be replaced by a heteroatom, a linear or branched acyl group in which a part of carbon atoms may be replaced by a heteroatom, or a linear or branched alkyloxycarbonyl group in which a part of carbon atoms may be replaced by a heteroatom, W represents an oxygen atom, a sulfur atom, or —NRⁿ—, Rⁿ represents a hydrogen atom, or a linear or branched alkyl group, m1 and n1 each represent an integer of 0 to 4, and m1+n1 represents an integer of 0 to 4, and * represents a bond.]

Examples of the halogen atom in R₁ include a fluorine atom, a chlorine atom, a bromine atom, and an iodine atom, and a fluorine atom is preferred.

Examples of the heteroatom in R₁ include an oxygen atom, a sulfur atom, and a nitrogen atom.

As the linear or branched alkyl group in which a part of carbon atoms are replaced by heteroatoms, a linear or branched alkoxy group, a linear or branched alkoxy alkyl group, a chain or branched alkylthio group, a chain or branched alkylthioalkyl group, a chain or branched alkylamino group, or a chain or branched alkylaminoalkyl group is preferred, a linear or branched alkoxy group, a chain or branched alkylthio group, or a chain or branched alkylamino group is more preferred, and a chain or branched alkoxy group is more preferred. Among the chain or branched alkoxy group, a methoxy group, an ethoxy group, an n-proxy group, an iso-proxy group, an n-butoxy group, and a tert-butoxy group are preferred, a methoxy group and an ethoxy group are more preferred, and a methoxy group is still more preferred.

The linear alkyl group represented by R₁ preferably has 1 to 20 carbon atoms, more preferably has 2 to 15 carbon atoms, still more preferably has 3 to 12 carbon atoms, and particularly preferably has 3 to 10 carbon atoms. Specific examples thereof include a methyl group, an ethyl group, a propyl group, a butyl group, a pentyl group, a hexyl group, a heptyl group, an octyl group, a nonyl group, a decyl group, an undecyl group, a dodecyl group, a tridecyl group, an isotridecyl group, a tetradecyl group, a pentadecyl group, a hexadecyl group, an isohexadecyl group, a heptadecyl group, an octadecyl group, a nonadecyl group, an icosyl group, a heneicosyl group, and a docosyl group.

The branched alkyl group represented by R₁ preferably has 3 to 20 carbon atoms, more preferably has 3 to 15 carbon atoms, and particularly preferably has 3 to 10 carbon atoms. Specific examples thereof include an iso-propyl group, an iso-butyl group, a tert-butyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

The linear acyl group represented by R; is preferably a linear acyl group having 1 to 3 carbon atoms, and specific examples thereof include a formyl group, an acetyl group, a propionyl group, and an acryloyl group.

The branched acyl group represented by R₁ is preferably a branched acyl group having 3 to 10 carbon atoms, and examples thereof include an isobutyryl group and a methacryloyl group.

Examples of the linear or branched alkyloxycarbonyl group represented by R₁ include a linear or branched alkoxycarbonyl group in which a linear or branched alkoxy group having 1 to 4 carbon atoms is bonded to a carbonyl group (specifically, an alkyloxycarbonyl group such as a methoxycarbonyl group, an ethoxycarbonyl group, an iso-propyloxycarbonyl group, or an n-propoxycarbonyl group; an alkenyloxycarbonyl group such as a vinyloxycarbonyl group or an allyloxy carbonyl group).

In the formula (bz-1), it is preferable that R₁'s each independently represent a hydrogen atom or a linear or branched alkyl group in which a part of carbon atoms may be replaced by heteroatoms, it is more preferable that one R₁ represent a linear or branched alkyl group in which a carbon atom may be replaced by a heteroatom, and the other R₁'s represent a hydrogen atom, and it is still more preferable that one R₁ represent a linear or branched alkoxy group or a linear or branched alkyl group, and the other R₁'s represent a hydrogen atom.

Among the linear or branched alkoxy group or linear or branched alkyl group represented by one R₁, a methoxy group, an ethoxy group, an isopropyl group, or a tert-butyl group is more preferred, and a methoxy group or a tert-butyl group is still more preferred.

W represents an oxygen atom, a sulfur atom, or —NRⁿ—, and Rⁿ represents a hydrogen atom or a linear or branched alkyl group.

Examples of the linear alkyl group represented by Rⁿ in —NRⁿ— include a linear alkyl group having 1 to 10 carbon atoms (specifically, a methyl group, an ethyl group, and an n-propyl group).

Examples of the branched alkyl group represented by Rⁿ in —NRⁿ— include a branched alkyl group having 3 to 10 carbon atoms (specifically, an iso-propyl group, and a tert-butyl group).

The symbol m1 preferably represents an integer of 1 to 3, and more preferably 2 or 3.

The symbol n1 preferably represents an integer of 0 to 1, and more preferably represents 1.

In the embodiment of the present invention, it is preferable that, in the formula (bz-1), n1 represent 0, and m1 represent an integer of 2 or 3.

In the embodiment of the present invention, the compound represented by the general formula (b1-1) is preferably a compound represented by the following general formula (b1-2) or general formula (b1-3).

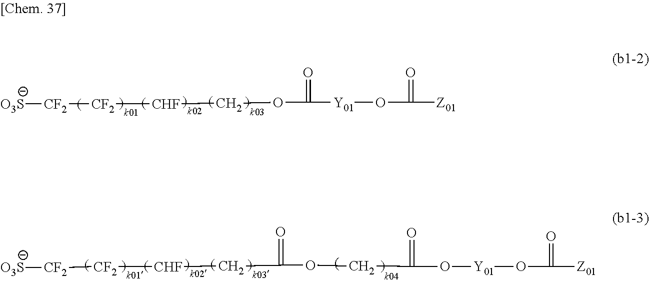

[In the general formula (b1-2) and the general formula (b1-3), k01 and k02 each independently represent an integer of 0 to 2, k01+k02 represents 1 or 2, k01′ and k02′ each independently represent an integer of 0 to 2, k01′+k02′ represents 0 or 1, k03 represents an integer of 1 to 4, k03′ represents an integer of 0 to 4, k04 represents an integer of 1 to 4, Y₀₁ represents a group in which two hydrogen atoms are removed from adamantane or a group in which two hydrogen atoms are removed from norbornane, and Z₀₁ represents a cyclopentyl group or cyclohexyl group which may be replaced by a substituent selected from a halogen atom, a linear or branched alkyl group, a linear or branched acyl group, or an oxycarbonyl group, in which a part of carbon atoms may be replaced by heteroatoms.]

The symbols k01 and k02 each independently preferably represent 0 or 1, and k01+k02 preferably represents 1.

The symbols k01′, k02′ and k03′ each preferably represent 0.

The symbols k03 and k04 each independently preferably represent an integer of 2 or 3, and more preferably represent 2.

Examples of the halogen atom, the linear or branched alkyl group, the linear or branched acyl group, or the oxycarbonyl group, in which a part of carbon atoms may be replaced by heteroatoms in Z₀₁ include the same as those in Z, and preferred examples thereof are also the same.

The substituent of the cyclopentyl group or cyclohexyl group in Z₀₁ is preferably a linear or branched alkoxy group or a linear or branched alkyl group, more preferably a methoxy group, an ethoxy group, an isopropyl group, or a tert-butyl group, and still more preferably a methoxy group or a tert-butyl group.

Specific examples of the anion moiety in the component (B1) are shown below. In the formulae, k represents an integer of 1 to 3. The anion moiety in the component (B1) is not limited to these specific examples.

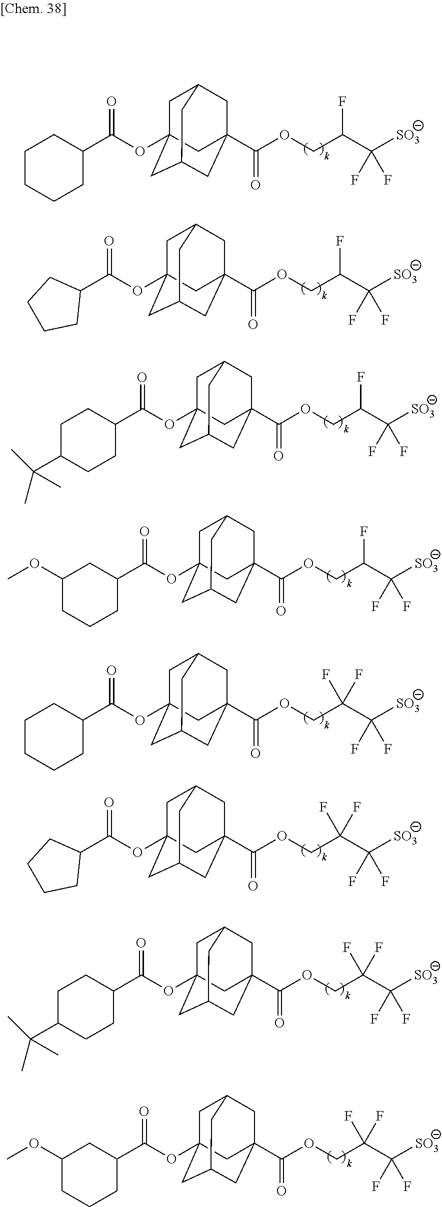

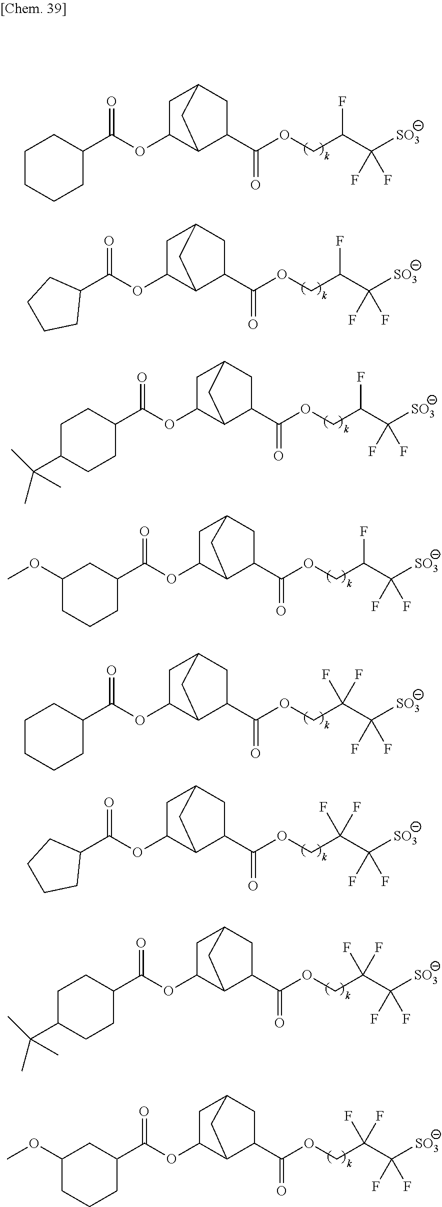

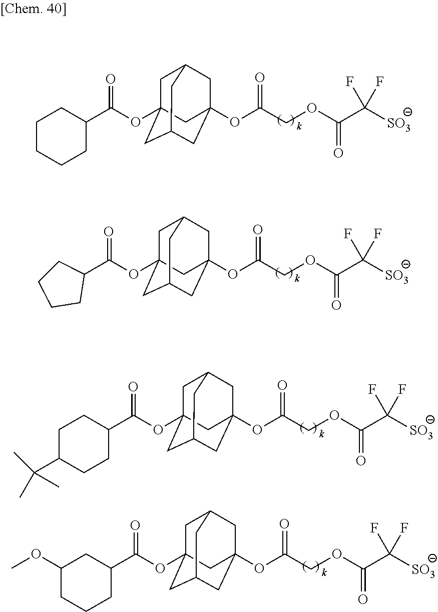

[Cation Moiety: (M^{m+})_{1/m}]

In the general formula (b1-1), M^{m+} represents an m-valent organic cation.

The organic cation in M^{m+} is preferably an onium cation, more preferably a sulfonium cation, an iodonium cation, or an ammonium cation, and still more preferably a sulfonium cation or an iodonium cation. The symbol m is an integer of 1 or more.

Preferred examples of the cation moiety ((M^{m+})_{1/m}) include an organic cation represented by any one of the following formulae (ca-1) to (ca-3).

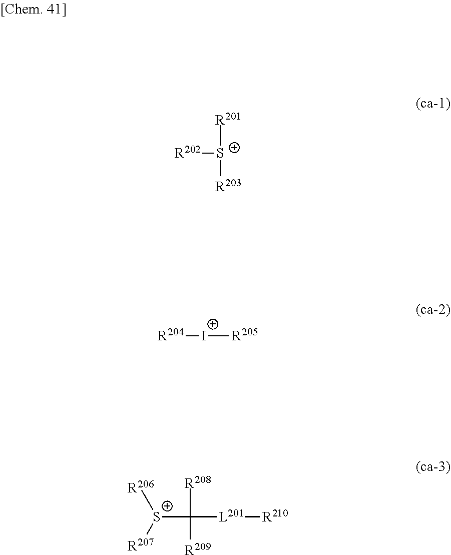

[In the formulae (ca-1) to (ca-3), R²⁰¹ to R²⁰⁷ each independently represent an aryl group, an alkyl group, or an alkenyl group that may include a substituent, and R²⁰¹ to R²⁰³, R²⁰⁶ and R²⁰⁷ may be bonded to each other to form a ring together with the sulfur atom in the formula. R²⁰⁸ and R²⁰⁹ each independently represent a hydrogen atom or an alkyl group having 1 to 5 carbon atoms, R²¹⁰ represents an aryl group that may include a substituent, an alkyl group that may include a substituent, an alkenyl group that may include a substituent, or a —SO₂— containing cyclic group that may include a substituent, and L²⁰¹ represents —C(═O)— or —C(═O)—O—.]

Examples of the aryl group in R²⁰¹ to R²⁰⁷ include an aryl group having 6 to 20 carbon atoms, and a phenyl group or a naphthyl group is preferred.

The alkyl group in R²⁰¹ to R²⁰⁷ is preferably a chain or cyclic alkyl group having 1 to 30 carbon atoms.

The alkenyl group in R²⁰¹ to R²⁰⁷ preferably has 2 to 10 carbon atoms.

Examples of the substituent that R²⁰¹ to R²⁰⁷ may have include an alkyl group, a halogen atom, a halogenated alkyl group, a carbonyl group, a cyano group, an amino group, an aryl group, an arylthio group, and a group represented by any one of the following formulae (ca-r-1) to (ca-r-7).

Examples of the aryl group in the arylthio group as the substituent include an aryl group having 6 to 20 carbon atoms, and a phenyl group, a naphthyl group, and a biphenyl group are preferred. Examples of the arylthio group include a phenylthio group, a naphthylthio group, and a biphenylthio group.

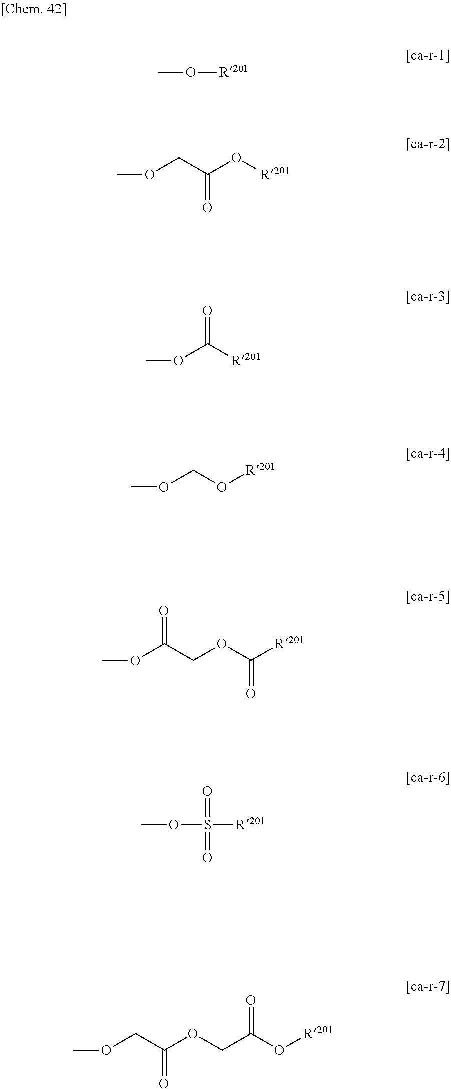

[In the formula, R′²⁰¹'s each independently represent a hydrogen atom, a cyclic group that may include a substituent, a chain alkyl group, or a chain alkenyl group.]

The cyclic group that may include a substituent and is represented by R′²⁰¹ is preferably a cyclic hydrocarbon group, and the cyclic hydrocarbon group may be an aromatic hydrocarbon group or an aliphatic hydrocarbon group.

Examples of the aromatic hydrocarbon group include an aryl group in which one hydrogen atom is removed from an aromatic hydrocarbon ring or an aromatic compound containing two or more aromatic rings, and a phenyl group and a naphthyl group are preferred.

Examples of the aliphatic hydrocarbon group include a group in which one hydrogen atom is removed from a monocycloalkane or a polycycloalkane, and an adamantyl group and a norbornyl group are preferred.

The chain alkyl group that may include a substituent and is represented by R′²⁰¹ may be either linear or branched.

The linear alkyl group preferably has 1 to 20 carbon atoms, more preferably has 1 to 15 carbon atoms, and most preferably has 1 to 10 carbon atoms. Specific examples thereof include a methyl group, an ethyl group, a propyl group, a butyl group, a pentyl group, a hexyl group, a heptyl group, an octyl group, a nonyl group, a decyl group, an undecyl group, a dodecyl group, a tridecyl group, an isotridecyl group, a tetradecyl group, a pentadecyl group, a hexadecyl group, an isohexadecyl group, a heptadecyl group, an octadecyl group, a nonadecyl group, an icosyl group, a heneicosyl group, and a docosyl group.

The chain alkenyl group that may include a substituent and is represented by R′²⁰¹ may be either linear or branched, and preferably has 2 to 10 carbon atoms, more preferably has 2 to 5 carbon atoms, still more preferably has 2 to 4 carbon atoms, and particularly preferably has 3 carbon atoms. Examples of the linear alkenyl group include a vinyl group, a propenyl group (an allyl group), and a butenyl group. Examples of the branched alkenyl group include a 1-methylpropenyl group and a 2-methylpropenyl group.

Among them, the chain alkenyl group is particularly preferably a propenyl group.

Examples of the cyclic group that may include a substituent and is represented by R′²⁰¹ or the chain alkyl group that may include a substituent and is represented by R′²⁰¹ include those same as the acid-dissociable group represented by the formula (a1-r-2).

Examples of the substituent in the cyclic group, the chained alkyl group, or the chain alkenyl group which are represented by R′²⁰¹ include an alkyl group, an alkoxy group, a halogen atom, a halogenated alkyl group, a hydroxy group, a carbonyl group, and a nitro group.

The alkyl group as the substituent is preferably an alkyl group having 1 to 5 carbon atoms, and most preferably a methyl group, an ethyl group, a propyl group, an n-butyl group, or a tert-butyl group.

The alkoxy group as the substituent is preferably an alkoxy group having 1 to 5 carbon atoms, more preferably a methoxy group, an ethoxy group, an n-propoxy group, an iso-propoxy group, an n-butoxy group, or a tert-butoxy group, and most preferably a methoxy group or an ethoxy group.

Examples of the halogen atom as the substituent include a fluorine atom, a chlorine atom, a bromine atom, and an iodine atom. A fluorine atom is preferred.

Examples of the halogenated alkyl group as the substituent include an alkyl group having 1 to 5 carbon atoms, for example, a group in which a part or all of hydrogen atoms of a methyl group, an ethyl group, a propyl group, an n-butyl group, a tert-butyl group, or the like are replaced by halogen atoms.

When R²⁰¹ to R²⁰³ and R²⁰⁶ and R²⁰⁷ are bonded to each other to form a ring together with the sulfur atom in the formula. R²⁰¹ to R²⁰³ and R²⁰⁶ and R²⁰⁷ may be bonded via a heteroatom such as a sulfur atom, an oxygen atom or a nitrogen atom, or a functional group such as a carbonyl group, —SO—, —SO₂—, —SO₃—, —COO—, —CONH— or —N(R_{N})— (the R_{N} is an alkyl group having 1 to 5 carbon atoms). Regarding the ring to be formed, one ring containing the sulfur atom of the formula in the ring skeleton is preferably a 3-membered to 10-membered ring, and particularly preferably a 5-membered to 7-membered ring, including the sulfur atom. Specific examples of the ring to be formed include a thiophene ring, a thiazole ring, a benzothiopbene ring, a thianthlene ring, a benzothiophene ring, a dibenzthiophene ring, a 9H-thioxanthene ring, a thioxanthone ring, a thianthrene ring, a phenoxathiin ring, a tetrahydrothiophenium ring, a tetrahydrothiopyranium ring, and a thioxanium ring.

R²⁰⁸ and R²⁰⁹ each independently represent a hydrogen atom or an alkyl group having 1 to 5 carbon atoms, and are preferably a hydrogen atom or an alkyl group having 1 to 3 carbon atoms. When R²⁰² and R²⁰⁹ represent an alkyl group, R²⁰⁸ and R²⁰⁹ may be bonded to each other to form a ring.

R²¹⁰ represents an aryl group that may include a substituent, an alkyl group that may include a substituent, an alkenyl group that may include a substituent, or a —SO₂— containing cyclic group that may include a substituent.

Examples of the aryl group in R²¹⁰ include an unreplaced aryl group having 6 to 20 carbon atoms, and a phenyl group and a naphthyl group are preferred.

The alkyl group in R²¹⁰ is preferably a chain or cyclic alkyl group having 1 to 30 carbon atoms.

The alkenyl group in R210 preferably has 2 to 10 carbon atoms.

In the cyclic group containing —SO₂— that may include a substituent in R²¹⁰, the “—SO₂— containing cyclic group” refers to a cyclic group containing a ring containing —SO₂— in the ring skeleton thereof, and specifically, the —SO₂— containing cyclic group is a cyclic group in which the sulfur atom(S) in —SO₂— forms a part of the ring skeleton of the cyclic group. When a ring containing —SO₂— in the ring skeleton thereof is counted as a first ring, a group having only the ring is referred to as a monocyclic group, and a group further having another ring structure is referred to as a polycyclic group regardless of the structure. The —SO₂— containing cyclic group may be a monocyclic group or a polycyclic group.

The —SO₂— containing cyclic group is particularly preferably a cyclic group containing —O—SO₂— in the ring skeleton thereof, that is, a cyclic group containing a sultone ring in which —O—S— in —O—SO₂— forms a part of the ring skeleton.

The —SO₂— containing cyclic group that may include a substituent in R²¹⁰ is preferably a group represented by the formula (a5-1-1).

Examples of the substituent that R²¹⁰ may have include an alkyl group, a halogen atom, a halogenated alkyl group, a carbonyl group, a cyano group, an amino group, an aryl group, and an arylthio group.

In the embodiments of the present invention, among the above formulae (ca-1) to (ca-3), a cation represented by a formula (ca-1) is preferred, and as the cation represented by the formula (ca-1), a cation represented by the following general formula (b-2) is more preferred.

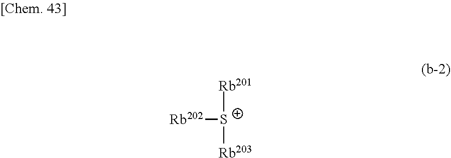

[In the general formula (b-2), Rb²⁰¹ represents an aryl group that may include a substituent. Rb²⁰² and Rb²⁰³ each independently represent an aryl group that may include a substituent, an alkyl group that may include a substituent, or an alkenyl group that may include a substituent. Rb²⁰¹ to Rb²⁰³ may be bonded to each other to form a ring together with a sulfur atom in the general formula (b-2).]

The aryl group that may include a substituent and is represented by any one of Rb²⁰¹ to Rb²⁰³ has the same meaning as the aryl group that may include a substituent as any one of R²⁰¹ to R²⁰⁷, and preferred examples of the aryl group of Rb²⁰¹ to Rb²⁰³ are the same as those of the aryl group of R²⁰¹ to R²⁰⁷.

The alkyl group and alkenyl group that may include a substituent and is represented by Rb²⁰² and Rb²⁰³ have the same meanings as the alkyl group and alkenyl group that may include a substituent and is represented by R²⁰¹ to R²⁰⁷, and preferred examples of the alkyl group and alkenyl group that may include a substituent and is represented by Rb²⁰² and Rb²⁰³ are the same as those of the alkyl group and alkenyl group of R²⁰¹ to R²⁰⁷.

Specific examples of the suitable cation represented by the general formula (ca-1) include cations each represented by the following formulas (ca-1-1) to (ca-1-67).

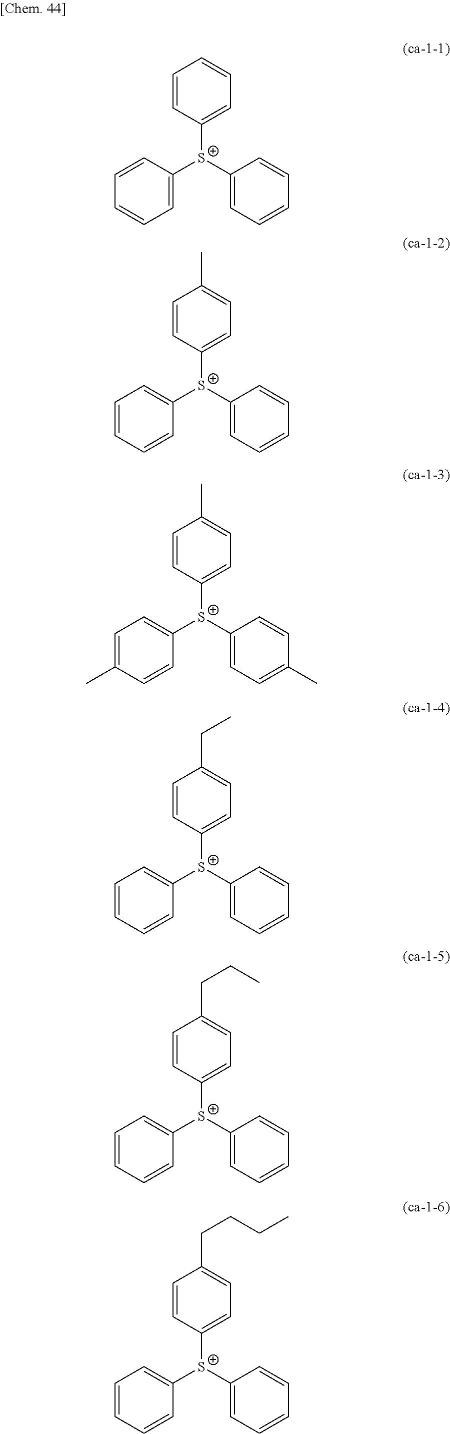

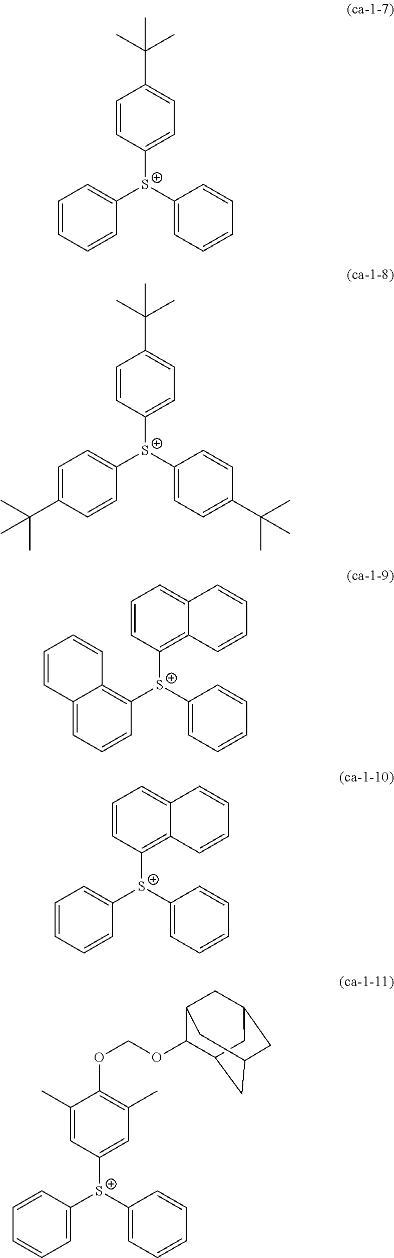

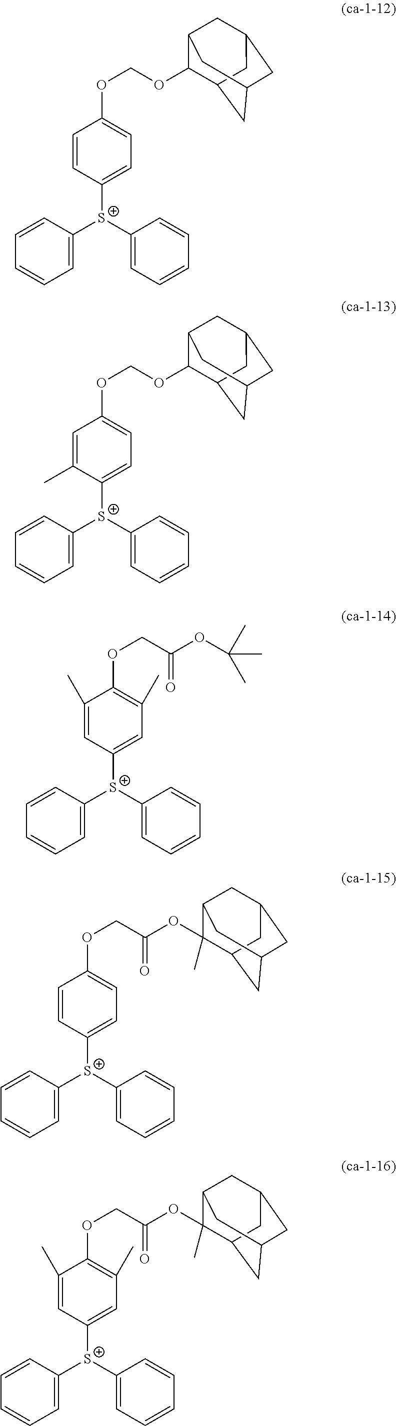

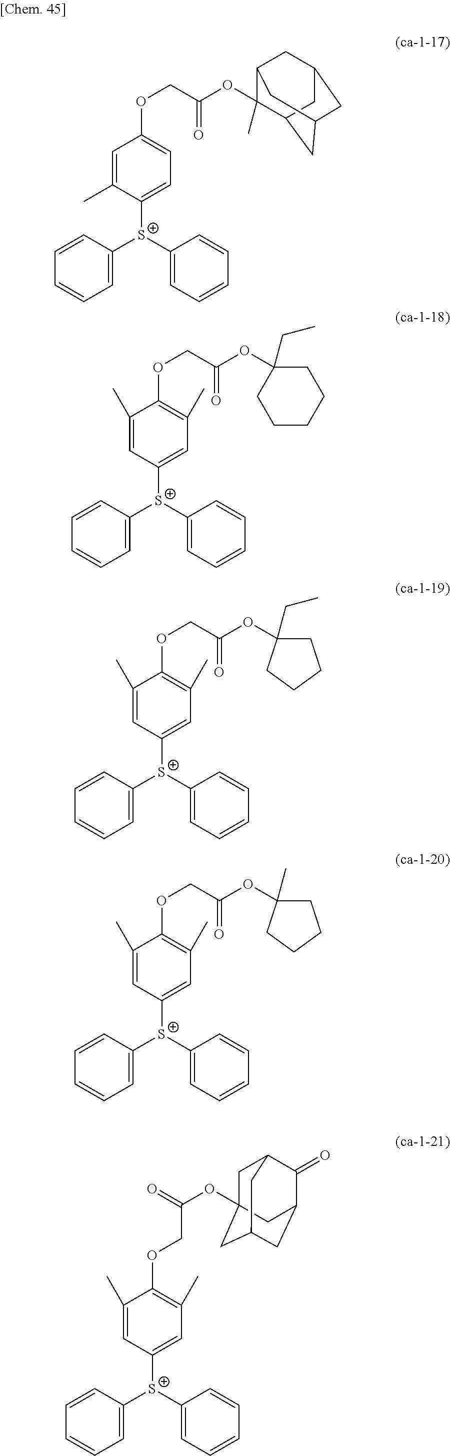

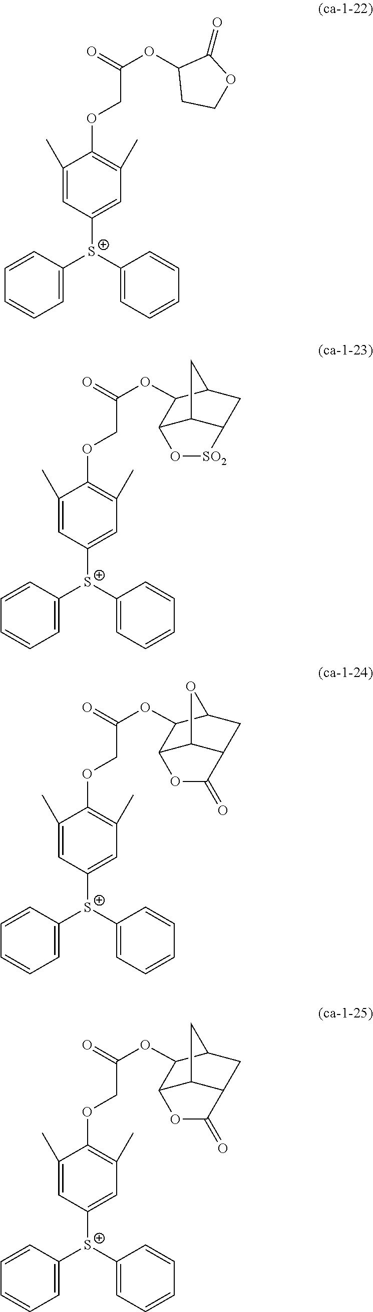

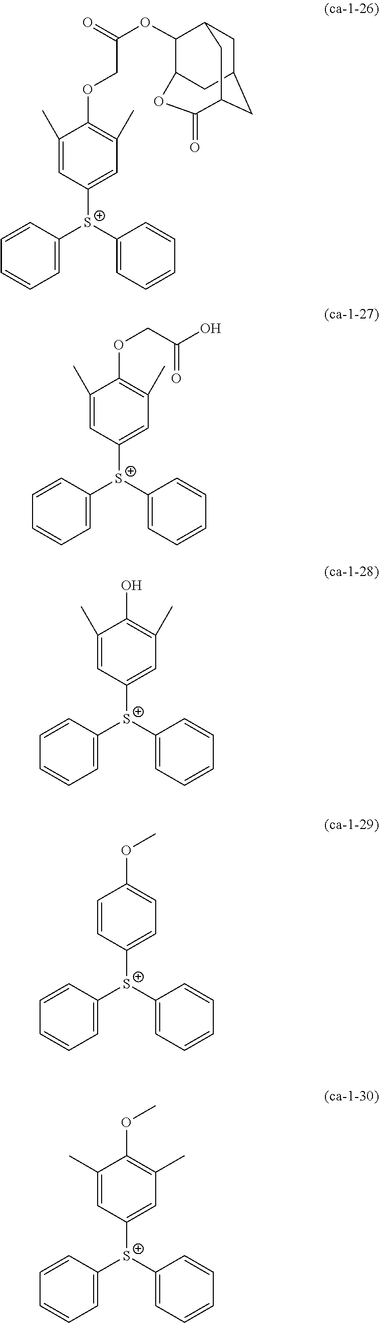

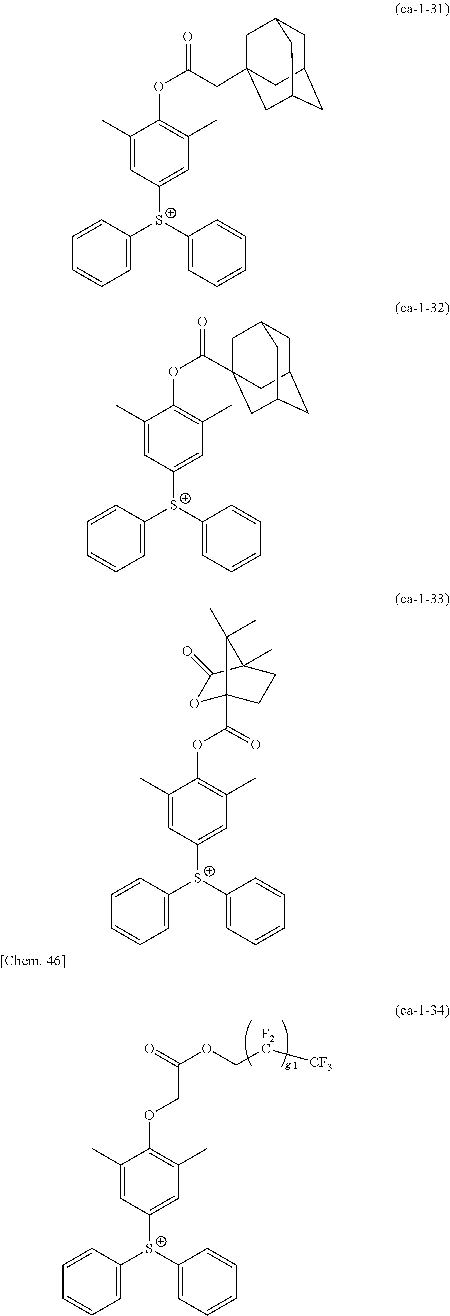

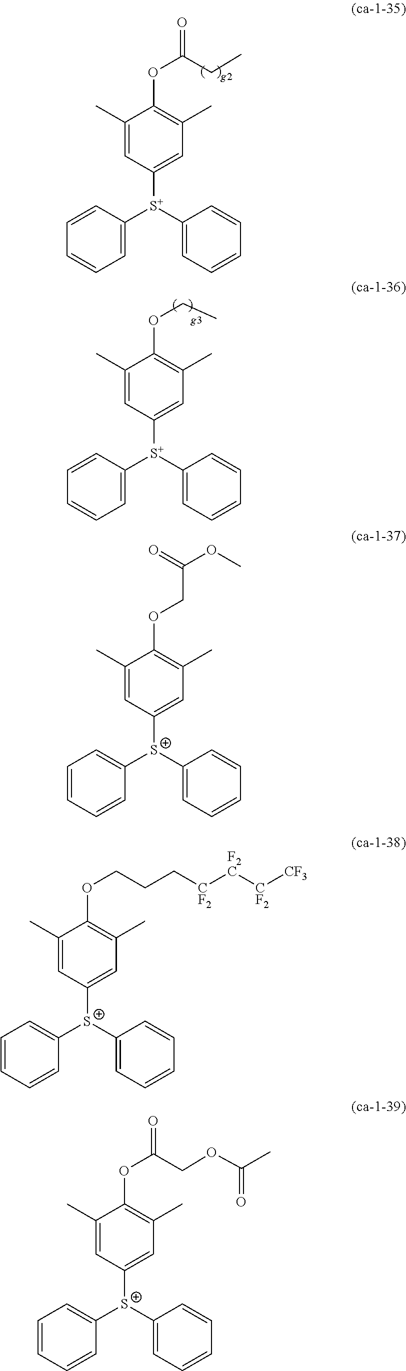

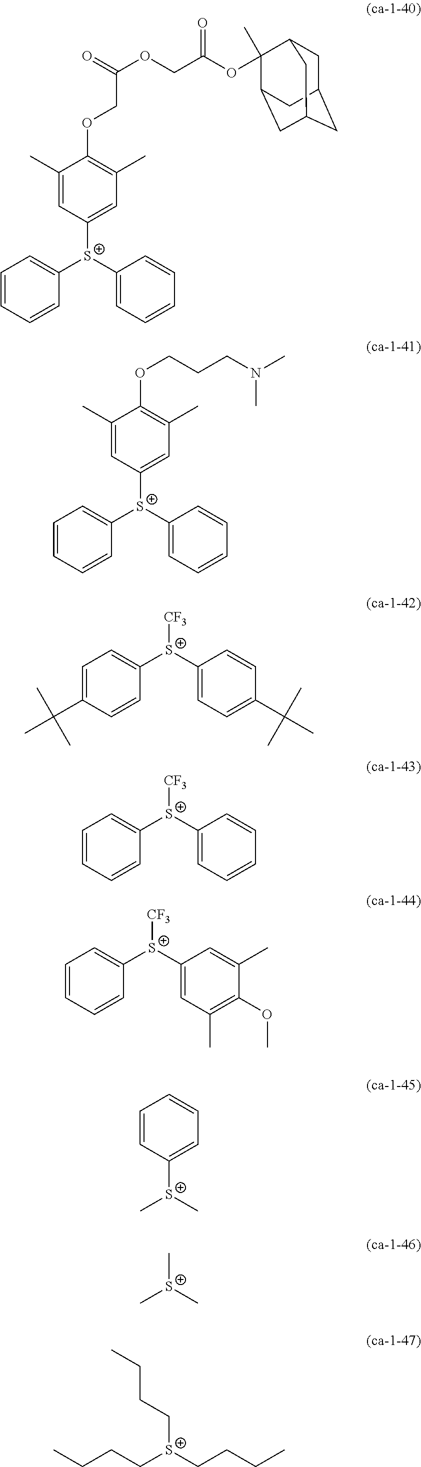

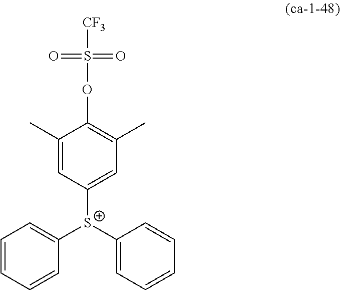

[In the formulae, g1, g2, and g3 represent the number of repetitions, g1 is an integer of 1 to 5, g2 is an integer of 0 to 20, and g3 is an integer of 0 to 20.]

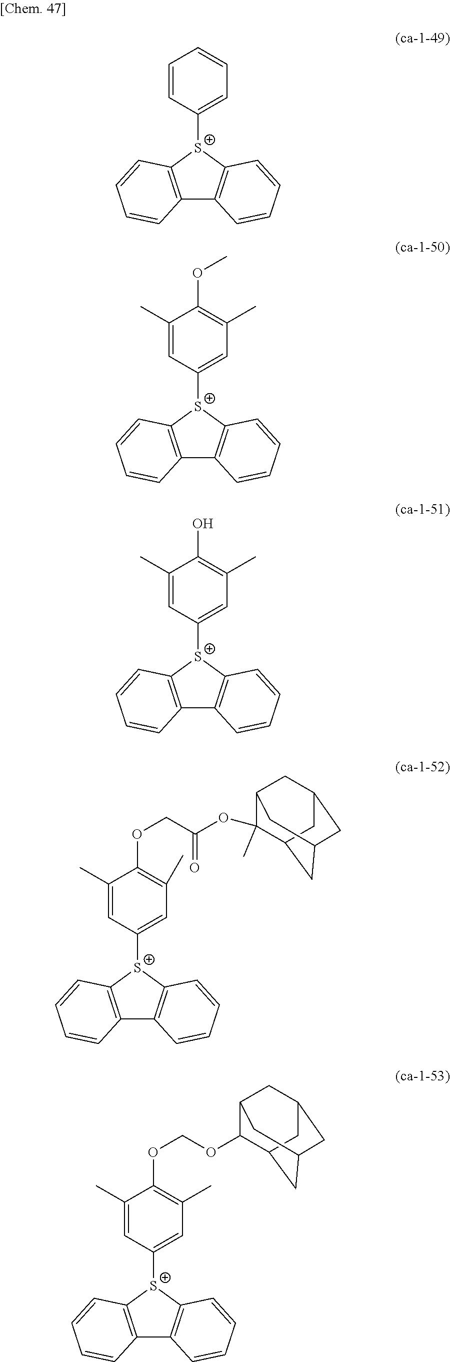 [SMILES: c1ccc(-[s+]2c3ccccc3c3ccccc32)cc1 ; COc1c(C)cc(-[s+]2c3ccccc3c3ccccc32)cc1C ; Cc1cc(-[s+]2c3ccccc3c3ccccc32)cc(C)c1O ; Cc1cc(-[s+]2c3ccccc3c3ccccc32)cc(C)c1OCC(=O)OC1(C)C2CC3CC(C2)CC1C3 ; Cc1cc(-[s+]2c3ccccc3c3ccccc32)cc(C)c1OCOC1C2CC3CC(C2)CC1C3]

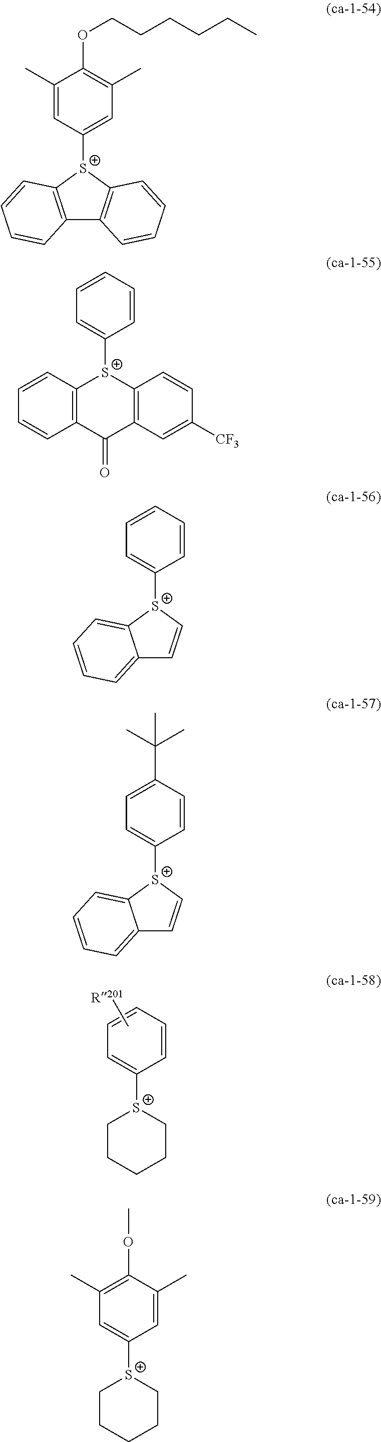

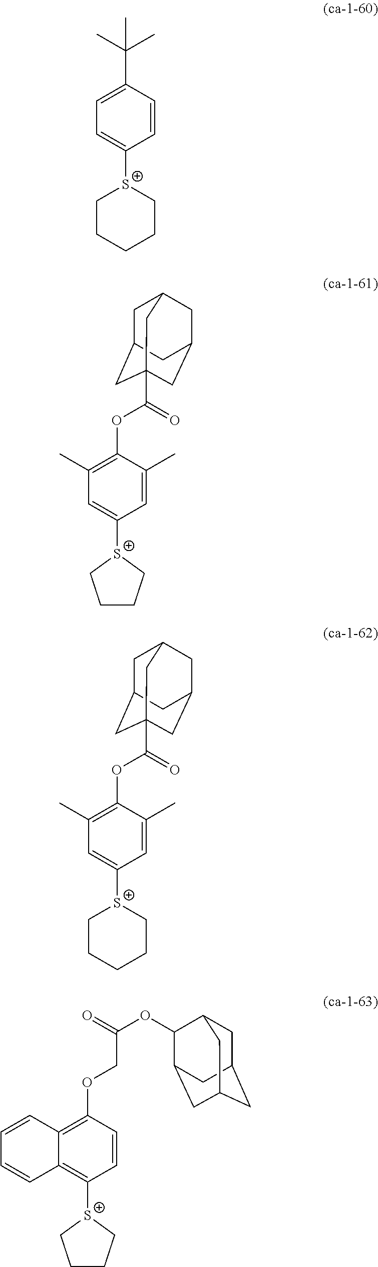 [SMILES: CC(C)(C)c1ccc([S+]2CCCCC2)cc1 ; Cc1cc([S+]2CCCC2)cc(C)c1OC(=O)C12CC3CC(CC(C3)C1)C2 ; Cc1cc([S+]2CCCCC2)cc(C)c1OC(=O)C12CC3CC(CC(C3)C1)C2 ; O=C(COc1ccc([S+]2CCCC2)c2ccccc12)OC1C2CC3CC(C2)CC1C3]

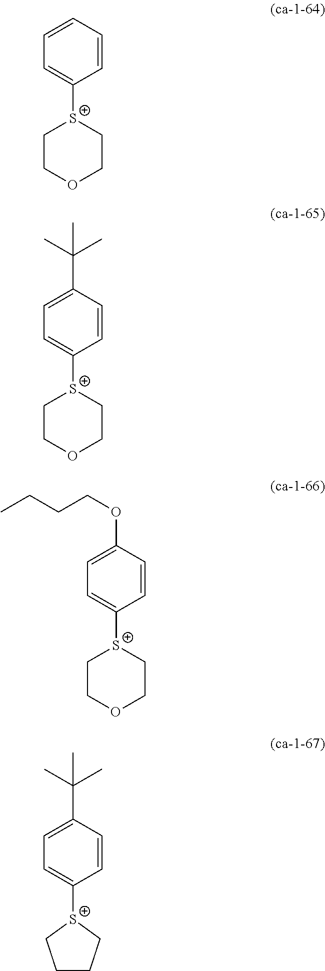 [SMILES: c1ccc([S+]2CCOCC2)cc1 ; CC(C)(C)c1ccc([S+]2CCOCC2)cc1 ; CCCCOc1ccc([S+]2CCOCC2)cc1 ; CC(C)(C)c1ccc([S+]2CCCC2)cc1]

[In the formulae, R″²⁰¹ represents a hydrogen atom or a substituent, and the substituent is the same as the substituent that the R²⁰¹ to R²⁰⁷ may include.]

Specific examples of the suitable cation represented by any one of the formula (ca-3) include a cation represented by any one of the following formulae (ca-3-1) to (ca-3-6).

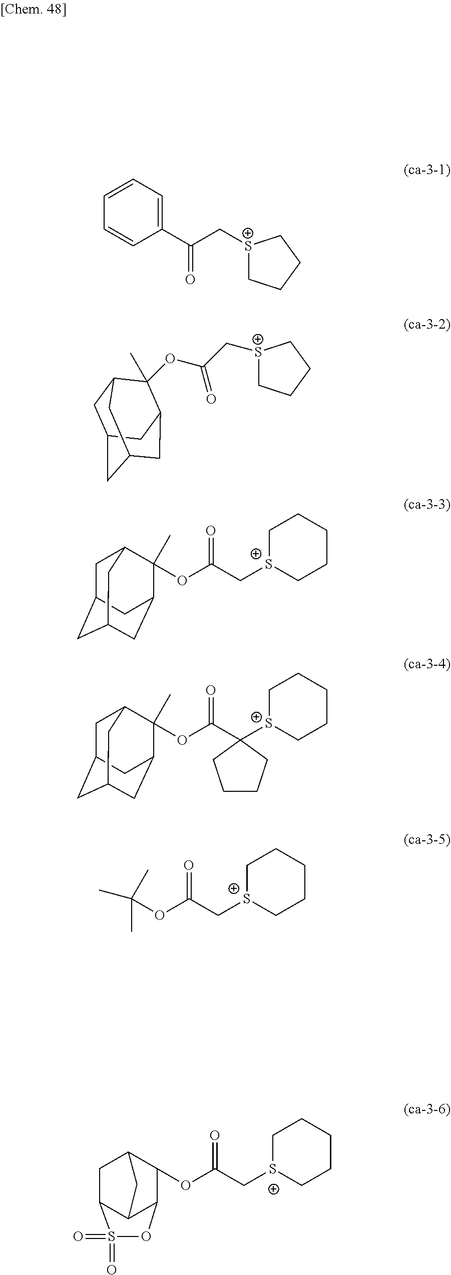 [SMILES: O=C(C[S+]1CCCC1)c1ccccc1 ; CC1(OC(=O)C[S+]2CCCC2)C2CC3CC(C2)CC1C3 ; CC1(OC(=O)C[S+]2CCCCC2)C2CC3CC(C2)CC1C3 ; CC1(OC(=O)C2([S+]3CCCCC3)CCCC2)C2CC3CC(C2)CC1C3 ; CC(C)(C)OC(=O)C[S+]1CCCCC1 ; O=C(C[S+]1CCCCC1)OC1C2CC3C1OS(=O)(=O)C3C2]

In the compound represented by the general formula (b1-1), when the organic cation in M^{m+} is an ammonium cation, examples of the cation include the following.

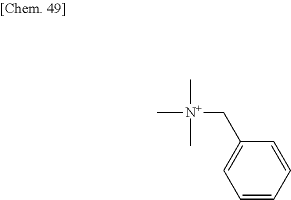

In the compound represented by the general formula (b1-1), a compound in which the organic cation in M^{m+} is an ammonium cation is also useful as a synthetic intermediate for a compound in which the organic cation in M^{m+} is a sulfonium cation or an iodonium cation.

Specific examples of the suitable component (B1) are shown below.

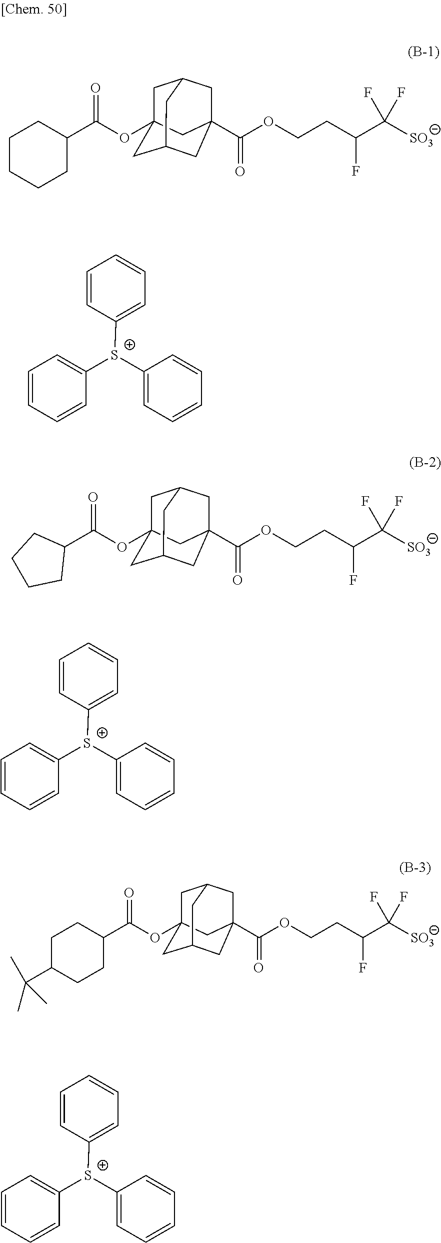

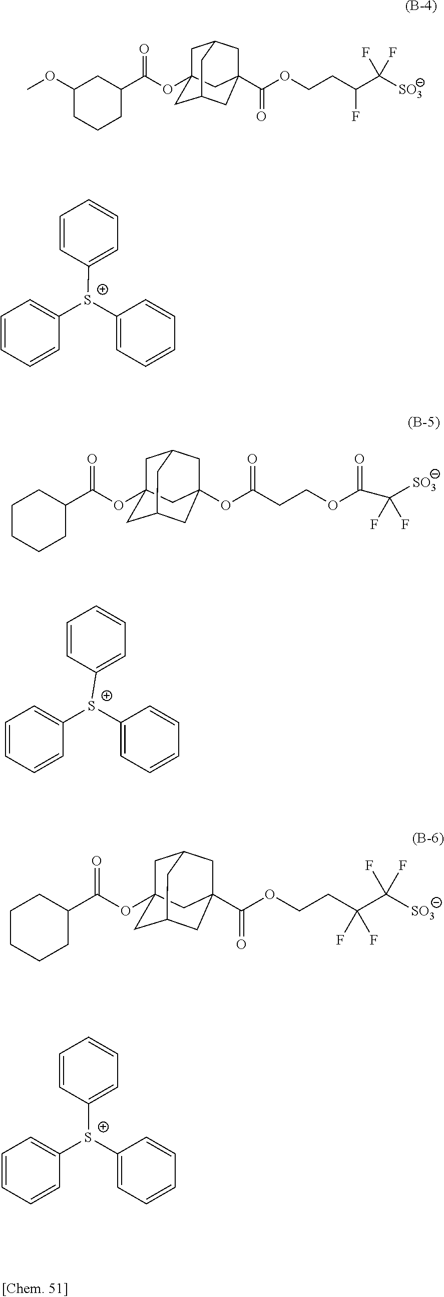

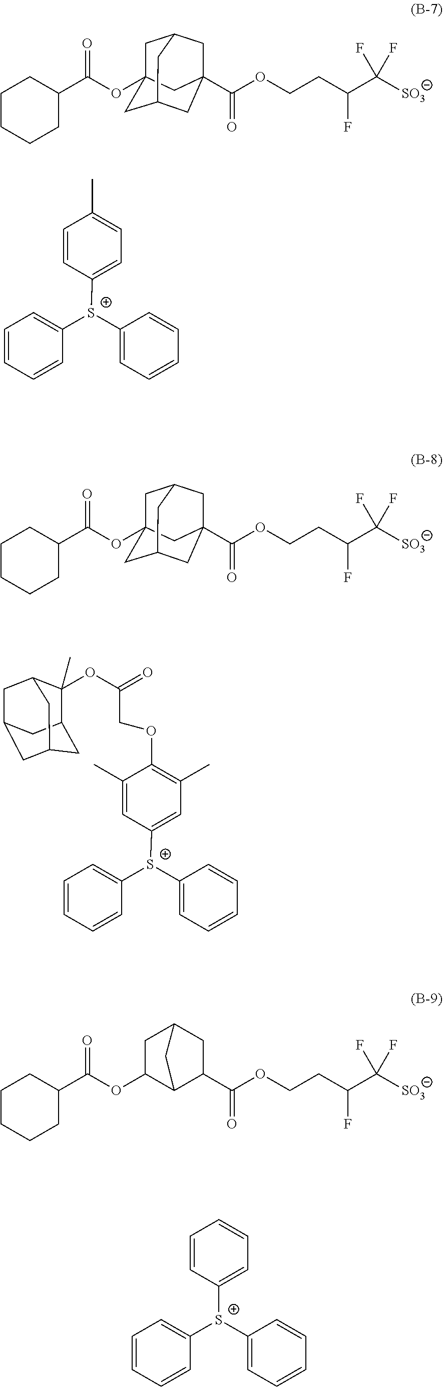

In the resist composition according to the embodiment of the present invention, the component (B1) may be used alone or in combination of two or more kinds thereof.

In the resist composition according to the embodiment of the present invention, a content of the component (B1) is preferably 1 part by mass to 40 parts by mass, more preferably 2.5 parts by mass to 30 parts by mass, and still more preferably 5 parts by mass to 25 parts by mass, with respect to 100 parts by mass of the component (A).

When the proportion of the component (B1) is equal to or larger than the preferred lower limit value, lithography properties such as reduced roughness and a high sensitivity are further improved. On the other hand, when the proportion of the component (B1) is equal to or less than the upper limit value, a resist pattern having excellent exposure latitude becomes easily formed.

### Regarding Component (B2)

The resist composition according to the embodiment of the present invention may contain an acid generating agent component (hereinafter, referred to as “component (B2)”) other than the component (B1) as long as the effects of the present invention are not impaired.

The component (B2) is not particularly limited, and those proposed as an acid generating agent for a chemically amplified resist composition can be used.

Examples of such an acid generating agent include various acid generating agents such as onium salt acid generating agents such as an iodonium salt and a sulfonium salt; oxime sulfonate acid generating agents; diazomethane acid generating agents such as bisalkyl or bisarylsulfonyl diazomethanes and poly(bissulfonyl) diamethanes; nitrobenzyl sulfonate acid generating agents; iminoosulfonate acid generating agents; and disulfone acid generating agents.

In the resist composition according to the embodiment of the present invention, the component (B2) may be used alone or in combination of two or more kinds thereof.

When the resist composition contains the component (B2), a content of the component (B2) in the resist composition is preferably 30 parts by mass or less, and more preferably 1 part by mass to 25 parts by mass, with respect to 100 parts by mass of the component (A).

When the content of the component (B2) in the resist composition is within the above range, pattern formation is sufficiently performed. It is preferable that the content of the component (B2) is within the range since a uniform solution is easily obtained when each component of the resist composition is dissolved in an organic solvent, and storage stability of the resist composition is improved.

In the resist composition according to the embodiment of the present invention, a content of the acid generating agent component (B) is preferably 1 part by mass to 40 parts by mass, more preferably 2.5 parts by mass to 30 parts by mass, and still more preferably 5 parts by mass to 25 parts by mass, with respect to 100 parts by mass of the base material component (A).

By setting the content of acid generating agent component (B) in the resist composition within the above range, a resist composition having excellent stability over time is easily obtained, and a resist pattern having both a high exposure latitude and a high sensitivity to a light source for exposure is easily formed.

### <Optional Component>

The resist composition according to the embodiment of the present invention may further contain a component (an optional component) other than the component (A) and component (B).

Examples of the optional component include the following components (D), (B), (F), and(S).

### <<Component (D)>>

The resist composition according to the present embodiment may further contain a base component (hereinafter, referred to as “component (D)”) in addition to the component (A) and the component (B), The component (D) acts as a quencher (an acid diffusion control agent) that traps acid generated in the resist composition upon exposure.

The component (D) may be a photodegradable base (D1) (hereinafter, referred to as “component (D1)”) which is decomposed upon exposure and loses acid diffusion controllability, or may be a nitrogen-containing organic compound (D2) (hereinafter, referred to as “component (D2)”) not corresponding to the component (D1), and is preferably the component (D1).

By using a resist composition containing the component (D), a contrast between an exposed portion and an unexposed portion of the resist film can be further improved when forming a resist pattern.

### Regarding Component (D1)

By using a resist composition containing the component (D1), a contrast between an exposed portion and an unexposed portion of the resist film can be further improved when forming a resist pattern.

The component (D1) may be used as an acid generating agent in addition to the component (B) or in place of the component (B).

The component (D1) is not particularly limited as long as it is decomposed upon exposure and loses acid diffusion controllability, and is preferably one or more acid diffusion control agents (d) selected from the group consisting of a compound represented by the following general formula (d1-1) (hereinafter, referred to as “component (d1-1)”), a compound represented by the following general formula (d1-2) (hereinafter, referred to as “component (d1-2)”), and a compound represented by the following general formula (d1-3) (hereinafter, referred to as “component (d1-3)”).

The resist composition in the present embodiment preferably further includes the acid diffusion control agent (d) including a compound represented by any one of the following general formulae (d1-1) to (d1-3).

The components (d1-1) to (d1-3) do not act as a quencher in an exposed portion of the resist film because they are decomposed and lose acid diffusion controllability (basicity), but act as a quencher in an unexposed portion of the resist film.

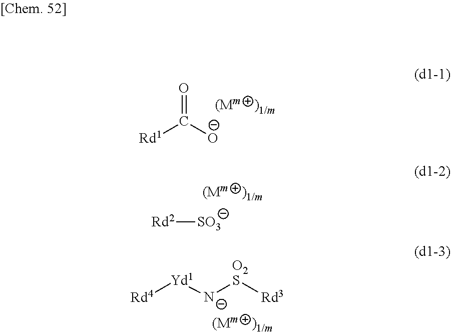

[In the formulae, Rd¹ to Rd⁴ represent a cyclic group that may include a substituent, a chain alkyl group that may include a substituent, or a chain alkenyl group that may include a substituent. In Rd² in the formula (d1-2), no fluorine atom is bonded to a carbon atom adjacent to an S atom, Yd¹ represents a single bond or a divalent linking group, m represents an integer of 1 or more, and M^{m+}'s each independently represent an m-valent organic cation.]

{(Component (d1-1)}

### Anion Moiety

In the formula (d1-1), Rd¹ represents a cyclic group that may include a substituent, a chain alkyl group that may include a substituent, or a chain alkenyl group that may include a substituent, each of which is the same as R²⁰¹ described above.

Among them, as Rd¹, an aromatic hydrocarbon group that may include a substituent, an aliphatic cyclic group that may include a substituent, and a chain alkyl group that may include a substituent are preferred. Examples of the substituent that the groups may have include a hydroxy group, an oxo group, an alkyl group, an aryl group, a fluorine atom, a fluorinated alkyl group, a lactone-containing cyclic group, an ether bond, an ester bond, or a combination thereof. When an ether bond or an ester bond is contained as the substituent, an alkylene group may be interposed, and as the substituent in this case, a linking group represented any one of by the following formulae (y-a1-1) to (y-a1-8) is preferred.

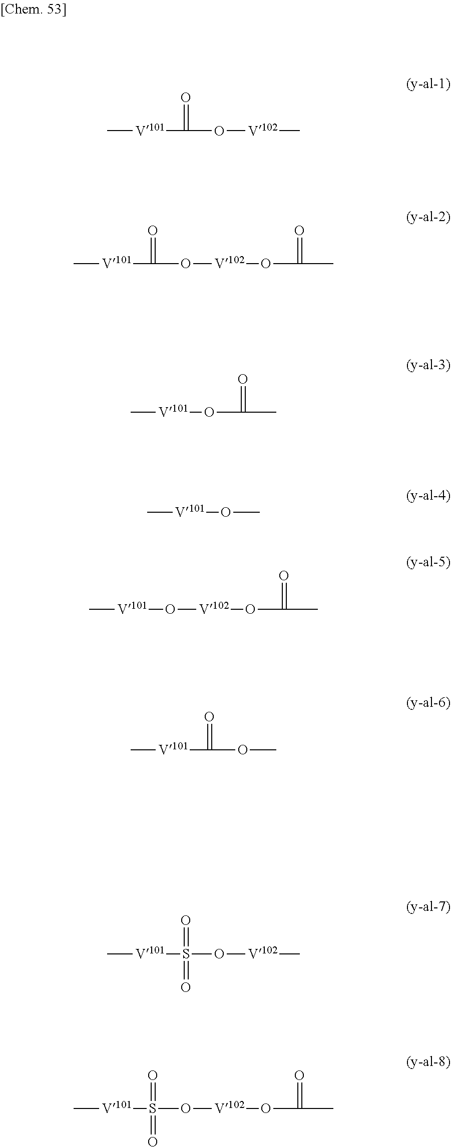

[In the formulae, V′¹⁰¹ of represents a single bond or an alkylene group having 1 to 5 carbon atoms, and V′¹⁰² represents a divalent saturated hydrocarbon group having 1 to 30 carbon atoms.]

In the formulae, the divalent saturated hydrocarbon group in V′¹⁰² is preferably an alkylene group having 1 to 30 carbon atoms. The alkylene group in V′¹⁰² is preferably an alkylene group having 1 to 30 carbon atoms, more preferably an alkylene group having 1 to 10 carbon atoms, and still more preferably an alkylene group having 1 to 5 carbon atoms.

Suitable examples of the aromatic hydrocarbon group include a phenyl group, a naphthyl group, and a polycyclic structure containing a bicyclooctane skeleton (a polycyclic structure consisting of a bicyclooctane skeleton and other ring structures).

The aliphatic cyclic group is more preferably a group in which one or more hydrogen atoms are removed from a polycycloalkane such as adamantane, norbornane, isobornane, tricyclodecane, or tetracyclododecane.

The chain alkyl group preferably has 1 to 10 carbon atoms. Specific examples thereof include a linear alkyl group such as a methyl group, an ethyl group, a propyl group, a butyl group, a pentyl group, a hexyl group, a heptyl group, an octyl group, a nonyl group, and a decyl group; and a branched alkyl group such as a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

When the chain alkyl group is a fluorinated alkyl group having a fluorine atom or a fluorinated alkyl group as a substituent, the number of carbon atoms of the fluorinated alkyl group is preferably 1 to 11, more preferably 1 to 8, and still more preferably 1 to 4. The fluorinated alkyl group may contain an atom other than a fluorine atom. Examples of the atom other than a fluorine atom include an oxygen atom, a sulfur atom, and a nitrogen atom.

Rd¹ is preferably a fluorinated alkyl group in which a part or all of hydrogen atoms constituting a linear alkyl group are replaced by fluorine atoms, and particularly preferably a fluorinated alkyl group in which all of the hydrogen atoms constituting the linear alkyl group are replaced by fluorine atoms (a linear perfluoroalkyl group).

Specific preferred examples of the anion moiety of the component (d1-1) are shown below.

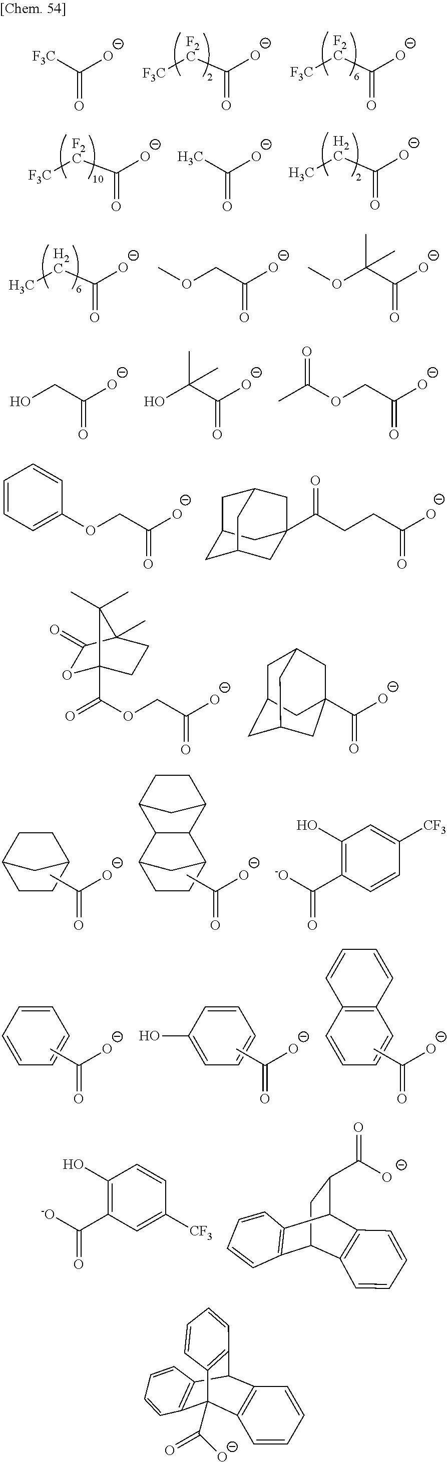

### Cation Moiety

In the formula (d1-1), M^{m+} represents an m-valent organic cation.

Suitable examples of the organic cation of M^{m+} include those same as a cation represented by any one of the general formulae (ca-1) to (ca-3). A cation represented by the general formula (ca-1) is more preferred, and a cation represented by any one of the formulae (ca-1-1) to (ca-1-67) is even more preferred.

The component (d1-1) may be used alone or in combination of two or more kinds thereof.

{Component (d1-2)}

### Anion Moiety

In the formula (d1-2), Rd² represents a cyclic group that may include a substituent, a chain alkyl group that may include a substituent, or a chain alkenyl group that may include a substituent, each of which is the same as R′²⁰¹ described above.

In Rd², a carbon atom adjacent to the S atom does not have a fluorine atom bonded thereto (is not replaced by fluorine). Accordingly, the anion of the component (d1-2) becomes an appropriate weak acid anion, and a quenching ability of the component (D) is improved.

Rd² is preferably a chain alkyl group that may include a substituent, or an aliphatic cyclic group that may include a substituent. The chain alkyl group preferably has 1 to 10 carbon atoms, and more preferably has 3 to 10 carbon atoms. The aliphatic cyclic group is more preferably a group (that may include a substituent) in which one or more hydrogen atoms are removed from adamantane, norbornane, isobornane, tricyclodecane, tetracyclododecane, or the like; or a group in which one or more hydrogen atoms are removed from camphor, or the like.

The hydrocarbon group for Rd² may include a substituent, and examples of the substituent include those same as substituents that the hydrocarbon group (the aromatic hydrocarbon group, the aliphatic cyclic group, or the chained alkyl group) for Rd′ in the formula (d1-1) may have.

Specific preferred examples of the anion moiety of the component (d1-2) are shown below.

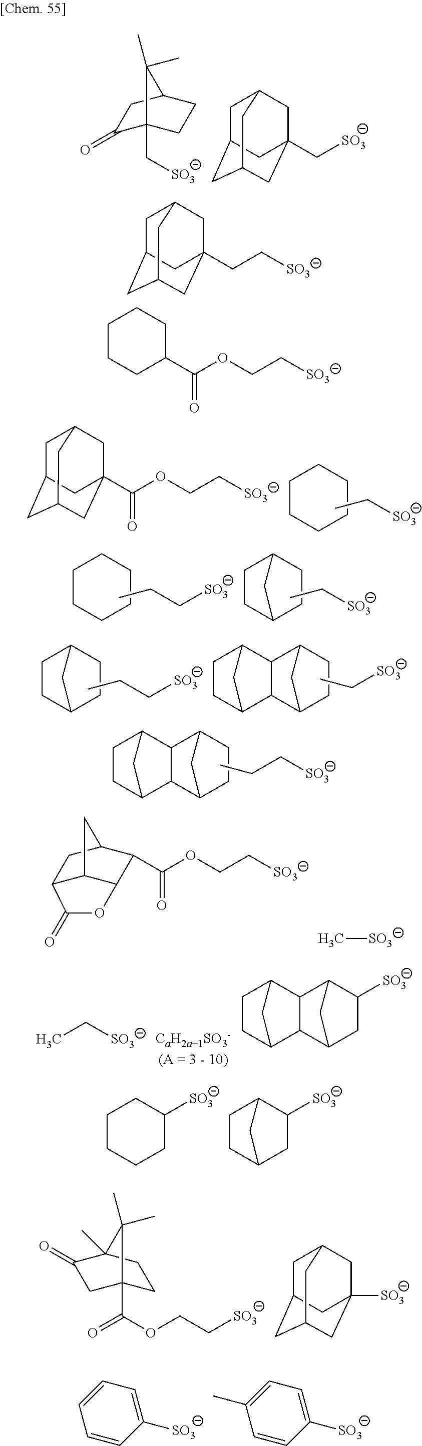

### Cation Moiety

In the formula (d1-2), M^{m+} represents an m-valent organic cation, and is the same as M^{m+} in the formula (d1-1).

The component (d1-2) may be used alone or in combination of two or more kinds thereof.

{Component (d1-3)}

### Anion Moiety

In the formula (d1-3), Rd³ represents a cyclic group that may include a substituent, a chain alkyl group that may include a substituent, or a chain alkenyl group that may include a substituent, each of which is the same as R′²⁰¹ described above, and a fluorine atom-containing cyclic group, a chain alkyl group, or a chain alkenyl group is preferred. Among them, a fluorinated alkyl group is preferred, and a fluorinated alkyl group same as those described above for Rd¹ is more preferred.

In the formula (d1-3), Rd⁴ represents a cyclic group that may include a substituent, a chain alkyl group that may include a substituent, or a chain alkenyl group that may include a substituent, each of which is the same as R′²⁰¹ described above.

Among them, an alkyl group, an alkoxy group, an alkenyl group, or a cyclic group, each of that may include a substituent, is preferred.

The alkyl group in Rd⁴ is preferably a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. A part of hydrogen atoms of the alkyl group for Rd⁴ may be replaced by a hydroxy group, a cyano group, or the like.

The alkoxy group in Rd⁴ is preferably an alkoxy group having 1 to 5 carbon atoms, and specific examples of the alkoxy group having 1 to 5 carbon atoms include a methoxy group, an ethoxy group, an n-propoxy group, an iso-propoxy group, an n-butoxy group, and a tert-butoxy group. Among them, a methoxy group and an ethoxy group are preferred.

Examples of the alkenyl group in Rd⁴ include those same as the alkenyl group in R′²⁰¹, and a vinyl group, a propenyl group (an allyl group), a 1-methylpropenyl group, and a 2-methylpropenyl group are preferred. These groups may further have an alkyl group having 1 to 5 carbon atoms or a halogenated alkyl group having 1 to 5 carbon atoms as a substituent.

Examples of the cyclic group in Rd⁴ include those same as the cyclic group in R′²⁰¹ The cyclic group in Rd⁴ is preferably an alicyclic group in which one or more hydrogen atoms are removed from a cycloalkane such as cyclopentane, cyclohexane, adamantane, norbomane, isobornane, tricyclodecane, or tetracyclododecane, or an aromatic group such as a phenyl group or a naphthyl group. When Rd⁴ is an alicyclic group, the resist composition has a good solubility in an organic solvent, and thus the lithography properties become excellent. When Rd⁴ is an aromatic group, the resist composition has an excellent light absorption efficiency, a good sensitivity, and good lithography properties in lithography using EUV or the like as a light source for exposure.

In the formula (d1-3), Yd¹ represents a single bond or a divalent linking group.

The divalent linking group in Yd¹ is not particularly limited, and examples thereof include a divalent hydrocarbon group (an aliphatic hydrocarbon group, an aromatic hydrocarbon group) that may include a substituent, and a divalent linking group containing a heteroatom.

Examples of these groups include those same as the divalent hydrocarbon group that may include a substituent and the divalent linking group containing a heteroatom, which are described in the description of the divalent linking group represented by Ya²¹ in the general formula (a2-1).

Yd′ is preferably a carbonyl group, an ester bond, an amide bond, an alkylene group, or a combination thereof. The alkylene group is more preferably a linear or branched alkylene group, and still more preferably a methylene group or an ethylene group.

Specific preferred examples of the anion moiety of the component (d1-3) are shown below.

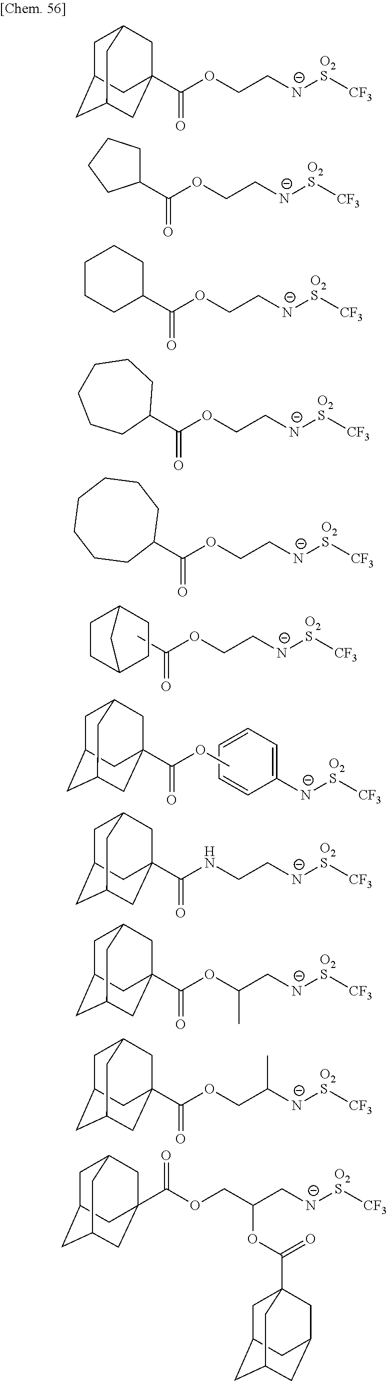

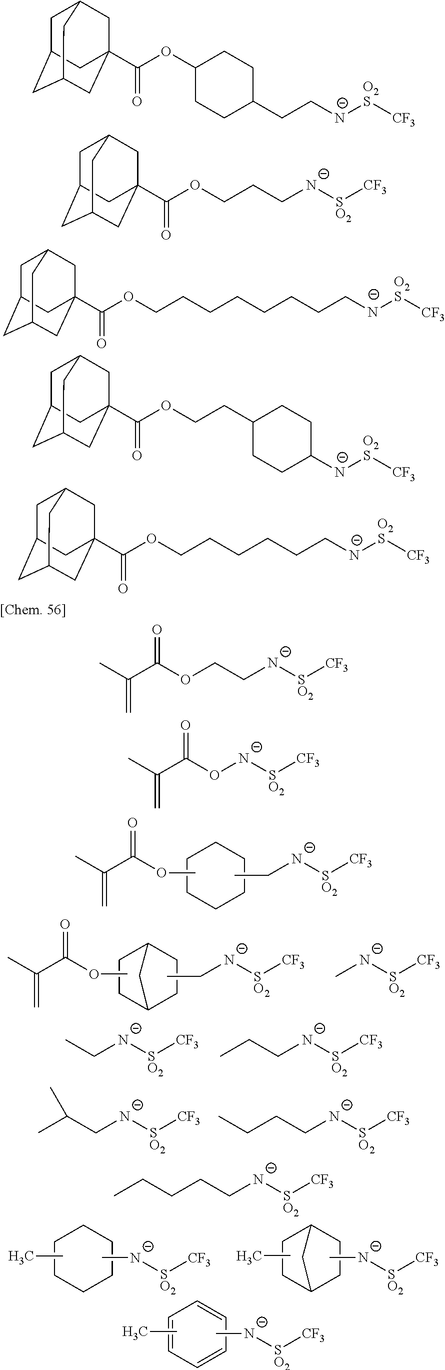

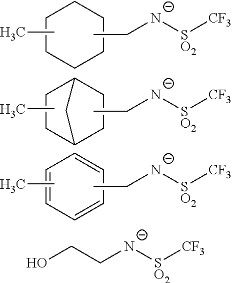

### Cation Moiety

In the formula (d1-3), M^{m+} represents an m-valent organic cation, and is the same as M^{m+} in the formula (d1-1).

The component (d1-3) may be used alone or in combination of two or more kinds thereof.

As the component (D1), only one kind of the components (d1-1) to (d1-3) may be used, or two or more kinds thereof may be used in combination.

When the resist composition contains the component (D1), a content of the component (D1) in the resist composition is preferably 0.5 parts by mass to 25 parts by mass, more preferably 1 part by mass to 20 parts by mass, and still more preferably 2.5 parts by mass to 15 parts by mass, with respect to 100 parts by mass of the component (A).

When the content of the component (D1) is equal to or larger than the preferred lower limit value, particularly good lithography properties and a particularly good resist pattern shape are easily obtained. On the other hand, when the content of the component (D1) is equal to or less than an upper limit value, the sensitivity can be favorably maintained and the throughput is also excellent.

### Method for Producing Component (D1):

A method for producing the component (d1-1) and the component (d1-2) is not particularly limited, and the component (d1-1) and the component (d1-2) can be produced by known methods.

A method for producing the component (d1-3) is not particularly limited, and the component (d1-3) can be produced, for example, in the same manner as a method described in US2012-0149916.

### Regarding Component (D2)

The acid diffusion control agent component may contain a nitrogen-containing organic compound component (hereinafter, referred to as “component (D2)”) not corresponding to the component (D1).

The component (D2) is not particularly limited as long as it acts as an acid diffusion control agent and does not correspond to the component (D1), and any known compounds may be used. Among them, an aliphatic amine or an aromatic amine is preferred, and an aromatic amine is more preferred.

The aliphatic amine is an amine having one or more aliphatic groups, and the aliphatic group preferably has 1 to 12 carbon atoms.

Examples of the aliphatic amine include an amine in which at least one hydrogen atom of ammonia NH₃ is replaced by an alkyl group or a hydroxyalkyl group having 12 or less carbon atoms (an alkyl amine or an alkyl alcohol amine), and a cyclic amine.

Specific examples of the alkyl amine and the alkyl alcohol amine include monoalkylamines such as n-hexylamine, n-heptylamine, n-octylamine, n-nonylamine, and n-decylamine; dialkylamines such as diethylamine, di-n-propylamine, di-n-heptylamine, di-n-octylamine, and dicyclohexylamine; trialkylamines such as trimethylamine, triethylamine, tri-n-propylamine, tri-n-butylamine, tri-n-pentylamine, tri-n-hexylamine, tri-n-heptylamine, tri-n-octylamine, tri-n-nonylamine, tri-n-decylamine, and tri-n-dodecylamine; and alkyl alcohol amines such as diethanolamine, triethanolamine, diisopropanolamine, triisopropanolamine, di-n-octanolamine, and tri-n-octanolamine. Among them, trialkylamine having 6 to 30 carbon atoms is more preferred, and tri-n-pentyl amine or tri-n-octyl amine is particularly preferred.

Examples of the cyclic amine include a heterocyclic compound containing a nitrogen atom as a heteroatom. The heterocyclic compound may be a monocyclic compound (an aliphatic monocyclic amine) or a polycyclic compound (an aliphatic polycyclic amine).

Specific examples of the aliphatic monocyclic amine include piperidine and piperazine.

The aliphatic polycyclic amine preferably has 6 to 10 carbon atoms, and specific examples thereof include 1,5-diazabicyclo[4.3.0]-5-nonene, 1,8-diazabicyclo[5.4.0]-7-undecene, hexamethylenetetramine, and 1,4-diazabicyclo[2.2.2]octane.

Examples of other aliphatic amines include tris(2-methoxymethoxyethyl)amine, tris{2-(2-methoxyethoxy)ethyl}amine, tris{2-(2-methoxyethoxymethoxy)ethyl}amine, tris{2-(1-methoxyethoxy)ethyl}amine, tris{2-(1-ethoxyethoxy)ethyl}amine, tris{2-(1-ethoxypropoxy)ethyl}amine, tris[2-{2-(2-hydroxyethoxy)ethoxy}ethyl]amine, and triethanolamine triacetate. Triethanolamine triacetate is preferred.

As the component (D2), an aromatic amine may be used.

Examples of the aromatic amine include 4-dimethylaminopyridine, 2,6-di-tert-butylpyridine, pyrrole, indole, pyrazole, imidazole or derivatives thereof, tribenzylamine, 2,6-diisopropylaniline, and N-tert-butoxycarbonylpyrrolidine.

The component (D2) may be used alone or in combination of two or more kinds thereof.

When the resist composition contains the component (D2), a content of the component (D2) in the resist composition is generally in a range of 0.01 parts by mass to 5 parts by mass with respect to 100 parts by mass of the component (A). When the content is within the range, the resist pattern shape and the storage stability over time are improved.

<<Component (B): At Least One Compound Selected from Group Consisting of Organic Carboxylic Acid, and Phosphorus Oxoacid and Derivative Thereof>>

The resist composition according to the embodiment of the present invention may contain at least one compound (E) (hereinafter, referred to as “component (E)”) selected from the group consisting of an organic carboxylic acid, and a phosphorus oxoacid and a derivative thereof as an optional component for the purpose of preventing deterioration in sensitivity, and improving the shape of a resist pattern, the storage stability over time, and the like.

Suitable examples of the organic carboxylic acid include acetic acid, malonic acid, citric acid, malic acid, succinic acid, benzoic acid, hydroxybenzoic acid, salicylic acid, phthalic acid, terephthalic acid, and isophthalic acid.

Examples of the phosphorus oxoacid include phosphoric acid, phosphonic acid, and phosphinic acid. Among them, phosphonic acid is particularly preferred.

Examples of the derivative of phosphorus oxoacid include an ester in which a hydrogen atom of an oxo acid is replaced by a hydrocarbon group, and examples of the hydrocarbon group include an alkyl group having 1 to 5 carbon atoms and an aryl group having 6 to 15 carbon atoms.

Examples of the derivative of phosphoric acid include phosphate esters such as di-n-butyl phosphate and diphenyl phosphate.

Examples of the derivative of phosphonic acid include phosphonate esters such as dimethyl phosphonate, di-n-butyl phosphonate, phenyl phosphonic acid, diphenyl phosphonate, and dibenzyl phosphonate.

Examples of the derivative of phosphinic acid include phosphinate esters and phenylphosphinic acid.

Among them, the component (B) is preferably an organic carboxylic acid, and more preferably an aromatic carboxylic acid. Specifically, benzoic acid, hydroxybenzoic acid, salicylic acid, phthalic acid, terephthalic acid, and isophthalic acid are preferred. Among them, salicylic acid is more preferred.

In the resist composition according to the embodiment of the present invention, the component (E) may be used alone or in combination of two or more kinds thereof.

When the resist composition contains the component (E), a content of the component (B) is preferably 0.01 parts by mass to 5 parts by mass, more preferably 0.1 parts by mass to 5 parts by mass, and still more preferably 0.1 parts by mass to 3 parts by mass, with respect to 100 parts by mass of the component (A).

### <<Component (F): Fluorine Additive Component>

The resist composition of the present embodiment may contain a fluorine additive component (hereinafter, referred to as “component (F)”) as a hydrophobic resin. The component (F) is used to impart water-repellent to the resist film. When the component (F) is used as a resin different from the component (A), the lithography properties are improved.

As the component (F), for example, fluorinated polymer compounds described in JP2010-002870A, JP2010-032994A, JP2010-277043A, JP2011-13569A, and JP2011-128226A can be used.

Specific examples of the component (F) include a polymer having a structural unit (f11) represented by the following general formula (f1-1) or a structural unit (f12) represented by the following general formula (f1-2).

The polymer having a structural unit (f11) represented by the following general formula (f1-1) is preferably a polymer (homopolymer) consisting only of a structural unit (f11) represented by the following general formula (f1-1); a copolymer of the structural unit (f11) and the structural unit (a1); or a copolymer of the structural unit (f11), a structural unit derived from acrylic acid or methacrylic acid, and the structural unit (a1). Here, as the structural unit (a1) to be copolymerized with the structural unit (f11), a structural unit containing an acid-dissociable group represented by the formula (a1-r2-1) is preferred.

Examples of the polymer having a structural unit (f12) represented by the following general formula (f1-2) include a polymer (homopolymer) consisting only of a structural unit (f12) represented by the following general formula (f1-2), and a copolymer of the structural unit (f12) and the structural unit (a1). Among them, a copolymer of the structural unit (f12) and the structural unit (a1) is preferred.

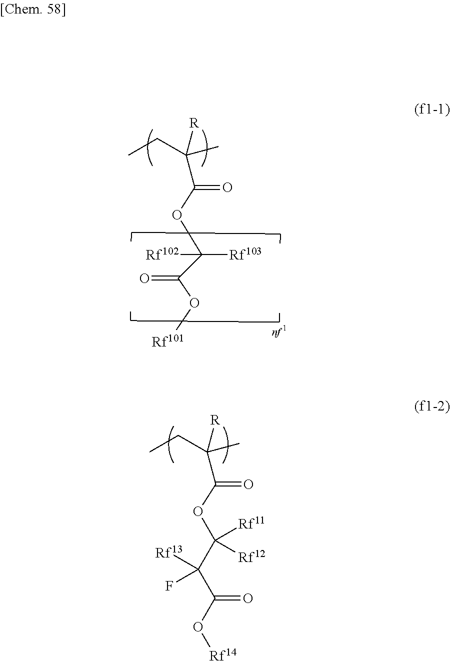

[In the formula, R is the same as R in the general formula (a1-1). Rf¹⁰² and Rf¹⁰³ each independently represent a hydrogen atom, a halogen atom, an alkyl group having 1 to 5 carbon atoms, or a halogenated alkyl group having 1 to 5 carbon atoms, and Rf¹⁰² and Rf¹⁰³ may be the same as or different from each other. The symbol nf¹ is an integer of 0 to 5, and Rf¹⁰¹ is an organic group containing a fluorine atom. Rf¹¹ and Rf¹² each independently represent a hydrogen atom, an alkyl group having 1 to 4 carbon atoms, or a fluorinated alkyl group having 1 to 4 carbon atoms. Rf¹³ is a fluorine atom or a fluorinated alkyl group having 1 to 4 carbon atoms. Rf¹⁴ is a linear or branched alkyl group having 1 to 4 carbon atoms, or a linear fluorinated alkyl group having 1 to 4 carbon atoms.]

In the general formula (f1-1). R bonded to a carbon atom at an α-position is the same as defined above. R is preferably a hydrogen atom or a methyl group.

Examples of the halogen atom of Rf¹⁰² and Rf¹⁰³ in the general formula (f1-1) include a fluorine atom, a chlorine atom, a bromine atom, and an iodine atom, and a fluorine atom is particularly preferred. Examples of the alkyl group having 1 to 5 carbon atoms for Rf¹⁰² and Rf¹⁰³ include those same as the alkyl group having 1 to 5 carbon atoms for the above R, and a methyl group or an ethyl group is preferred. Specific examples of the halogenated alkyl group having 1 to 5 carbon atoms for Rf¹⁰² and Rf¹⁰³ include a group in which a part or all of hydrogen atoms of the alkyl group having 1 to 5 carbon atoms are replaced by halogen atoms. Examples of the halogen atom include a fluorine atom, a chlorine atom, a bromine atom, and an iodine atom. A fluorine atom is particularly preferred. Among them, as Rf¹⁰² and Rf¹⁰³, a hydrogen atom, a fluorine atom, or an alkyl group having 1 to 5 carbon atoms is preferred, and a hydrogen atom, a fluorine atom, a methyl group, or an ethyl group is preferred.

In the general formula (f1-1), n^{f1} is preferably an integer of 1 to 5, more preferably an integer of 1 to 3, and further preferably 1 or 2.

In the general formula (f1-1), Rf¹⁰¹ represents an organic group containing a fluorine atom, and is preferably a hydrocarbon group containing a fluorine atom.

The hydrocarbon group containing a fluorine atom may be any of a linear group, branched group, and cyclic group, and preferably has 1 to 20 carbon atoms, more preferably has 1 to 15 carbon atoms, and particularly preferably has 1 to 10 carbon atoms.

In the hydrocarbon group containing a fluorine atom, it is preferable that 25% or more of hydrogen atoms in the hydrocarbon group be fluorinated, it is more preferable that 50% or more of hydrogen atoms be fluorinated, and it is particularly preferable that 60% or more of hydrogen atoms be fluorinated, since hydrophobicity of the resist film during immersion exposure is enhanced.

Among them, Rf¹⁰¹ is more preferably a fluorinated hydrocarbon group having 1 to 6 carbon atoms, still more preferably a trifluoromethyl group, —CH₂—CF₃, —CH₂—CF₃—CF₃, —CH(CF₃)₂, —CH₂—CH₂—CF₃, or —CH₂—CH₂—CF₂—CF₂—CF₂—CF₃, and particularly preferably —CH₂—CF₃.

In the general formula (f1-2), R bonded to a carbon atom at an α-position is the same as defined above. R is preferably a hydrogen atom or a methyl group.

In the general formula (f1-2), Rf¹¹ and Rf¹² each independently represent a hydrogen atom, an alkyl group having 1 to 4 carbon atoms, or a fluorinated alkyl group having 1 to 4 carbon atoms.

The alkyl group having 1 to 4 carbon atoms in Rf¹¹ and Rf¹² may be any of a linear group, branched group, and cyclic group, and is preferably a linear or branched alkyl group. Preferred examples thereof include a methyl group and an ethyl group, and an ethyl group is particularly preferred.

The fluorinated alkyl group having 1 to 4 carbon atoms in Rf¹¹ and Rf¹² is a group in which a part or all of hydrogen atoms in the alkyl group having 1 to 4 carbon atoms are replaced by fluorine atoms. In the fluorinated alkyl group, the alkyl group that is not replaced by a fluorine atom may be any of a linear group, branched group, and cyclic group, and examples thereof include those same as the “alkyl group having 1 to 4 carbon atoms in Rf¹¹ and Rf¹²”.

Among them, Rf¹¹ and Rf¹² preferably represent a hydrogen atom or an alkyl group having 1 to 4 carbon atoms, and it is particularly preferable that one of Rf¹¹ and Rf¹² be a hydrogen atom and the other be an alkyl group having 1 to 4 carbon atoms.

In the general formula (f1-2), Rf¹³ represents a fluorine atom or a fluorinated alkyl group having 1 to 4 carbon atoms.

Examples of the fluorinated alkyl group having 1 to 4 carbon atoms in Rf¹³ include those same as the “fluorinated alkyl group having 1 to 4 carbon atoms in Rf¹¹ and Rf¹²”, and the number of the carbon atoms thereof is preferably 1 to 3, and more preferably 1 or 2 carbon atoms.

In the fluorinated alkyl group for Rf¹³, a proportion (a fluorination ratio (%)) of the number of fluorine atoms to the total number of fluorine atoms and hydrogen atoms contained in the fluorinated alkyl group is preferably 30% to 100%, and more preferably 50% to 100% The higher the fluorination ratio, the higher the hydrophobicity of the resist film.

Among them, Rf¹³ is preferably a fluorine atom.

In the general formula (f1-2), Rf¹⁴ represents a linear or branched alkyl group having 1 to 4 carbon atoms or a linear fluorinated alkyl group having 1 to 4 carbon atoms, and is preferably a linear alkyl group having 1 to 4 carbon atoms or a linear fluorinated alkyl group having 1 to 4 carbon atoms.

Specific examples of the alkyl group in Rf¹⁴ include a methyl group, an ethyl group, a propyl group, an isopropyl group, an n-butyl group, an isobutyl group, and a tert-butyl group.

Among them, a methyl group and an ethyl group are preferred, and a methyl group is most preferred.

Specific examples of the fluorinated alkyl group in Rf¹⁴ include —CH₂—CF₃, —CH₂—CH₂—CF₃, —CH₂—CF₂—CF₃, and —CH₂—CF₂—CF₂—CF₃, and among them, —CH₂—CH₂—CF₃ is particularly preferred.

A weight average molecular weight (Mw) of the component (F) (based on polystyrene equivalent determined by gel permeation chromatography) is preferably 1000 to 50000, more preferably 5000 to 40000, and most preferably 10000 to 30000. When the weight average molecular weight (Mw) is equal to or less than an upper limit value of this range, sufficient solubility in a resist solvent for use as a resist is obtained. When the weight average molecular weight (Mw) is equal to or larger than a lower limit value of this range, dry etching resistance and a cross-sectional shape of the resist pattern are good.

A dispersity (Mw/Mn) of the component (F) is preferably 1.0 to 5.0, more preferably 1.0 to 3.0, and most preferably 1.0 to 2.5.

In the resist composition of the present embodiment, the component (F) may be used alone or in combination of two or more kinds thereof.

When the resist composition contains the component (F), as a content of the component (F), the component (F) is generally used in a proportion of 0.5 parts by mass to 10 parts by mass with respect to 100 parts by mass of the component (A).

### <<Component(S): Organic Solvent Component>>

The resist composition according to the present embodiment can be produced by dissolving a resist material in an organic solvent component (hereinafter, referred to as “component (S)”).

As the component (S), those capable of dissolving components to be used to form a uniform solution may be used, and any one can be appropriately selected from those are known in the related art as a solvent for a chemically amplified resist composition. In the resist composition of the present embodiment, the component (S) may be used alone or as a mixed solvent of two or more kinds thereof.

Among the above, propylene glycol monomethyl ether acetate (PGMEA), propylene glycol monomethyl ether (PGMB), γ-butyrolactone, propylene carbonate, ethyl lactate (EL), and cyclohexanone are preferred, and PGMEA, PGME, and cyclohexanone are more preferred.

A mixed solvent of PGMEA and a polar solvent is also preferred. A blending ratio (a mass ratio) thereof may be appropriately determined taking into consideration the compatibility between PGMEA and the polar solvent, and is preferably within a range of 1:9 to 9:1, and more preferably a range of 2:8 to 8:2.

More specifically, when EL or cyclohexanone is blended as the polar solvent, the mass ratio of PGMEA to BL or cyclohexanone is preferably 1:9 to 9:1, and more preferably 2:8 to 8:2. When PGME is blended as the polar solvent, the mass ratio of PGMEA to PGME is preferably 1:9 to 9:1, more preferably 2:8 to 8:2, and still more preferably 3:7 to 7:3.

As the component (S), a mixed solvent of at least one selected from PGMEA and EL and at least one selected from γ-butyrolactone and propylene carbonate is also preferred. In this case, as the mixing proportion, the mass ratio of the former to the latter is preferably 60:40 to 99:1, and more preferably 70:30 to 95:5.

An amount of the component (S) to be used is not particularly limited, and is appropriately set according to a coating film thickness at a concentration such that the component (S) can be applied to a substrate or the like. Generally, the component (S) is used such that a solid content concentration of the resist composition is in a range of 0.1 mass % to 20 mass %, and preferably 0.2 mass % to 15 mass %.

The resist composition according to the present embodiment may further contain, if desired, compatible additives such as an additional resin, a dissolution inhibitor, a plasticizer, a stabilizer, a colorant, an antihalation agent, or a dye for improving the performance of the resist film.

The resist composition according to the embodiment of the present invention contains the component (A) and component (B), and, if necessary, the optional component.

A resist composition containing the component (A), the component (B), and the component (D) is suitable. Further, a resist composition containing the component (A), the component (B), the component (D), and the component (F) is suitable.

As described above, the resist composition according to the embodiment of the present invention contains the base material component (A) and the acid generating agent component (B). When the acid generating agent component (B) in the resist composition contains the compound (b1-1) having a specific structure, the anion moiety can be imparted with stability against acid and base and moderate bulkiness. As a result, it is presumed that when the reaction with other components in the composition is prevented, diffusion during baking is controlled to an appropriate range at the same time, whereby a resist pattern excellent in stability over time and having both a high exposure latitude and a high sensitivity to a light source for exposure can be formed.

### (Method for Forming Resist Pattern)

A method for forming a resist pattern according to a second aspect of the present invention is a method including: a step of forming a resist film on a support using the resist composition of the embodiment; a step of exposing the resist film, and a step of developing the resist film to form a resist pattern.

One embodiment of the method for forming a resist pattern includes, for example, a method for forming a resist pattern performed as follows.

First, the resist composition of the embodiment is applied onto a support using a spinner or the like, and a baking treatment (a post apply bake (PAB) is performed, for example, for 40 seconds to 120 seconds, preferably 50 seconds to 90 seconds under a temperature condition of 80° C. to 150° C. to form a resist film.

Next, the resist film is selectively exposed through a mask (a mask pattern) on which a predetermined pattern is formed, or by direct irradiation with an electron beam without a mask pattern using an exposure apparatus such as an electron beam drawing device or an EUV exposure apparatus, and then a baking treatment (a post-exposure bake (PEB)) is performed, for example, for 40 seconds to 120 seconds, preferably 50 seconds to 90 seconds under a temperature condition of 80° C. to 150° C.

Next, the resist film is subjected to a developing treatment. The developing treatment is performed using an alkaline liquid developer in the case of an alkaline developing process, and using a liquid developer containing an organic solvent (an organic liquid developer) in the case of a solvent developing process.

After the developing treatment, a rinsing treatment is preferably performed. As the rinsing treatment, water rinsing using pure water is preferred in the case of the alkaline developing process, and a rinsing liquid containing an organic solvent is preferably used in the case of the solvent developing process.

In the case of the solvent developing process, after the developing treatment or the rinsing treatment, a process of removing a liquid developer or a rinsing liquid attached to the pattern by a supercritical fluid may be performed.

After the developing treatment or the rinsing treatment, drying is performed. In some cases, a baking treatment (a post bake) may be performed after the developing treatment.

The support is not particularly limited, and any related-art known supports can be used. Examples thereof include a substrate for electronic components or a substrate having a predetermined wiring pattern formed thereon. More specifically, a silicon wafer, a substrate made of a metal such as copper, chromium, iron, or aluminum, a glass substrate, or the like may be used. As a material of the wiring pattern, for example, copper, aluminum, nickel, or gold can be used.

A wavelength used for exposure is not particularly limited, and an ArF excimer laser, a KrF excimer laser, an F2 excimer laser, extreme ultraviolet (EUV), vacuum ultraviolet (VUV), electron beam (BB), and radiation rays such as X-rays and soft X-rays can be used.

A resist film exposure method may be general exposure (dry exposure) performed in an inert gas such as air or nitrogen, or may be liquid immersion lithography, and liquid immersion lithography is preferred.

The liquid immersion lithography is an exposure method in which a space between a resist film and a lowermost lens of an exposure apparatus is filled with a solvent (a liquid immersion medium) having a refractive index greater than that of air, and exposure (immersion exposure) is then performed in that state.

As the liquid immersion medium, a solvent having a refractive index larger than that of air and smaller than that of a resist film to be exposed is preferred. The refractive index of such a solvent is not particularly limited as long as it is within the range.

Examples of the solvent having a refractive index larger than that of air and smaller than that of the resist film include water, a fluorine inert liquid, a silicon solvent, and a hydrocarbon solvent.

Water is preferably used as the liquid immersion medium.

Examples of the alkaline liquid developer used in the developing treatment in the alkaline developing process include a 0.1 mass % to 10 mass % aqueous solution of tetramethylammonium hydroxide (TMAH).

The organic solvent contained in the organic liquid developer used in the developing treatment in the solvent developing process may be any solvents capable of dissolving the component (A) (the component (A) before exposure), and may be appropriately selected from known organic solvents. Specific examples thereof include polar solvents such as a ketone solvent, an ester solvent, an alcohol solvent, a nitrile solvent, an amide solvent, and an ether solvent, and hydrocarbon solvents.

The ketone solvent is an organic solvent containing C—C(═O)—C or C—C(═O)—H in a structure thereof. The ester solvent is an organic solvent containing C—C(═O)—O—C or H—C(═O)—O—C in a structure thereof. The alcohol solvent is an organic solvent containing an alcoholic hydroxy group in a structure thereof. The term “alcoholic hydroxy group” refers to a hydroxy group bonded to a carbon atom of an aliphatic hydrocarbon group. The nitrile solvent is an organic solvent containing a nitrile group in a structure thereof. The amide solvent is an organic solvent containing an amide group in a structure thereof. The ether solvent is an organic solvent containing C—O—C in a structure thereof.

Among the organic solvents, there is an organic solvent containing a plurality of kinds of functional groups characterizing each of the solvents in the structure, and in this case, the organic solvent corresponds to any kind of solvent containing the functional group of the organic solvent. For example, diethylene glycol monomethyl ether corresponds to both the alcohol solvent and the ether solvent in the classification.

The hydrocarbon solvent is a hydrocarbon solvent composed of a hydrocarbon that may be halogenated and having no substituent other than a halogen atom. Examples of the halogen atom include a fluorine atom, a chlorine atom, a bromine atom, and an iodine atom. A fluorine atom is preferred.

Among the solvents described above, the organic solvent contained in the organic liquid developer is preferably the polar solvent, and is preferably the ketone solvent, the ester solvent, the nitrile solvent, or the like.

Examples of the ester solvent include methyl acetate, butyl acetate, and ethyl acetate.

Examples of the nitrile solvent include acetonitrile, propionitrile, valeronitrile, and butyrnitrile.

If necessary, known additives may be added to the organic liquid developer. Examples of the additive include a surfactant. The surfactant is not particularly limited, and for example, ionic or nonionic fluorine and/or silicon surfactants can be used.

The developing treatment can be performed by a known developing method, and examples thereof include a method in which a support is immersed with respect to a liquid developer for a certain period of time (a dip method), a method in which a liquid developer is piled up on a surface of a support by surface tension and left standing for a certain period of time (a paddle method), a method in which a liquid developer is sprayed onto a surface of a support (a spray method), a method in which a liquid developer is continuously dispensed by scanning a liquid developer dispense nozzle at a constant speed onto a support that is rotating at a constant speed (a dynamic dispense method).

As the organic solvent contained in the rinsing liquid used in the rinsing treatment after the developing treatment in the solvent developing process, for example, an organic solvent which hardly dissolves the resist pattern can be appropriately selected and used among the organic solvents described above as the organic solvent used in the organic liquid developer. Generally, at least one solvent selected from a hydrocarbon solvent, a ketone solvent, an ester solvent, an alcohol solvent, an amide solvent, and an ether solvent is used.

These organic solvents may be used alone or in combination of two or more kinds thereof. In addition, an organic solvent other than those described above or water may be mixed and used.

The rinsing treatment (a washing treatment) using the rinsing liquid can be performed by a known rinsing method. Examples of the method of the rinsing treatment include a method in which a rinsing liquid is continuously dispensed onto a support that is rotating at a constant speed (a spin dispensing method), a method in which a support is immersed in a rinsing liquid for a certain period of time (a dip method), and a method in which a rinsing liquid is sprayed onto a surface of a support (a spray method).

Various materials used in the resist composition of the embodiment and the method for forming a pattern of the embodiment (for example, a resist solvent, a liquid developer, a rinsing liquid, a composition for forming an antireflection film, a composition for forming a top coat, and the like) preferably does not contain impurities such as a metal, a metal salt containing a halogen, an acid, an alkali, and a component containing a sulfur atom or a phosphorus atom. Here, examples of the impurity containing a metal atom include Na, K, Ca, Fe, Cu, Mn, Mg, Al, Cr, Ni, Zn, Ag, Sn, Pb, Li, and salts thereof. A content of the impurities contained in these materials is preferably 200 ppb or less, more preferably 1 ppb or less, still more preferably 100 ppt (parts per trillion) or less, and particularly preferably 10 ppt or less, and it is most preferable that the impurity be not substantially contained (equal to or less than a detection limit of a measuring device).

In the method for forming a resist pattern according to the embodiment of the present invention described above, the resist composition according to the embodiment of the present invention is used, and therefore a resist pattern having both a high exposure latitude and a high sensitivity to the exposure light source can be formed.

### [Salt]

A salt according to a third aspect of the present invention is a salt represented by the general formula (b1-1) (hereinafter, referred to as “compound (b1-1)”).

The compound (b1-1) is the same as the component (BI) in the resist composition according to a first aspect of the present invention, and preferred examples are also the same.

The polycyclic hydrocarbon group represented by Y in the general formula (b1-1) preferably has an adamantane skeleton or a norbornane skeleton.

It is preferable that M^{m+} in the general formula (b1-1) be a cation represented by the above general formula (b-2).

The compound (b1-1) can be produced by a standard method.

As a specific example, when the divalent polycyclic hydrocarbon group represented by Y in the compound (b1-1) is a group having an adamantane skeleton, the linking group represented by L is an ester bond (—CO—O—), and M^{m+} is triphenylsulfonium, the compound (hereinafter, referred to as “compound (b1-1-4)”) can be produced as follows, via an intermediate compound (the following compound (b1-1-3)) in which M^{m+} is not triphenylsulfonium.

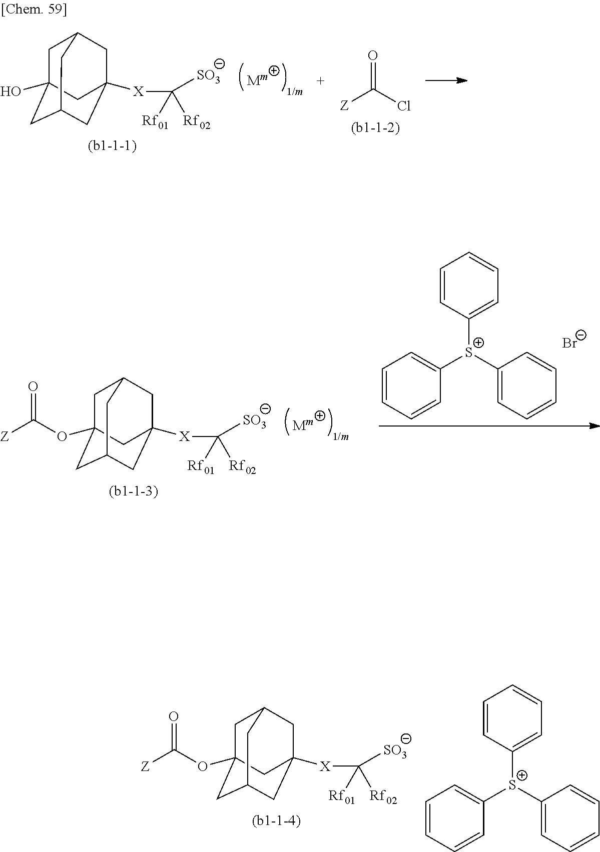

[In the general formula (b1-1-1) to (b1-1-4), Rf₀₁ and Rf₀₂ each independently represent a hydrogen atom, a fluorine atom, or a fluorinated alkyl group, and at least one of Rf⁰¹ and Rf₀₂ contains a fluorine atom. X represents a divalent linking group having a total of 4 or more atoms in the main chain, and the divalent linking group represents an alkylene group which may contain an ester bond or an ether bond, a fluorinated alkylene group which may contain an ester bond or an ether bond, or a combination thereof. Any of ester bonds and ether bonds in X does not form an acetal bond adjacent to each other. Z represents a substituent represented by the above formula (bz-1), m represents an integer of 1 or more, and M^{m+} represents an m-valent organic cation.]

Rf₀₁, Rf₀₂, X, Z, m and M^{m+} in the general formulae (b1-1-1) to (b1-1-4) have the same meanings as Rf₀₁, Rf₀₂, X, Z, m and M^{m+} in the general formula (b1-1), and preferred Rf₀₁, Rf₀₂, X, Z, m and M^{m+} in the general formulae (b1-1-1) to (b1-1-4) are the same as preferred Rf₀₁, Rf₀₂, X, Z, m and M^{m+} in the general formula (b1-1).

First, the compound represented by the general formula (b1-1-1) is added to an organic solvent (for example, acetonitrile, acetone, dichloromethane, tetrahydrofuran, etc.) and cooled, and a solution prepared by dissolving the compound represented by the general formula (b1-1-2) in an organic solvent in the presence of a base (triethylamine, pyridine, 4-(dimethylamino)pyridine, etc.) is added thereto to react, and after separation and washing with water, the compound represented by the general formula (b1-1-3) is obtained from the organic phase.

When further salt exchange is performed, the compound (b1-1-3) is dissolved in a mixed solvent of an organic solvent (for example, dichloromethane and tetrahydrofuran) and water, a compound containing a desired cation (in the above example, triphenylsulfonium bromide) is added thereto to react, and after separation and washing with water, the compound (b1-1-4) is collected from the organic phase.

As described above, among the salts represented by the general formula (b1-1), a salt in which the organic cation in M^{m+} is an ammonium cation is also useful as a synthetic intermediate for a salt in which the organic cation in M^{m+} is a sulfonium cation or an iodonium cation.

The structure of the obtained compound can be confirmed by a general organic analysis method such as ¹H-nuclear magnetic resonance (NMR) spectroscopy, ¹³C-NMR spectroscopy, ¹⁹F-NMR spectroscopy, infrared absorption (IR) spectroscopy, mass spectrometry (MS), elemental analysis, or X-ray crystal diffraction.

The above compound (b1-1) is a novel compound that is not known.

The compound (b1-1) is a novel compound useful as an acid generating agent for a resist composition, and can be blended in the resist composition as an acid generating agent.

The compound represented by the general formula (b1-1) is useful as an acid generating agent.

An acid generating agent composed of the compound represented by general formula (b1-1) is useful as an acid generating agent for a chemically amplified resist composition, for example, as the acid generating agent component (B) of the resist composition according to the first aspect of the present invention.

### EXAMPLES

The present invention will be described in more detail below with reference to Examples, but the present invention is not limited to these Examples.

### <Synthesis of Acid Generating Agent Component (B)>

In the present Examples, compounds used as the acid generating agent component (B) were synthesized according to synthesis examples shown below.

### Synthesis Example of Compound (B-1)

A compound represented by a formula (1-1) (30 g), acetonitrile (130 g), and triethylamine (9.2 g) were mixed, and a solution of cyclohexanecarbonyl chloride (10.6 g) in acetonitrile (20 g) was added dropwise thereto, followed by stirring at room temperature for 48 hours. The resulting reaction solution was filtered, and the filtrate was concentrated to obtain a residue.

The residue was dissolved in dichloromethane (150 g), then water (200 g) was added, and the mixture was stirred at room temperature for 30 minutes, and the organic phase was separated. The water washing operation was repeated five times.

The collected organic phase was concentrated to obtain a salt (27 g) represented by a formula (B1-1-1).

The salt represented by the formula (B1-1-1) (20 g), dichloromethane (200 g), triphenylsulfonium bromide (a formula (2-1)) (14.9 g), and ion-exchanged water (115 g) were mixed and stirred at room temperature for 30 minutes, and then the organic phase was separated. Next, ion-exchanged water (115 g) was added to the separated organic phase, the mixture was stirred at room temperature for 30 minutes, and then the organic phase was separated. The water washing operation was repeated eight times. Next, the obtained organic phase was concentrated to obtain a salt represented by a formula (B-1) (a compound (B-1)) (21 g).

The compound (B-1) was analyzed by NMR.

¹H NMR (400 MHz, DMSO-d6) δ (ppm)=7.93-7.69 (m, 15H), 4.97 (m, 1H), 4.30-4.05 (m, 2H), 2.46-1.40 (m, 22H), 1.40-1.10 (m, 5H).

From the above results, it was confirmed that the compound (B-1) had the following structure.

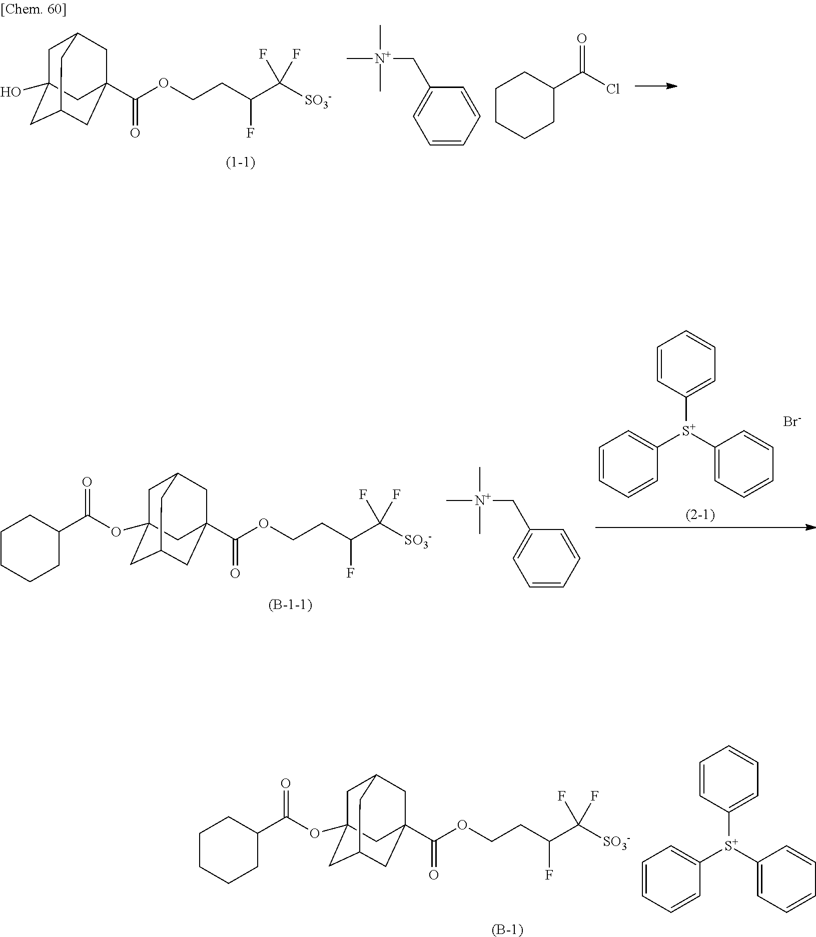

### Synthesis Example of Compound (B-2)

The same operation was performed as in the synthesis of the above compound (B-1), except that cyclohexanecarbonyl chloride (10.6 g) was replaced with cyclopentanecarbonyl chloride (9.6 g) to synthesize. Thus, a compound (B-2) was obtained.

The compound (B-2) was analyzed by NMR.

¹H NMR (400 MHz, DMSO-d6) δ (ppm)=7.93-7.69 (m, 15H), 4.97 (m, 1H), 4.30-4.05 (m, 2H), 2.46-1.10 (m, 25H).

### Synthesis Example of Compound (B-3)

The same operation was performed as in the synthesis of the above compound (B-1), except that cyclohexanecarbonyl chloride (10.6 g) was replaced with 4-tert-butylcyclohexanecarbonyl chloride (14.7 g) to synthesize. Thus, a compound (B-3) was obtained.

The compound (B-3) was analyzed by NMR.

¹H NMR (400 MHz, DMSO-d6) δ (ppm)=7.93-7.69 (m, ISH), 4.97 (m, 1H), 4.30-4.05 (m, 2H), 2.46-0.62 (m, 35H).

### Synthesis Example of Compound (B-4)

The same operation was performed as in the synthesis of the above compound (B-1), except that cyclohexane carbonyl chloride (10.6 g) was replaced with 3-methoxycyclohexane carbonyl chloride (12.8 g) to synthesize. Thus, a compound (B-4) was obtained.

The compound (B-4) was analyzed by NMR.

¹H NMR (400 MHz, DMSO-d6) δ (ppm)=7.93-7.69 (m, 15H), 4.97 (m, 1H), 4.30-4.05 (m, 2H), 3.65-1.10 (m, 29H).

### Synthesis Example of Compound (B-5)

The same operation was performed as in the synthesis of the above compound (B-1), except that the compound represented by the above formula (1-1) (30 g) was replaced with a compound represented by the following formula (1-2) (30.7 g) to synthesize. Thus, a compound (B-5) was obtained.

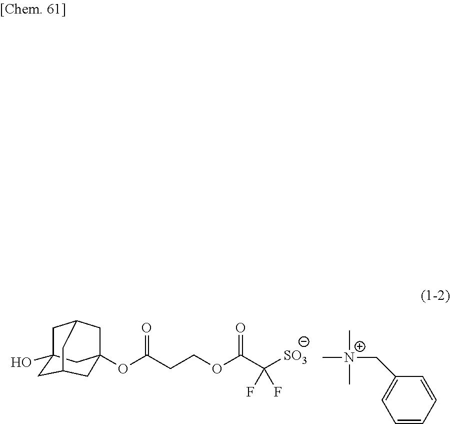

The compound (B-5) was analyzed by NMR.

¹H NMR (400 MHz, DMSO-d6) δ (ppm)=7.93-7.69 (m, 15H), 4.63-4.32 (m, 2H), 2.80-2.55 (m, 2H), 2.46-1.10 (m, 25H).

### Synthesis Example of Compound (B-6)

The same operation was performed as in the synthesis of the compound (B-1), except that the compound represented by the formula (1-1) (30 g) was replaced with a compound represented by the following formula (1-3) (31.0 g) to synthesize. Thus, a compound (B-6) was obtained.

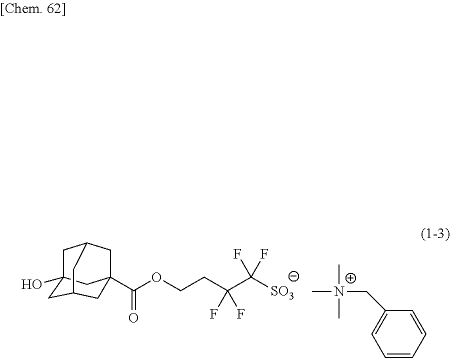

The compound (B-6) was analyzed by NMR.

¹H NMR (400 MHz, DMSO-d6) δ (ppm)=7.93-7.69 (m, 15H), 4.31-4.19 (m, 2H), 2.65-1.10 (m, 27H).

### Synthesis Example of Compound (B-7)

The same operation was performed as in the synthesis of the compound (B-1), except that triphenylsulfonium bromide (the formula (2-1)) (14.9 g) was replaced with a compound represented by the following formula (2-2) (15.5 g) to synthesize. Thus, a compound (B-7) was obtained.

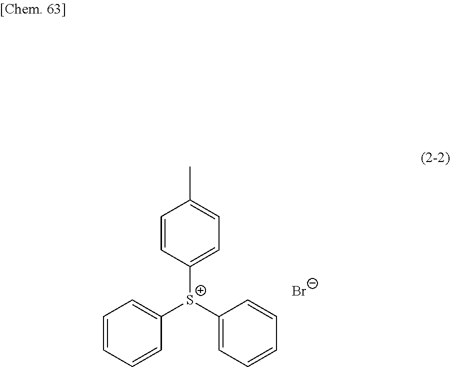 [SMILES: Cc1ccc([S+](c2ccccc2)c2ccccc2)cc1 ; [Br-]]

The compound (B-7) was analyzed by NMR.

¹H NMR (400 MHz, DMSO-d6) δ (ppm)=7.93-7.61 (m, 14H), 4.97 (m, 1H), 4.30-4.05 (m, 2H), 2.48-1.40 (m, 25H), 1.40-1.10 (m, 5H).

### Synthesis Example of Compound (B-8)

The same operation was performed as in the synthesis of the compound (B-1), except that triphenylsulfonium bromide (the formula (2-1)) (14.9 g) was replaced with a compound represented by the following formula (2-3) (25.8 g) to synthesize. Thus, a compound (B-8) was obtained.

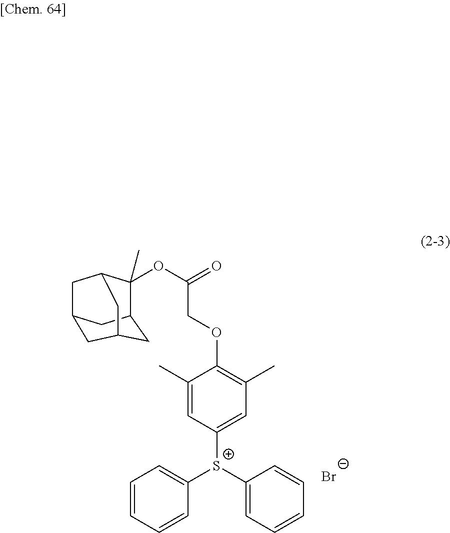 [SMILES: Cc1cc([S+](c2ccccc2)c2ccccc2)cc(C)c1OCC(=O)OC1(C)C2CC3CC(C2)CC1C3 ; [Br-]]

The compound (B-8) was analyzed by NMR.

¹H NMR (400 MHz, DMSO-d6) δ (ppm)=7.93-7.61 (m, 12H), 4.97 (m, 1H), 4.61 (s, 2H), 4.30-4.05 (m, 2H), 2.49-1.10 (m, 50H).

### Synthesis Example of Compound (B-9)

The same operation was performed as in the synthesis of the compound (B-1), except that the compound represented by the formula (1-1) (30 g) was replaced with a compound represented by the following formula (1-4) (27.8 g) to synthesize. Thus, a compound (B-9) was obtained.

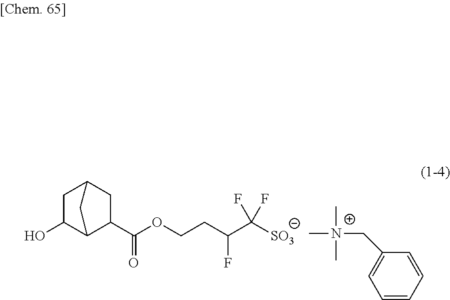

The compound (B-9) was analyzed by NMR.

¹H NMR (400 MHz, DMSO-d6) δ (ppm)=7.93-7.69 (m, 15H), 4.97 (m, 1H), 4.30-4.05 (m, 2H), 3.65-1.10 (m, 23H).

From the above results, it was confirmed that the compounds (B-1) to (B-9) had the following structures.

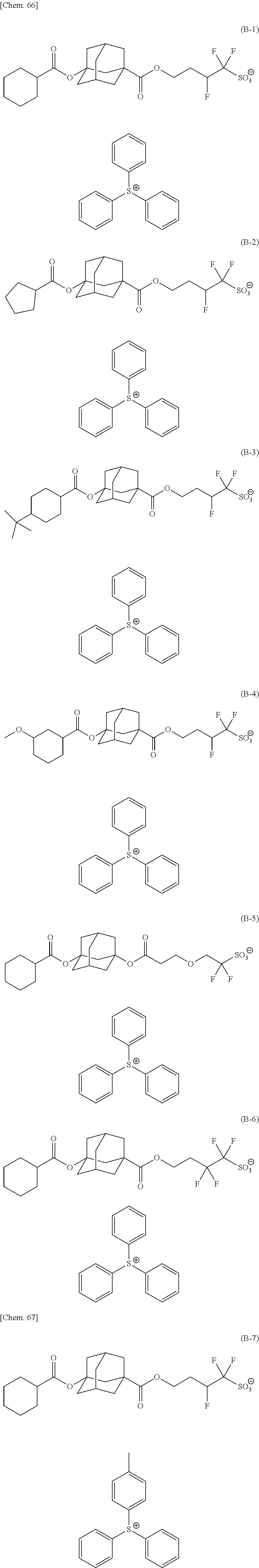

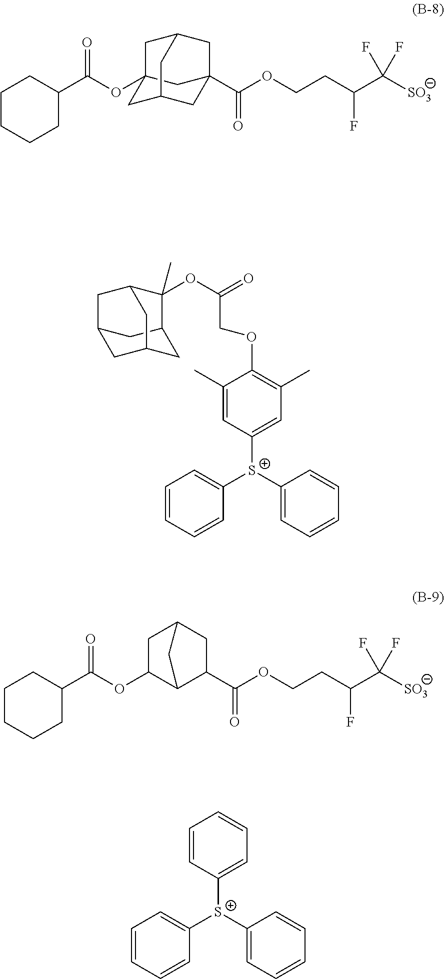

A polymer compound (A-1) containing structural units shown below in a composition ratio (molar ratio) of (1:m=50:50) was synthesized.

A weight average molecular weight (Mw) of the obtained polymer compound, calculated based on standard polystyrene, as determined by GPC measurement, was 11,000, and a molecular weight dispersity (Mw/Mn) thereof was 1.6.

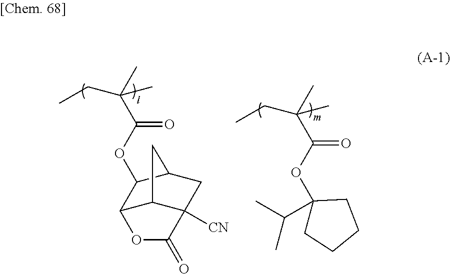

### <Preparation of Resist Composition>

### Examples 1 to 9 and Comparative Examples 1 to 5

Components shown in Table 1 were mixed and dissolved in the following solvent: S-1 to prepare resist compositions of each example.

S-1: A mixed solvent of 1750 parts by mass of propylene glycol monomethyl ether acetate, 640 parts by mass of propylene glycol monomethyl ether, and 800 parts by mass of cyclohexanone

TABLE 1

| | | | Component (B) | | Component (D) | | | | | | Stability |
|---|---|---|---|---|---|---|---|---|---|---|---|
| | Component (A) | | Acid | | Acid-diffusion | | Component (F) | | EL | | over |
| | | Part by | generating | Part by | controlling | Part by | Hydrophobic | Part by | evaluation | Sensitivity | time |
| | Resin | mass | agent | mass | agent | mass | resin | mass | [%] | [mJ/cm²] | [nm] |
| Example 1 | A-1 | 100 | B-1 | 9.32 | D-1 | 3.3 | F-1 | 4.4 | 12.7 | 22.4 | −0.36 |
| Example 2 | A-1 | 100 | B-2 | 9.15 | D-1 | 3.3 | F-1 | 4.4 | 12.2 | 21.9 | −0.42 |
| Example 3 | A-1 | 100 | B-3 | 10.01 | D-1 | 3.3 | F-1 | 4.4 | 13.1 | 23 | −0.38 |
| Example 4 | A-1 | 100 | B-4 | 9.69 | D-1 | 3.3 | F-1 | 4.4 | 12.8 | 22.5 | −0.4 |
| Example 5 | A-1 | 100 | B-5 | 9.47 | D-1 | 3.3 | F-1 | 4.4 | 12.3 | 23.1 | −0.45 |
| Example 6 | A-1 | 100 | B-6 | 9.54 | D-1 | 3.3 | F-1 | 4.4 | 12.5 | 21.9 | −0.41 |
| Example 7 | A-1 | 100 | B-7 | 9.49 | D-1 | 3.3 | F-1 | 4.4 | 12.6 | 23.5 | −0.39 |
| Example 8 | A-1 | 100 | B-8 | 12.39 | D-1 | 3.3 | F-1 | 4.4 | 12.7 | 23.9 | −0.42 |
| Example 9 | A-1 | 100 | B-9 | 8.83 | D-1 | 3.3 | F-1 | 4.4 | 11.8 | 21.8 | −0.4 |
| Comparative | A-1 | 100 | B-X1 | 9.66 | D-1 | 3.3 | F-1 | 4.4 | 12.6 | 22.1 | −1.21 |
| Example 1 | | | | | | | | | | | |
| Comparative | A-1 | 100 | B-X2 | 8.04 | D-1 | 3.3 | F-1 | 4.4 | 9.8 | 25.9 | −0.41 |
| Example 2 | | | | | | | | | | | |
| Comparative | A-1 | 100 | B-X3 | 9.59 | D-1 | 3.3 | F-1 | 4.4 | 14.1 | 26.5 | −0.4 |
| Example 3 | | | | | | | | | | | |
| Comparative | A-1 | 100 | B-X4 | 8.75 | D-1 | 3.3 | F-1 | 4.4 | 12.6 | 26.1 | −0.39 |
| Example 4 | | | | | | | | | | | |
| Comparative | A-1 | 100 | B-X5 | 9.56 | D-1 | 3.3 | F-1 | 4.4 | 14.2 | 26.9 | −0.38 |
| Example 5 | | | | | | | | | | | |

In Table 1, the abbreviations have the following meanings.

A-1: the above polymer compound A-1

B-1 to B-9: acid generating agents composed of the compounds represented by the above chemical formulae B-1 to B-9, respectively

B-X1 to B-X5: acid generating agents composed of the compounds represented by the following chemical formulas B-X1 to B-X5, respectively

D-1: an acid diffusion control agent composed of a compound represented by the following chemical formula D-1

F-1: a hydrophobic resin F-1 containing the following structural unit (composition ratio (molar ratio): 1/m=80/20, Mw: 25000, Mw/Mn: 1.5)

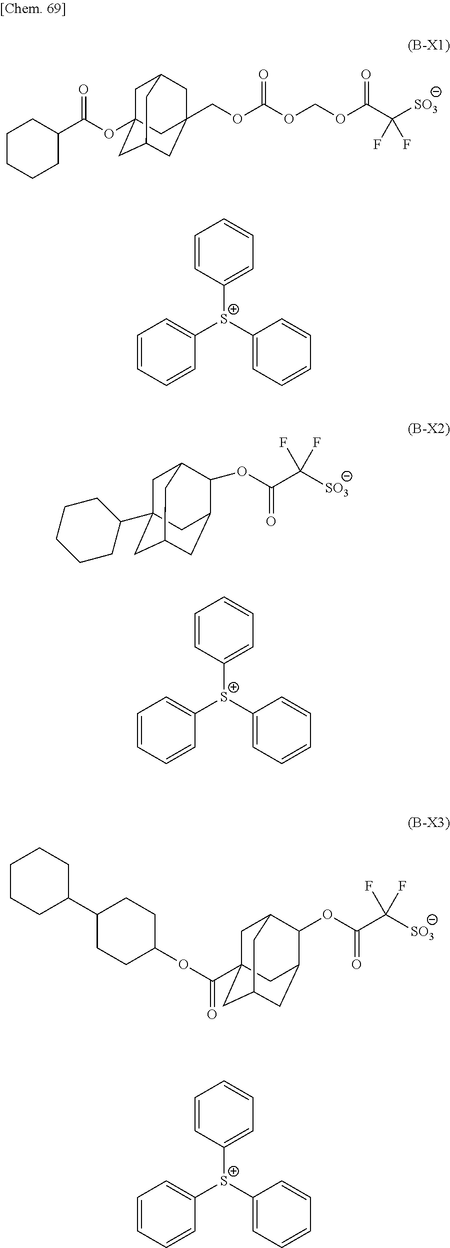

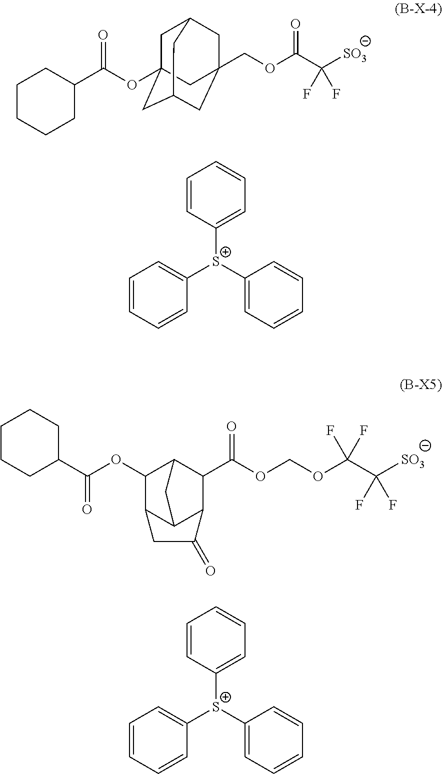

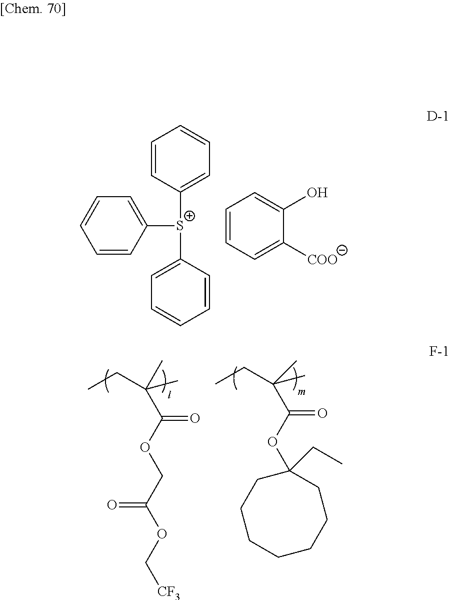

### <Formation of Resist Pattern>

An organic antireflection film composition “SOC110D” (manufactured by Brewer Science, Inc.) was applied onto a 12-inch silicon wafer using a spinner, and the composition was then baked and dried on a hot plate at 205° C. for 60 seconds to form an organic antireflection film having a film thickness of 137 nm.

Further, a hard mask “HM825” (manufactured by Brewer Science, Inc.) was applied onto the antireflection film using a spinner and dried by baking on a hot plate at 205° C. for 60 seconds to form a hard mask layer having a thickness of 30 nm.

The resist composition was applied onto the underlying substrate using a spinner, and was then prebaked (PAB) on a hot plate at 100° C. for 60 seconds and dried to form a resist film having a film thickness of 100 nm.

Using an ArF exposure apparatus XT1900Gi [manufactured by ASML; NA (numerical aperture)=1.35, Dipole 90X, Sigma (Ont 0.97, In 0.78) TE-pol, immersion medium: ultrapure water], an ArF excimer laser (193 nm) was selectively irradiated through a photomask (6% halftone). Thereafter, a PEB treatment was performed at 120° C. for 60 seconds.

Next, an alkaline development was performed at 23° C. for 15 seconds using a 2.38 mass % TMAH aqueous solution (product name: NMD-3, manufactured by Tokyo Ohka Kogyo Co., Ltd.), and then water rinsing was performed for 15 seconds using pure water, followed by shaking and drying. As a result, a line and space pattern (hereinafter, referred to as an LS pattern) having a line dimension of 40 nm and a pitch of 80 nm (a mask size of 40 nm) was formed.

### [Evaluation of Sensitivity (Optimal Exposure Amount)]

The line size was observed by a length measuring SEM (scanning electron microscope, acceleration voltage: 500 V, product name: CG5000, manufactured by Hitachi High-Technologies Corporation), and an exposure amount at which the LS pattern having a line dimension of 40 nm and a pitch of 80 nm was formed was shown in Table 1 as an optimal exposure amount (mJ/cm²).

### [Evaluation of Exposure Latitude (EL)]

When the exposure amount was varied around the above-described optimal exposure amount, a fluctuation range of the exposure amount that allows a line width of 40 nm+5% was determined, and this value was divided by the optimal exposure amount to be expressed as a percentage. The larger the value, the smaller the change in performance due to the change in exposure amount, and the better the exposure latitude.

### [Evaluation of Stability Over Time]

Each of the compositions in Examples and Comparative Examples was stored at room temperature for 2 months, and then exposed to light at the optimal exposure amount at the time of the initial evaluation. The line dimension was measured in the same manner as above, and the variation from the initial evaluation value was determined. The smaller the absolute value of this value, the less likely the resist composition is to deteriorate, and the better stability over time.

From the results shown in Table 1, it was confirmed that the resist compositions in Examples to which the present invention was applied were excellent in stability over time, and were capable of forming a resist pattern having both a high exposure latitude and a high sensitivity to a light source for exposure.

### INDUSTRIAL APPLICABILITY

According to the present invention, a resist composition capable of forming a resist pattern having excellent stability over time and having both a high exposure latitude and a high sensitivity to a light source for exposure, a method for forming a resist pattern using the resist composition, and a compound useful for the resist composition can be provided.

Although the present invention has been described in detail with reference to specific embodiments, it is apparent to those skilled in the art that various changes and modifications can be made without departing from the spirit and scope of the present invention.

The present application is based on a Japanese patent application (JP2022-119718) filed on Jul. 27, 2022, the disclosure of which is hereby incorporated by reference herein in its entirety.

What is claimed is:

1. A resist composition that generates an acid upon exposure and whose solubility with respect to a liquid developer changes by an action of the acid, the resist composition comprising:
a base material component (A) whose solubility with respect to a liquid developer changes by an action of an acid; and
an acid generating agent component (B) that generates an acid upon exposure, wherein
the acid generating agent component (B) comprises a compound represented by the following general formula (b1-1): 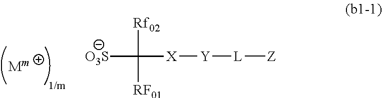
wherein Rf₀₁ and Rf₀₂ each independently represents a hydrogen atom, a fluorine atom, or a fluorinated alkyl group, and at least one of Rf₀₁ and Rf₀₂ comprises a fluorine atom, X represents a divalent linking group having a total of 4 or more atoms in a main chain, and the divalent linking group represents an alkylene group that may comprise an ester bond or an ether bond, a fluorinated alkylene group that may comprise an ester bond or an ether bond, or a combination thereof, any of ester bonds and ether bonds in X does not form an acetal bond adjacent to each other, Y represents a divalent polycyclic hydrocarbon group that may include a substituent, L represents a divalent linking group comprising an ester bond or an oxygen atom, a plurality of ester bonds and a plurality of oxygen atoms may be each bonded independently via an alkylene group, Z represents a substituent represented by the following formula (bz-1), m represents an integer of 1 or more, and M^{m+} represents an m-valent organic cation, 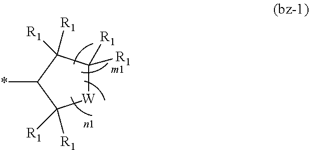
wherein each R₁ independently represents a hydrogen atom, a halogen atom, a linear or branched alkyl group in which a part of carbon atoms may be replaced by a heteroatom, a linear or branched acyl group in which a part of carbon atoms may be replaced by a heteroatom, or a linear or branched alkyloxycarbonyl group in which a part of carbon atoms may be replaced by a heteroatom, W represents an oxygen atom, a sulfur atom, or —NRⁿ—, Rⁿ represents a hydrogen atom, or a linear or branched alkyl group, m1 and n1 each represents an integer of 0 to 4, m1+n1 represents an integer of 0 to 4, and * represents a bond.

2. The resist composition according to claim 1, wherein the polycyclic hydrocarbon group represented by Y has an adamantane skeleton or a norbornane skeleton.

3. The resist composition according to claim 1, wherein M^{m+} in the general formula (b1-1) is a cation represented by the following general formula (b-2): 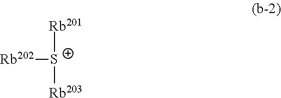
wherein Rb²⁰¹ represents an aryl group that may include a substituent, Rb²⁰² and Rb²⁰³ each independently represents an aryl group that may include a substituent, an alkyl group that may include a substituent, or an alkenyl group that may include a substituent, and Rb²⁰¹ to Rb²⁰³ may be bonded to each other to form a ring together with a sulfur atom in the general formula (b-2).

4. The resist composition according to claim 1, wherein a content of the acid generating agent component (B) is 1 part by mass to 40 parts by mass with respect to 100 parts by mass of the base material component (A).

5. The resist composition according to claim 1, wherein the base material component (A) comprises a polymer compound (A1) having a structural unit (a1) represented by the following general formula (a1-1): 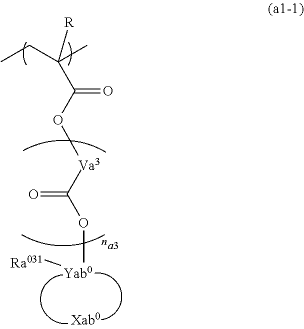
wherein R represents a hydrogen atom, an alkyl group having 1 to 5 carbon atoms, or a halogenated alkyl group having 1 to 5 carbon atoms, Va³ represents a divalent linking group, na₃ represents an integer of 0 to 2, Ra⁰³¹ represents an alkyl group, and Yab⁰ represents a carbon atom, Xab⁰ represents a group that forms an alicyclic hydrocarbon group together with Yab⁰, and a part or all of hydrogen atoms of the alicyclic hydrocarbon group may be replaced.

6. The resist composition according to claim 5, wherein the polymer compound (A1) further comprises a structural unit (a2) comprising a lactone-containing cyclic group, a cyclic group containing —SO₂—, or a carbonate-containing cyclic group.

7. The resist composition according to claim 1, further comprising:
an acid diffusion control agent (d) comprising a compound represented by any one of the following general formulae (d1-1) to (d1-3): 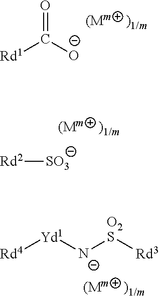
wherein Rd¹ to Rd⁴ each represents a cyclic group that may include a substituent, a chain alkyl group that may include a substituent, or a chain alkenyl group that may include a substituent, and in Rd², no fluorine atom is bonded to a carbon atom adjacent to an S atom, Yd¹ represents a single bond or a divalent linking group, m represents an integer of 1 or more, and each M^{m+} independently represents an m-valent organic cation.

8. A method for forming a resist pattern comprising:
forming a resist film on a support using the resist composition according to claim 1;
exposing the resist film; and
developing the resist film to form a resist pattern.

9. A salt represented by the following general formula (b1-1): 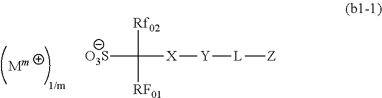
wherein Rf₀₁ and Rf₀₂ each independently represents a hydrogen atom, a fluorine atom, or a fluorinated alkyl group, and at least one of Rf₀₁ and Rf₀₂ comprises a fluorine atom, X represents a divalent linking group having a total of 4 or more atoms in a main chain, and the divalent linking group represents an alkylene group that may comprise an ester bond or an ether bond, a fluorinated alkylene group that may comprise an ester bond or an ether bond, or a combination thereof, any of ester bonds and ether bonds in X does not form an acetal bond adjacent to each other, Y represents a divalent polycyclic hydrocarbon group that may include a substituent, L represents a divalent linking group comprising an ester bond or an oxygen atom, a plurality of ester bonds and a plurality of oxygen atoms may be each bonded independently via an alkylene group, Z represents a substituent represented by the following formula (bz-1), m represents an integer of 1 or more, and M^{m+} represents an m-valent organic cation, 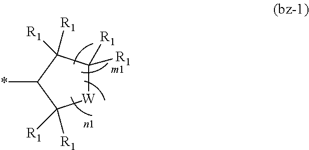
wherein each R¹ independently represents a hydrogen atom, a halogen atom, a linear or branched alkyl group in which a part of carbon atoms may be replaced by a heteroatom, a linear or branched acyl group in which a part of carbon atoms may be replaced by a heteroatom, or a linear or branched alkyloxycarbonyl group in which a part of carbon atoms may be replaced by a heteroatom, W represents an oxygen atom, a sulfur atom, or an amino group that may be replaced by a linear or branched alkyl group, m1 and n1 each represent an integer of 0 to 4, m1+n1 is an integer of 0 to 4, and * represents a bond.

10. The salt according to claim 9, wherein the polycyclic hydrocarbon group represented by Y in the general formula (b1-1) has an adamantane skeleton or a norbornane skeleton.

11. The salt according to claim 9, wherein M^{m+} in the general formula (b1-1) is a cation represented by the following general formula (b-2): 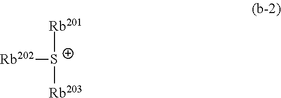
wherein Rb²⁰¹ represents an aryl group that may include a substituent, Rb²⁰² and Rb²⁰³ each independently represents an aryl group that may include a substituent, an alkyl group that may include a substituent, or an alkenyl group that may include a substituent, and Rb²⁰¹ to Rb²⁰³ may be bonded to each other to form a ring together with a sulfur atom in the general formula (b-2).
